(19) United States
(12) Patent Application Publication
(10) Pub. No.: US 2026/0250312 A1
(43) Pub. Date: Aug. 27, 2026

(54) ANTIBACTERIAL COMPOUNDS ELIMINATING DORMANT BACTERIAL CELLS

(71) Applicants: UNIVERSITÉ D'AIX-MARSEILLE, Marseille (FR); INSTITUT NATIONAL DE LA SANTÉ ET DE LA RECHERCHE MÉDICALE (INSERM), Paris (FR); OLGRAM, Bréhan (FR)

(72) Inventors: Jean-Michel BRUNEL, Marseille (FR); Pierre ROCHETEAU, Genève (CH)

(73) Assignees: UNIVERSITÉ D'AIX-MARSEILLE, Marseille (FR); INSTITUT NATIONAL DE LA SANTÉ ET DE LA RECHERCHE MÉDICALE (INSERM), Paris (FR); OLGRAM, Bréhan (FR)

(21) Appl. No.: 18/858,810

(22) PCT Filed: Apr. 26, 2023
(86) PCT No.: PCT/EP2023/060895

§ 371 Date: Oct. 22, 2024

(30) Foreign Application Priority Data
Apr. 26, 2022 (EP) 22305619.3

(51) Int. Cl. C07J 9/00; A61K 31/575; A61P 31/04

(52) U.S. Cl. CPC C07J 9/00; A61K 31/575; A61P 31/04

(57) ABSTRACT

Compounds of formula (I):

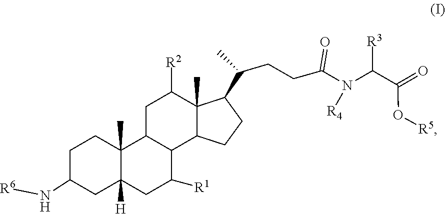

or a pharmaceutically acceptable salt and/or solvates thereof. Also, the use of the compounds of formula (I), or a pharmaceutically acceptable salt and/or solvates thereof, as anti-infective agents. In particular, these compounds may be used as antibacterial and/or anti-persisters agents, especially for the treatment of infectious diseases.

### FIELD OF INVENTION

The present invention relates to aminosteroid derivatives of formula (I) for use as anti-infective agents, in particular for use as antibacterial and/or anti-persisters agents. The compounds of the invention are especially useful in the treatment of infectious diseases.

### BACKGROUND OF INVENTION

Bacteria cause many infections that are harmful and sometime lethal to human and animals. For many reasons including overuse of antibiotics, more and more bacterial strains exhibit multi-drug resistance, so that the efficiency of the treatment of bacterial infectious diseases is significantly limited. Drug resistance is however not the sole responsible of the loss of efficacy of antibacterials. Indeed, even high amounts of antimicrobials may not be able to eliminate the “dormant” cells, in particular persisters and Viable But Non Culturable cells (VBNCs). Surviving dormant cells play a critical role in the infection relapse. In addition of limiting the effect of antibacterials, dormant cells may be responsible of chronic diseases. By contrast with drug resistance, persistence and activation of dormant cells is strongly associated with individual and environmental factors.

Very few options are currently available to eliminate dormant cells, because most treatments have been designed to kill active cells. One strategy to specifically target persisters is the use of drugs, e.g., antimicrobial peptides, that interact directly with the cell membrane, so that they are effective even on a metabolically inactive bacterium. Another option is to first reactivate the dormant cells, for example with saccharides, then use common antibacterials to eliminate the reactivated cells. The methods are however of limited applicability; and so far, no convenient therapeutic solution for persisters removal is available.

Squalamine is a natural compound that was isolated in 1993 from the tissues of a small shark Squalus acanthias, of the following formula.

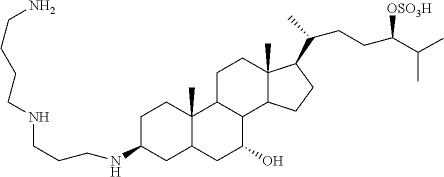

It is an active substance exhibiting in particular antiangiogenic activity against cells as well as strong antiviral and antibacterial activity. Squalamine was also evidenced as being efficient against antimicrobial-resistant bacteria such as Gram-negative and Gram-positive bacteria. Chemically, squalamine is a polycationic aminosterol: it comprises a nonpolar central moiety (cholestane-type) and two polar ends, namely a polyamine chain and a sulphate group. It has thus an amphiphilic character and is also water-soluble. Squalamine was initially considered of interest for its antiangiogenic and antimicrobial properties on a variety of Gram-positive bacteria (Staphylococcus aureus, Enterococcus faecalis) and Gram-negative bacteria (Escherichia coli, Pseudomonas aeruginosa), fungi (Candia albicans, Candida tropicalis) and protozoa.

As natural sourcing of squalamine is very limited, derivatives or analogues of squalamine have been synthetized and investigated. In particular, aminosteroidal analogs comprising a polyamine chain on 10,13-dimethyl-17-octane-cholestane or cholestene rings in position 3 or 7 have been described as shown on formula a, b, c and d below (WO2011/067501 A1, Brunel, J.-M. et al.).

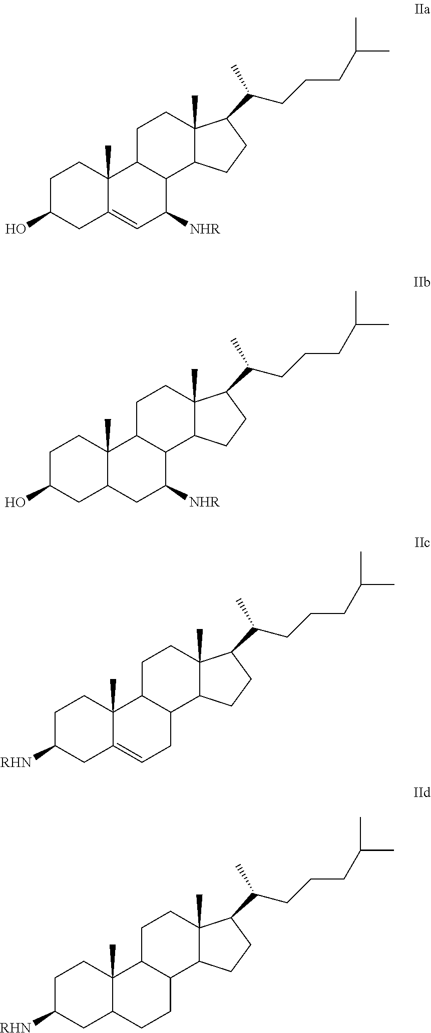

These compounds have squalamine-like antibacterial activity against various drug-resistant Gram-positive and Gram-negative bacteria (WO 2011/067501, Brunel, J.-M. et al.). These derivatives have been suggested in particular for use in a curative treatment of pulmonary infections through aerosol route. However, it was observed that some of these compounds exhibit significant cytotoxicity. Moreover, the compounds of formula IIc and IId above have a weak activity against some Gram-negative bacteria such as Escherichia coli. Moreover, no anti-persisters effect was reported for these molecules.

Thus, there is still a need for new derivatives or analogues of squalamine presenting improved biological and/or chemical properties, preferably analogues of squalamine with anti-persisters activity.

The Applicant have identified that new squalamine analogs, namely the aminosteroid derivatives of formula (I), exhibit good antibacterial activity against a variety of Gram-positive and Gram-negative bacteria and/or have a low cytotoxicity. In particular, the compounds of the invention may be used as anti-persisters agents. The compounds of the inventions are also simpler to synthetize compared to squalamine.

### SUMMARY

This invention relates to a compound of Formula (I)

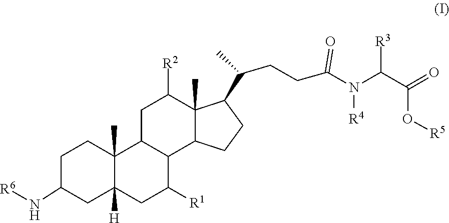

or a pharmaceutically acceptable salt and/or solvate thereof; wherein R¹, R², R³, R⁴, R₅ and R⁶ are as defined herein.

According to one embodiment, the compound according to the invention is selected from
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)valinate
methyl((4R)-4-((3S,5S,7R,10S,13R,17R)-3-((3-(4-(3-aminopropyl)piperazin-1-yl)propyl)amino)-7-hydroxy-10,13-dimethylhexadecahydro-1H-cyclopenta[a]phenanthren-17-yl)pentanoyl)valinate
methyl((4R)-4-((3S,5S,7R,10S,13R,17R)-3-((3-(bis(3-aminopropyl)amino)propyl)amino)-7-hydroxy-10,13-dimethylhexadecahydro-1H-cyclopenta[a]phenanthren-17-yl)pentanoyl)valinate
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)glycinate
methyl((4R)-4-((3S,5S,7R, 10S,13R,17R)-3-((3-(bis(3-aminopropyl)amino)propyl)amino)-7-hydroxy-10,13-dimethylhexadecahydro-1H-cyclopenta[a]phenanthren-17-yl)pentanoyl)glycinate
methyl((4R)-4-((3S,5S,7R,10S,13R,17R)-3-((3-(4-(3-aminopropyl)piperazin-1-yl)propyl)amino)-7-hydroxy-10,13-dimethylhexadecahydro-1H-cyclopenta[a]phenanthren-17-yl)pentanoyl)glycinate
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((2-aminoethyl)amino)-7-hydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((10-aminodecyl)amino)-7-hydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((2-((2-((2-aminoethyl)amino)ethyl)amino)ethyl)amino)-7-hydroxy-10,13-dimethylhexadecahydro-1H-cyclopenta[a]phenanthren-17-yl)pentanoyl)-D-phenylalaninate
methyl((4R)-4-((3S,5S,7R,10S,13R,17R)-3-((3-((3-((3-aminopropyl)amino)propyl)amino)propyl)amino)-7-hydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((2-((2-((2-aminoethyl)amino)ethyl)amino)ethyl)amino)-7-hydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-valinate
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-phenylalaninate
methyl((4R)-4-((3S,5S,7R,10S,13R,17R)-3-((2-aminoethyl)amino)-7-hydroxy-10,13-dimethylhexadecahydro-1H-cyclopenta[a]phenanthren-17-yl)pentanoyl)-L-phenylalaninate methyl((4R)-4-((3S,5S,7R,10S,13R,17R)-3-((3-aminopropyl)amino)-7-hydroxy-10,13-dimethylhexadecahydro-1H-cyclopenta[a]phenanthren-17-yl)pentanoyl)-L-phenylalaninate
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((3-aminopropyl)amino)-7-hydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((2-((2-aminoethyl)amino)ethyl)amino)-7-hydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((3-((3-((3-aminopropyl)amino)propyl)amino)propyl)amino)-7-hydroxy-10,13-dimethylhexadecahydro-1H-cyclopenta[a]phenanthren-17-yl)pentanoyl)-D-phenylalaninate
methyl((4R)-4-((3S,5S,7R,10S,13R,17R)-3-((6-aminohexyl)amino)-7-hydroxy-10,13-dimethylhexadecahydro-1H-cyclopenta[a]phenanthren-17-yl)pentanoyl)-D-valinate methyl((4R)-4-((3S,5S,7R,10S,13R,17R)-3-((2-((2-aminoethyl)amino)ethyl)amino)-7-hydroxy-10,13-dimethylhexadecahydro-1H-cyclopenta[a]phenanthren-17-yl)pentanoyl)-L-phenylalaninate
methyl((4R)-4-((3S,5S,7R,10S,13R,17R)-3-((3-(4-(3-aminopropyl)piperazin-1-yl)propyl)amino)-7-hydroxy-10,13-dimethylhexadecahydro-1H-cyclopenta[a]phenanthren-17-yl)pentanoyl)-L-phenylalaninate methyl((4R)-4-((3S,5S,7R,10S,13R,17R)-3-((3-((3-aminopropyl)(methyl)amino)propyl)amino)-7-hydroxy-10,13-dimethylhexadecahydro-1H-cyclopenta[a]phenanthren-17-yl)pentanoyl)-L-valinate
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-phenylalaninate
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-phenylalaninate
methyl((4R)-4-((3S,5S,7R, 10S,13R,17R)-3-((4-aminobutyl)amino)-7-hydroxy-10,13-dimethylhexadecahydro-1H-cyclopenta[a]phenanthren-17-yl)pentanoyl)-L-phenylalaninate
methyl((4R)-4-((3S,5S,7R,10S,13R,17R)-3-((3-((3-aminopropyl)(methyl)amino)propyl)amino)-7-hydroxy-10,13-dimethylhexadecahydro-1H-cyclopenta[a]phenanthren-17-yl)pentanoyl)-L-phenylalaninate methyl((4R)-4-((3S,5S,7R,10S,13R,17R)-3-((10-aminodecyl)amino)-7-hydroxy-10,13-dimethylhexadecahydro-1H-cyclopenta[a]phenanthren-17-yl)pentanoyl)-L-phenylalaninate
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-alaninate
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-leucinate
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-leucinate
methyl((4R)-4-((3S,5S,7R,10S,13R,17R)-3-((3-(bis(3-aminopropyl)amino)propyl)amino)-7-hydroxy-10,13-dimethylhexadecahydro-1H-cyclopenta[a]phenanthren-17-yl)pentanoyl)-L-phenylalaninate
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-alaninate
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-tyrosinate
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-L-tyrosinate
methyl (2S,3S)-2-((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanamido)-3-methylpentanoate
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-tyrosinate
methyl((4R)-4-((3S,5S,7R,10S,13R,17R)-3-((4-aminobutyl)amino)-7-hydroxy-10,13-dimethylhexadecahydro-1H-cyclopenta[a]phenanthren-17-yl)pentanoyl)-L-valinate
methyl (2S,3R)-2-((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanamido)-3-methylpentanoate
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-prolinate
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-D-prolinate
methyl((4R)-4-((3S,5S,7R, 10S,13R,17R)-3-((2-((2-((2-((2-aminoethyl)amino)ethyl)amino)ethyl)amino)ethyl)amino)-7-hydroxy-10,13-dimethylhexadecahydro-1H-cyclopenta[a]phenanthren-17-yl)pentanoyl)-L-valinate
methyl (2S,3R)-2-((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanamido)-3-methylpentanoate
methyl((4R)-4-((3S,5S,7R,10S,12S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)pentanoyl)glycinate
methyl((4R)-4-((3S,5R,10S,12S,13R,17R)-3-((3-((4-((3-aminopropyl)amino)butyl)amino)propyl)amino)-12-hydroxy-10,13-dimethylhexadecahydro-1H-cyclopenta[a]phenanthren-17-yl)pentanoyl)-D-valinate
methyl((4R)-4-((3S,5R,10S,12S,13R,17R)-3-((3-(4-(3-aminopropyl)piperazin-1-yl)propyl)amino)-12-hydroxy-10,13-dimethylhexadecahydro-1H-cyclopenta[a]phenanthren-17-yl)pentanoyl)-D-valinate
methyl((4R)-4-((3S,5R, 10S,12S,13R,17R)-3-((3-(bis(3-aminopropyl)amino)propyl)amino)-12-hydroxy-10,13-dimethylhexadecahydro-1H-cyclopenta[a]phenanthren-17-yl)pentanoyl)-D-valinate
methyl((4R)-4-((3S,5R,10S,12S,13R,17R)-3-((3-(bis(3-aminopropyl)amino)propyl)amino)-12-hydroxy-10,13-dimethylhexadecahydro-1H-cyclopenta[a]phenanthren-17-yl)pentanoyl)-L-phenylalaninate
methyl((4R)-4-((3S,5R,10S,13R,17R)-3-((3-(4-(3-aminopropyl)piperazin-1-yl)propyl)amino)-10,13-dimethylhexadecahydro-1H-cyclopenta[a]phenanthren-17-yl)pentanoyl)-L-valinate
methyl((4R)-4-((3S,5R,10S,13R,17R)-3-((3-((4-((3-aminopropyl)amino)butyl)amino)propyl)amino)-10,13-dimethylhexadecahydro-1H-cyclopenta[a]phenanthren-17-yl)pentanoyl)-L-valinate
methyl((4R)-4-((3S,5S,7R,10S,12S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)pentanoyl)-D-valinate
methyl((4R)-4-((3S,5S,7R,10S,12S,13R,17R)-3-((3-(4-(3-aminopropyl)piperazin-1-yl)propyl)amino)-7,12-dihydroxy-10,13-dimethylhexadecahydro-1H-cyclopenta[a]phenanthren-17-yl)pentanoyl)-D-valinate
methyl((4R)-4-((3S,5S,7R,10S,12S,13R,17R)-3-((3-(bis(3-aminopropyl)amino)propyl)amino)-7,12-dihydroxy-10,13-dimethylhexadecahydro-1H-cyclopenta[a]phenanthren-17-yl)pentanoyl)-D-valinate
methyl((4R)-4-((3S,5S,7R,10S,12S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)pentanoyl)-D-tyrosinate
   and pharmaceutically acceptable salts and/or solvates thereof.

This invention further relates to a pharmaceutical composition comprising a compound according to the invention and at least one pharmaceutically acceptable carrier.

This invention further relates to a compound or pharmaceutical composition according to the invention for use as a medicament. This invention further relates to a compound or pharmaceutical composition according to the invention for use in the treatment of an infectious disease; preferably in the treatment of a bacterial disease, a viral disease, a fungal disease or a parasitic disease. According to one embodiment, the infectious disease is a bacterial or fungal disease selected from cystic fibrosis, urinary tract infection and chronic otitis; and/or caused by Gram-positive bacteria selected from Staphylococcus bacteria, Enterococcus bacteria and Mycobacterium bacteria.

This invention further relates to a compound selected from
methyl((4R)-4-((3S,5S,7R, 10S,13R,17R)-3-((2-((2-((2-aminoethyl)amino)ethyl)amino)ethyl)amino)-7-hydroxy-10,13-dimethylhexadecahydro-1H-cyclopenta[a]phenanthren-17-yl)pentanoyl)-D-phenylalaninate
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-tyrosinate
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-D-prolinate
methyl((4R)-4-((3S,5S,7R,10S,12S,13R,17R)-3-((3-(bis(3-aminopropyl)amino)propyl)amino)-7,12-dihydroxy-10,13-dimethylhexadecahydro-1H-cyclopenta[a]phenanthren-17-yl)pentanoyl)-D-valinate
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-valinate
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-phenylalaninate
methyl (2S,3R)-2-((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanamido)-3-methylpentanoate
methyl((4R)-4-((3S,5R,10S,12S,13R,17R)-3-((3-((4-((3-aminopropyl)amino)butyl)amino)propyl)amino)-12-hydroxy-10,13-dimethylhexadecahydro-1H-cyclopenta[a]phenanthren-17-yl)pentanoyl)-D-valinate
   and pharmaceutically acceptable salts and/or solvates thereof;

for use as anti-persisters agent in the treatment of an infectious disease; preferably in the treatment of a bacterial disease or a fungal disease.

This invention further relates to the non-therapeutic use of a compound according to the invention as anti-infective agent for the disinfection of a surface and/or the purification of a liquid, preferably as anti-persisters agent for the disinfection of a surface and/or the purification of a liquid; wherein the surface or the liquid is not part of a human or animal body.

This invention further relates to a process for manufacturing a compound according to the invention, wherein the process comprises: (a) a step of reacting the carboxylic acid function in position 20 of a bile acid with the secondary amine function of an amino acid, thereby obtaining an amide; (b) a step of oxidizing the hydroxyl (OH) in position 3 of the bile acid intermediate obtained in step (a), thereby obtaining a ketone; (c) a step of reacting the ketone of the bile acid intermediate obtained in step (b) with a primary amine of formula R⁶NH₂, thereby obtaining an imine; and (d) a step of reduction of the imine obtained in step (c), thereby obtaining the compound of formula (I) or the pharmaceutically acceptable salt and/or solvate thereof.

### Definitions

In the present invention, the following terms have the following meanings.

### Chemical Definitions

Where chemical substituents are combinations of chemical groups, the point of attachment of the substituent to the molecule is by the last chemical group recited on the right of the name of the substituent. For example, an arylalkyl substituent is linked to the rest of the molecule through the alkyl moiety and it may by represented as follows: “aryl-alkyl-”. Unless otherwise indicated, the compounds were named using ChemDraw® Professional 15.0 (PerkinElmer).
“Alkoxy” refers to an alkyl-O- group.
“Alkyl” refers to a saturated linear or branched hydrocarbon chain, typically comprising from 1 to 12 carbon atoms, preferably from 1 to 8 carbon atoms, more preferably from 1 to 4 carbon atoms. In the present invention, alkyl groups may be monovalent or polyvalent (i.e., “alkylene” groups as defined herein are encompassed in “alkyl” definition) but alkyl groups are typically monovalent. Non-limiting examples of alkyl groups include methyl, ethyl, n-propyl, i-propyl, n-butyl, i-butyl, s-butyl and t-butyl, pentyl and its isomers (e.g., n-pentyl, iso-pentyl), and hexyl and its isomers (e.g., n-hexyl, iso-hexyl). Preferred alkyl groups include methyl, ethyl, n-propyl, i-propyl, n-butyl, s-butyl and t-butyl.
“Alkylene” refers to a divalent alkyl group. Non-limiting examples of alkylene groups include methylene, ethylene, n-propylene, i-propylene, divalent butyl, divalent pentyl and divalent hexyl. Preferred alkylene groups include methylene, ethylene, n-propylene, n-butylene and n-butylene.
“Amine” refers to derivatives of ammonia (NH₃), wherein one or more hydrogen atoms have been replaced by a substituent such as, for example, alkyl or aryl.
“Aryl” refers to a cyclic, polyunsaturated, aromatic hydrocarbyl group comprising at least one aromatic ring and comprising from 5 to 12 carbon atoms, preferably from 6 to 10 carbon atoms. Aryl groups may have a single ring (e.g., phenyl) or multiple aromatic rings fused together (e.g., naphthyl) or linked covalently. The aromatic ring may optionally include one to two additional rings (either cycloalkyl, heterocycloalkyl or heteroaryl) fused thereto. This definition of “aryl” encompasses the partially hydrogenated derivatives of the carbocyclic systems enumerated herein, as long as at least one ring is aromatic. Non-limiting examples of aryl groups include phenyl, biphenyl, biphenylenyl, 5- or 6-tetralinyl, naphthalen-1- or -2-yl, 4-, 5-, 6 or 7-indenyl, 1-2-, 3-, 4- or 5-acenaphthylenyl, 3-, 4- or 5-acenaphthenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3—, 4- or 5-pyrenyl. A particularly preferred aryl group is phenyl.
“Cycloalkyl” refers to a cyclic monovalent alkyl group as defined herein comprising from 3 to 11 carbon atoms, preferably from 4 to 9 carbon atoms, more preferably from 5 to 7 carbon atoms. This definition of “cycloalkyl” encompasses polycyclic cycloalkyls (e.g., bicycles) and bridged cycloalkyl structures.
“Cₓ-C_{y}” or “(Cₓ-C_{y})” preceding the name of a group means that the group comprises from x to y carbon atoms, in accordance to common terminology in the chemistry field.
“Heteroaryl” refers to aromatic rings or aromatic ring systems comprising from 5 to 12 carbon atoms, preferably from 6 to 10 carbon atoms, having one or two rings which are fused together or linked covalently, wherein at least one ring is aromatic, and wherein one or more carbon atoms in one or more of these rings is replaced by oxygen, nitrogen and/or sulfur atoms. The nitrogen and sulfur heteroatoms may optionally be oxidized and the nitrogen heteroatoms may optionally be quaternized. Non-limiting examples of heteroaryl groups include furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl and quinoxalinyl.
“Heteroalkyl” refers to an alkyl group as defined herein wherein one or more carbon atoms are replaced by a heteroatom selected from oxygen, nitrogen and sulfur. In heteroalkyl groups, the heteroatoms are bound along the alkyl chain only to carbon atoms, i.e., each heteroatom is separated from any other heteroatom by at least one carbon atom. The nitrogen and sulfur heteroatoms may optionally be oxidized and the nitrogen heteroatoms may optionally be quaternized. Heteroalkyl groups may further include one or more ═O and/or ═S groups. A heteroalkyl is bound to another group or molecule only through a carbon atom, i.e., the binding atom is not selected among the heteroatoms included therein. Non-limiting examples of heteroalkyl include alkoxy, ethers and polyethers, secondary and tertiary amines and polyamines, thioethers and polythioethers, and combinations thereof.
“Heterocycloalkyl” refers to a cyclic monovalent heteroalkyl, typically comprising from 2 to 7 carbon atoms, preferably from 3 to 6 carbon atoms, more preferably from 4 to 5 carbon atoms. Heterocycloalkyl are typically 3- to 7-membered, preferably 5- or 6-membered. Heterocycloalkyl are typically monocyclic or bicyclic, preferably monocyclic. This definition encompasses polycyclic heterocycloalkyls (e.g., bicycles) and bridged heterocycloalkyl structures. Non-limiting examples of heterocycloalkyl include monovalent or divalent aziridine, pyrrolidine, piperidine, piperazine, morpholine, thiomorpholine, tetrahydrofuran and tetrahydropyran.
“Heterocyclyl” collectively refers to “heterocycloalkyl” and “heteroaryl” groups as defined herein.
“Hydroxy” refers to the —OH group.

### General Definitions

“About” is used herein to mean approximately, roughly, around, or in the region of. The term “about” preceding a figure means more or less 10% of the value of the figure. When the term “about” is used in conjunction with a numerical range, it modifies that range by extending the boundaries above and below the numerical values set forth by 10%.
“Administration”, or a variant thereof (e.g., “administering”), means providing a therapeutic agent (e.g., a compound of the invention) alone or as part of a pharmaceutically acceptable composition, to the patient in whom/which the condition, symptom, or disease is to be treated.
“Comprise” or a variant thereof (e.g., “comprises”, “comprising”) is used herein according to common patent application drafting terminology. Hence, “comprise” preceded by an object and followed by a constituent means that the presence of a constituent in the object is required (typically as a component of a composition), but without excluding the presence of any further constituent(s) in the object. Moreover, any occurrence of “comprise” or a variant thereof herein also encompasses narrower expression “substantially consist of”, further narrower expression “consist of” and any variants thereof (e.g., “consists of”, “consisting of”), unless otherwise stated.
“Infection” refers to any undesired presence and/or growth of pathogen (typically bacteria, viruses, fungi or parasites) in a subject. Such undesired presence of microorganism may have a negative effect on the host subject's health and well-being. While the term “infection” should not be taken as encompassing the normal growth and/or presence of microorganism which are normally present in the subject, for example in the digestive tract of the subject, it may encompass the pathological overgrowth of such microorganism. Infections may be caused by the growth and/or presence of microorganism, such as bacteria, viruses, fungi or parasites. “Chronic infection”, “relapsing infection”, “recalcitrant infection” and “persistent infection” refer to bacterial infection which resists to the host immune system and antibiotic treatments and is capable of reactivation into clinically significant disease with chronic symptoms.
“Infectious disease” refers to a pathologic condition or disorder resulting from an infection. Examples of specific infections include “bacterial disease”, “viral disease”, “fungal disease” and “parasitic disease”, which are infectious diseases caused respectively by bacteria, viruses, fungi or parasites. Therapeutic agents for the treatment of infectious diseases are “anti-infective” agents.
“Human” refers to a male or female human subject at any stage of development, including neonate, infant, juvenile, adolescent and adult.
“Kit” or “Kit of parts” are synonyms and refer to any manufacture (e.g., a package or a container) comprising a pharmaceutical composition comprising the compound according to the present invention. The kit may be promoted, distributed, or sold as a unit for performing the methods of the present invention.
“Persisters” refers to any type of dormant variants of regular cells, in particular persisters and viable but non-culturable cells (VBNCs). Persisters are susceptible to cause an infectious disease. Persisters are typically bacterial, however fungal persister cells, and yeast persister cells are also encompassed in this definition. Persisters represent a small subpopulation of genetically identical, metabolically slow-growing cells which spontaneously enter a dormant, nondividing state and can survive to extremely high antibiotic doses. When a population is treated with an antibiotic, regular cells die, whereas persisters survive. In order to kill, antibiotics require active targets, which explains persisters tolerance. By contrast, resistance mechanisms prevent antibiotics from binding to their targets. Resistance is measured by observing the ability of cells to grow in the presence of antibiotic. For the most part, the molecular mechanisms leading to persistence are unknown. As used herein, “persister cell” refers to metabolic variants of wild type microbial cells that are phenotypically characterized by their slow growth rate, which is typically 30%, 25%, 20%, 15%, 10%, 5% or less of the growth rate of the wild-type counterpart. In some embodiments, the persister cells are dormant and have, for example, no detectable cell division in a 24-hour period. Further, persister cells typically form colonies that are approximately 30%, 25%, 20%, 15%, 10%, 5% or less of the size of the colonies formed by their wild-type counterparts.
“Persister-related infection” refers to any infection in which persister cells are implicated.
“Patient” refers to a subject who/which is awaiting the receipt of, or is receiving medical care or was/is/will be the object of a medical procedure, or is monitored for the development of the targeted disease or condition, such as, for example, an infectious disease.
“Pharmaceutically acceptable” means that the ingredients of a composition are compatible with each other and not deleterious to the patient to which/whom it is administered.
“Pharmaceutically acceptable carrier” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, e.g., FDA Office or EMA. Examples of pharmaceutically acceptable carriers include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene- polyoxypropylene- block polymers, polyethylene glycol and wool fat.
“Prodrug” refers to a pharmacologically acceptable derivative of a therapeutic agent (e.g., a compound of the invention) whose in vivo biotransformation product is the therapeutic agent (active drug). Prodrugs are typically characterized by increased bioavailability and are readily metabolized in vivo into the active compounds. Non-limiting examples of prodrugs include amide prodrugs and carboxylic acid ester prodrugs, in particular alkyl esters, cycloalkyl esters and aryl esters.
“Selected from” is used herein according to common patent application drafting terminology, to introduce a list of elements among which one or more item(s) is (are) selected. Any occurrence of “selected from” in the specification may be replaced by “selected from the group comprising or consisting of” and reciprocally without changing the meaning thereof.
“Solvate” refers to molecular complex comprising a compound along with stoichiometric or sub-stoichiometric amounts of one or more molecules of one or more solvents, typically the solvent is a pharmaceutically acceptable solvent such as, for example, ethanol. The term “hydrate” refers to a solvate when the solvent is water (H₂O).
“Subject” refers to an animal, typically a warm-blooded animal, preferably a mammal. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human. In one embodiment, the subject is a “patient” as defined herein. In one embodiment, the subject is an adult (for example a subject above the age of 18). In one embodiment, the subject is a child (for example a subject below the age of 18). In one embodiment, the subject is a male. In one embodiment, the subject is a female. In one embodiment, the subject is affected, preferably is diagnosed, with an infectious disease. In one embodiment, the subject is at risk of developing an infectious disease. Examples of risks factor include, but are not limited to, genetic predisposition, or familial history of infectious diseases.
“Therapeutic agent”, “active pharmaceutical ingredient” and “active ingredient” refer to a compound for therapeutic use and relating to health. Especially, a therapeutic agent (e.g., a compound of the invention) may be indicated for treating a disease. An active ingredient may also be indicated for improving the therapeutic activity of another therapeutic agent.
“Therapeutically effective amount” (in short “effective amount”) refers to the amount of a therapeutic agent (e.g., a compound of the invention) that is sufficient to achieve the desired therapeutic, prophylactic or preventative effect in the patient to which/whom it is administered, without causing significant negative or adverse side effects to said patient. A therapeutically effective amount may be administered prior to the onset of the disease, disorder, or condition, for a prophylactic or preventive action. Alternatively, or additionally, the therapeutically effective amount may be administered after initiation of the disease, disorder, or condition, for a therapeutic action.
“Treating”, “treatment” or “alleviation” refers to both therapeutic treatment and prophylactic or preventative measures, wherein the object is to prevent or slow down (lessen) the targeted pathologic condition or disorder (i.e., a “disease”). Those in need of treatment include those already with the disease as well as those prone to have the disease or those in whom the condition or disease is to be prevented. A patient is successfully “treated” for a disease if, after receiving a therapeutic amount of a therapeutic agent (e.g., a compound according the present invention), the patient shows observable and/or measurable reduction in or absence of one or more of the following: reduction in the number of pathogens (e.g., infectious agents); reduction in the percent of total cells that are pathogenic; and/or relief to some extent, one or more of the symptoms associated with the specific disease; reduced morbidity and mortality, and improvement in quality of life issues. The above parameters for assessing successful treatment and improvement in the disease are readily measurable by routine procedures familiar to a physician.

### DETAILED DESCRIPTION

### Compound

The present invention relates to bile acid derivatives, which are obtainable from the coupling of a bile acid with a polyamine chain and with an amino acid derivative. In the invention, the amino acid may be natural or non-natural (e.g., synthetic or hemisynthetic). According to one embodiment, the amino acid is natural.

The present invention relates to a compound of formula (I)

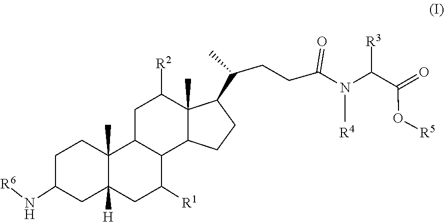

or a pharmaceutically acceptable salt and/or solvate thereof;
wherein
R¹ represents H, OH or SO₃H;
R² represents H, OH or SO₃H;
R³ represents H, C₁-C₈alkyl, C₆-C₁₀ aryl or C₆-C₁₀ aryl-C₁-C₈alkyl; wherein the alkyl group is optionally substituted by at least one OH, COOH, —C(O)NH₂, NH₂, —NH—C(═NH)—NH₂, imidazolyl, indolyl, SH, S—CH₃ or SeH;
wherein in the aryl or arylalkyl group, the aryl is optionally substituted by at least one OH;
R⁴ represents H, C₁-C₈alkyl or C₆-C₁₀ aryl;
or R³ and R⁴ form together with the nitrogen and carbon atoms to which they are attached a 5-membered heterocycloalkyl;
R⁵ represents H, C₁-C₈alkyl or C₆-C₁₀ aryl;
R⁶ represents —(CR⁷R⁸)ₘ—[X—(CR⁹R¹⁰)ₙ]ₚ—NR¹¹R¹² wherein
   R⁷, R⁸, R⁹ and R¹⁰ represent, independently at each occurrence, and each independently, H or C₁-C₈alkyl;
   R¹¹ and R¹² represent, each independently, H, C₁-C₈alkyl, or R¹¹ and R¹² form together with the nitrogen atom to which they are attached a 5- to 7-membered heterocyclyl optionally substituted by one to three R¹³; wherein R¹³ represents ═O or ═S;
   X represent, independently at each occurrence, —NR¹⁴— or a divalent 5- to
   7-membered heterocycloalkyl comprising at least one nitrogen atom; wherein R¹⁴ represents H, C₁-C₆ alkyl or —(CH₂)_{q}—NH₂; wherein q represents an integer ranging from 1 to 5;
m is an integer ranging from 2 to 10;
n is an integer ranging from 1 to 5; and
p is an integer ranging from 0 to 4.

The carbon atoms in formula (I) are numbered as shown on the following formula. Based on the above carbon atom numbering, in the compounds of formula (I) according to the invention, the polyamine chain is coupled in position 3 thereof and the amino acid is coupled in position 20 thereof.

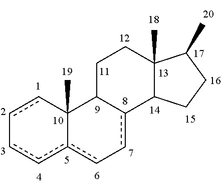

According to one embodiment, R¹ represents H or OH. According to one embodiment, R² represents H or OH. In one embodiment, R¹ represents H and R² represents OH. In one embodiment, R¹ represents OH and R² represents H. In one embodiment, R¹ and R² both represent H. In one embodiment, R¹ and R² both represent OH.

According to one embodiment, R¹ and R² represent H or OH and their stereochemical configuration as shown in any one of the following bile acids:

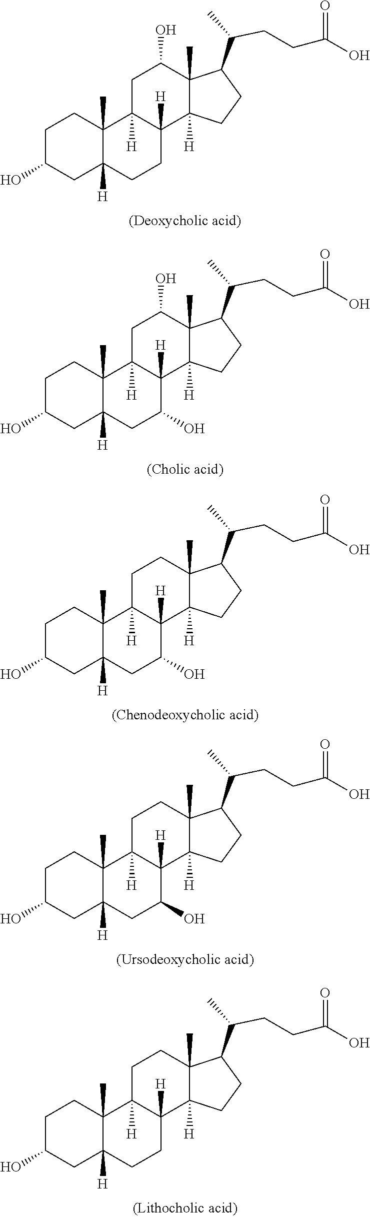

These bile acids may be used as starting material for manufacturing the compound of formula (I), as explained hereinafter. According to one embodiment, the bile acid is selected from deoxycholic acid, cholic acid, chenodeoxycholic acid and lithocholic acid.

According to one embodiment, R³ represents H, C₁-C₈alkyl, or C₆-C₁₀ aryl-C₁-C₈alkyl; wherein the alkyl group is optionally substituted by at least one OH, COOH, —C(O)NH₂, NH₂, —NH—C(═NH)—NH₂, imidazolyl, indolyl, SH, S—CH₃ or SeH; and wherein in the arylalkyl group, the aryl is optionally substituted by at least one OH. In one preferred embodiment, the C₁-C₈alkyl is unsubstituted.

According to one embodiment, R³ represents H. According to one embodiment, R³ represents C₁-C₈alkyl. In one embodiment, R³ represents C₁-C₆ alkyl. In one embodiment, R³ represents C₁-C₄ alkyl. In one embodiment, R³ represents methyl, propyl (e.g., i-propyl), or butyl (e.g., i-butyl or s-butyl). In one preferred embodiment, R³ represents methyl, propyl (e.g., i-propyl), or butyl (e.g., i-butyl or s-butyl).

According to one embodiment, R³ represents C₁-C₈alkyl substituted by at least one substituent selected from OH, COOH, —C(O)NH₂, NH₂, —NH—C(═NH)—NH₂, imidazolyl, indolyl, SH, S—CH₃ and SeH. In one embodiment, the alkyl is substituted by exactly one substituent selected from the preceding list. In one embodiment, the imidazolyl substituent is 4-imidazolyl, i.e., the imidazolyl is bound to the alkyl as in histidine (His) amino acid. In one embodiment, the indolyl substituent is 3-indolyl, i.e., the indolyl is bound to the alkyl as in tryptophan (Trp) amino acid.

According to one embodiment, R³ represents C₆-C₁₀ aryl-C₁-C₈ alkyl, wherein the aryl is optionally substituted by at least one OH. In one embodiment, the alkyl is C₁-C₆ alkyl. In one embodiment, the alkyl is C₁-C₄ alkyl. In one embodiment, the alkyl is C₁-C₂ alkyl. In one embodiment, the aryl is substituted by exactly one OH. In one embodiment, the aryl is phenyl. In one embodiment, R³ represents phenyl-(CH₂)₂— or benzyl (i.e., phenyl-CH₂-), wherein the phenyl is optionally substituted by at least one OH. In one embodiment, the phenyl is substituted by exactly one OH. In one preferred embodiment, R³ represents benzyl or para-hydroxybenzyl.

According to one embodiment, R⁴ represents H.

According to one embodiment, R³ and R⁴ form together with the nitrogen and carbon atoms to which they are attached a 5-membered heterocycloalkyl. According to one embodiment, R³ and R⁴ form together with the nitrogen and carbon atoms to which they are attached a 5-membered heterocycloalkyl comprising exactly one nitrogen atom. In one embodiment, R³ and R⁴ form together with the nitrogen and carbon atoms to which they are attached a divalent pyrrolidine (e.g., a divalent 1,2-pyrrolidine).

According to one embodiment, R⁵ represents H. According to one embodiment, R⁵ represents C₁-C₈alkyl. In one embodiment, R⁵ represents C₁-C₆ alkyl. In one embodiment, R⁵ represents C₁-C₄ alkyl. In one preferred embodiment, R⁵ represents methyl.

According to one preferred embodiment, R⁷ and R¹ represent both H. According to one embodiment, R⁹ and R¹⁰ represent both H. In one embodiment, R⁷, R¹, R⁹ and R¹⁰ represent H.

According to one preferred embodiment, R¹¹ and R¹² represent both H. In one preferred embodiment, R⁷ and R⁸ represent both H and R¹¹ and R¹² represent both H.

According to one embodiment, R¹¹ and R¹² form together with the nitrogen atom to which they are attached a 5- or 6-membered heterocyclyl optionally substituted by one to three R¹³. According to one embodiment, R¹¹ and R¹² form together with the nitrogen atom to which they are attached a 5- to 7-membered heterocycloalkyl optionally substituted by one to three R¹³. In one embodiment, R¹¹ and R¹² form together with the nitrogen atom to which they are attached a 5- or 6-membered heterocycloalkyl optionally substituted by one to three R¹³. In one embodiment, the heterocyclyl is unsubstituted by R¹³ or substituted by exactly one R¹³

According to one embodiment, X represents —NR¹⁴— wherein R¹⁴ is as defined herein. In one embodiment, X represents —NH—, i.e., R¹⁴ represents H. In one embodiment, R¹⁴ represents C₁-C₆ alkyl. In one embodiment, R¹⁴ represents C₁-C₄ alkyl. In one embodiment, R¹⁴ represents methyl. In one embodiment, R¹⁴ represents methyl. In one embodiment, R¹⁴ represents —(CH₂)_{q}—NH₂; wherein q ranges from 1 to 5. In one embodiment, q ranges from 2 to 4. In one embodiment, q is 3.

According to one embodiment, X represents a divalent 5- to 7-membered heterocycloalkyl comprising at least one nitrogen atom. In one embodiment, the heterocycloalkyl is 5- or 6-membered. In one embodiment, the heterocycloalkyl is 6-membered. In one embodiment, the heterocycloalkyl comprises at least two nitrogen atoms. In one embodiment, the heterocycloalkyl comprises only nitrogen atoms as heteroatoms. In one embodiment, X represents a divalent piperazine (e.g., a divalent 1,4-piperazine).

According to one embodiment, m ranges from 2 to 6. In one embodiment, m ranges from 2 to 4. In one embodiment, m is 2 or 3. According to one embodiment, n ranges from 2 to 5. In one embodiment, n is 2, 3 or 4. According to one embodiment, p ranges from 0 to 3. In one embodiment, p is 1 or 2.

According to one embodiment, R⁶ represents H₂N—(CH₂)ᵣ—, wherein r is an integer ranging from 1 to 12. In one embodiment, r ranges from 1 to 10. In one embodiment, r ranges from 1 to 6.

According to one embodiment, R⁶ represents any one of the following formulae.

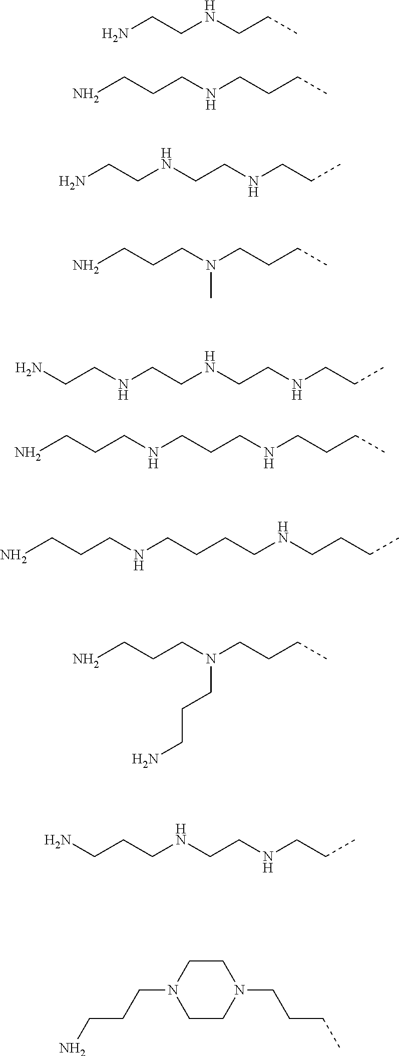

wherein the dotted bond indicates the point of attachment of R⁶ to the nitrogen atom.

According to one embodiment, R⁶ represents any one of the following formulae.

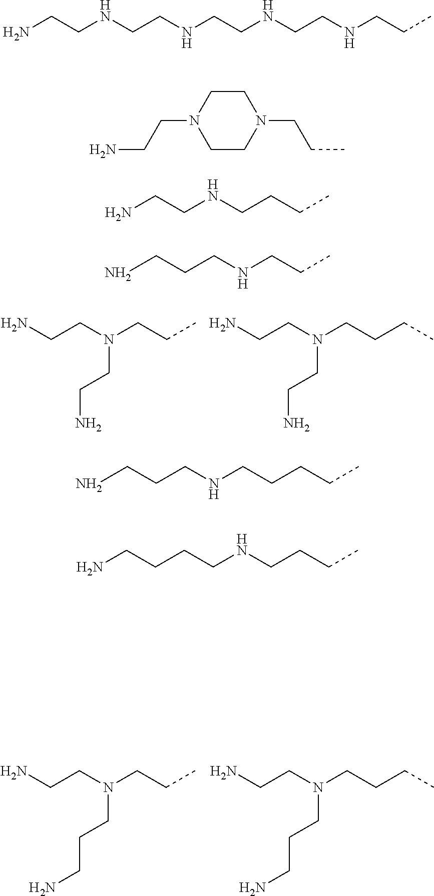

wherein the dotted bond indicates the point of attachment of R⁶ to the nitrogen atom.

According to one embodiment, the compound of formula (I) is selected from the compounds of Table 1 below, and pharmaceutically acceptable salts and/or solvates thereof.

TABLE 1

| Cpd | Structure | Name |
|---|---|---|
| 001 | 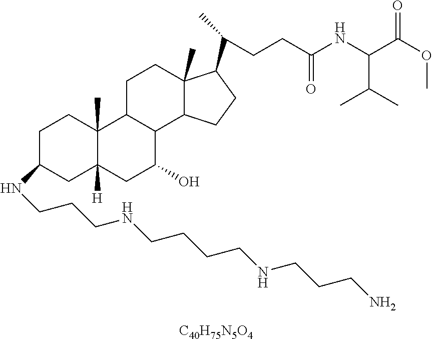 [SMILES: COC(=O)C(NC(=O)CC[C@@H](C)[C@H]1CCC2C3C(CC[C@@]21C)[C@@]1(C)CC[C@H](NCCCNCCCCNCCCN)C[C@H]1C[C@H]3O)C(C)C ; "C40H75N5O4"] | methyl ((4R)-4- ((3S,5S,7R,10S,13R,17R)-3- ((3-((4-((3- aminopropyl)amino)butyl) amino)propyl)amino)-7- hydroxy-10,13- dimethylhexadecahydro-1H- cyclopenta[a]phenanthren-17- yl)pentanoyl)valinate |
| 002 | 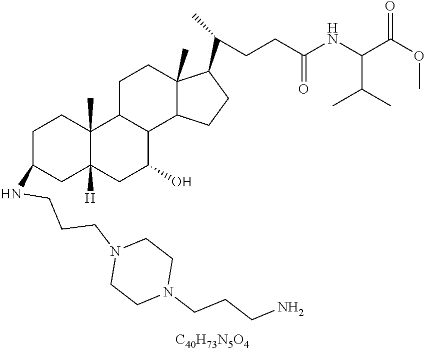 [SMILES: COC(=O)C(NC(=O)CC[C@@H](C)[C@H]1CCC2C3C(CC[C@@]21C)[C@@]1(C)CC[C@H](NCCCN2CCN(CCCN)CC2)C[C@H]1C[C@H]3O)C(C)C ; "C40H73N5O4"] | methyl ((4R)-4- ((3S,5S,7R,10S,13R,17R)-3- ((3-(4-(3- aminopropyl)piperazin-1- yl)propyl)amino)-7-hydroxy- 10,13- dimethylhexadecahydro-1H- cyclopenta[a]phenanthren-17- yl)pentanoyl)valinate |
| 003 | 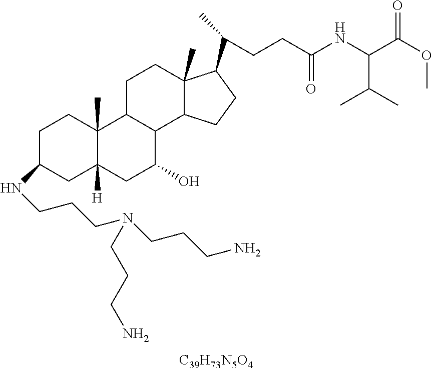 [SMILES: COC(=O)C(NC(=O)CC[C@@H](C)[C@H]1CCC2C3C(CC[C@@]21C)[C@@]1(C)CC[C@H](NCCCN(CCCN)CCCN)C[C@H]1C[C@H]3O)C(C)C ; "C39H73N5O4"] | methyl ((4R)-4- ((3S,5S,7R,10S,13R,17R)-3- ((3-(bis(3- aminopropyl)amino)propyl) amino)-7-hydroxy-10,13- dimethylhexadecahydro-1H- cyclopenta[a]phenanthren-17- yl)pentanoyl)valinate |
| 004 | 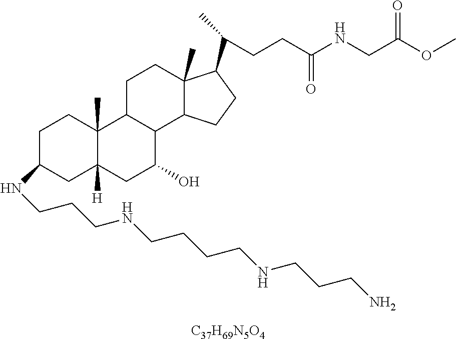 [SMILES: COC(=O)CNC(=O)CC[C@@H](C)[C@H]1CCC2C3C(CC[C@@]21C)[C@@]1(C)CC[C@H](NCCCNCCCCNCCCN)C[C@H]1C[C@H]3O ; "C37H69N5O4"] | methyl ((4R)-4- ((3S,5S,7R,10S,13R,17R)-3- ((3-((4-((3- aminopropyl)amino)butyl) amino)propyl)amino)-7- hydroxy-10,13- dimethylhexadecahydro-1H- cyclopenta[a]phenanthren-17- yl)pentanoyl)glycinate |
| 005 | 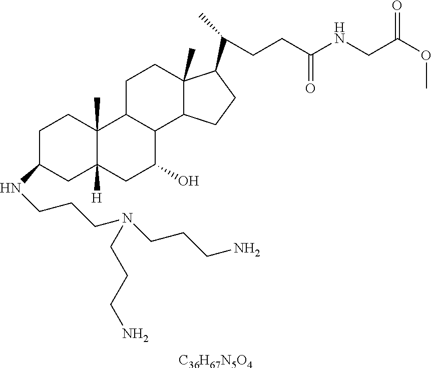 [SMILES: COC(=O)CNC(=O)CC[C@@H](C)[C@H]1CCC2C3C(CC[C@@]21C)[C@@]1(C)CC[C@H](NCCCN(CCCN)CCCN)C[C@H]1C[C@H]3O ; "C36H67N5O4"] | methyl ((4R)-4- ((3S,5S,7R,10S,13R,17R)-3- ((3-(bis(3- aminopropyl)amino)propyl) amino)-7-hydroxy-10,13- dimethylhexadecahydro-1H- cyclopenta[a]phenanthren-17- yl)pentanoyl)glycinate |
| 006 | 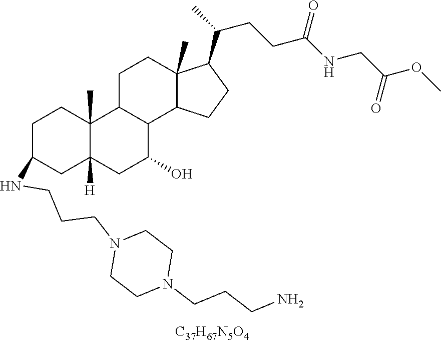 [SMILES: COC(=O)CNC(=O)CC[C@@H](C)[C@H]1CCC2C3C(CC[C@@]21C)[C@@]1(C)CC[C@H](NCCCN2CCN(CCCN)CC2)C[C@H]1C[C@H]3O ; "C37H67N5O4"] | methyl ((4R)-4- ((3S,5S,7R,10S,13R,17R)-3- ((3-(4-(3- aminopropyl)piperazin-1- yl)propyl)amino)-7-hydroxy- 10,13- dimethylhexadecahydro-1H- cyclopenta[a]phenanthren-17- yl)pentanoyl)glycinate |
| 007 | 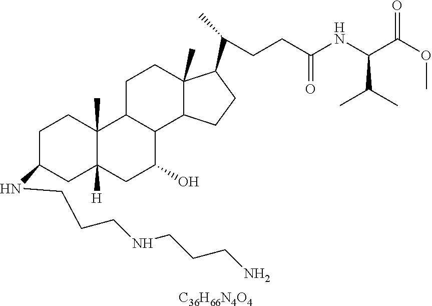 | methyl ((4R)-4- ((3S,5S,7R,10S,13R,17R)-3- ((3-((3- aminopropyl)amino)propyl) amino)-7-hydroxy-10,13- dimethylhexadecahydro-1H- cyclopenta[a]phenanthren-17- yl)pentanoyl)-D-valinate |
| 008 | 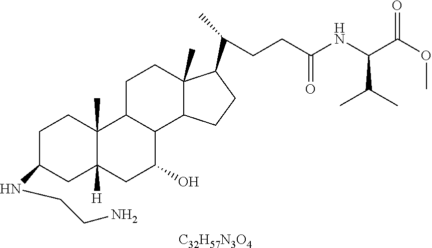 [SMILES: COC(=O)[C@H](NC(=O)CC[C@@H](C)[C@H]1CCC2C3C(CC[C@@]21C)[C@@]1(C)CC[C@H](NCCN)C[C@H]1C[C@H]3O)C(C)C ; "C32H57N3O4"] | methyl ((4R)-4- ((3S,5S,7R,10S,13R,17R)-3- ((2-aminoethyl)amino)-7- hydroxy-10,13- dimethylhexadecahydro-1H- cyclopenta[a]phenanthren-17- yl)pentanoyl)-D-valinate |
| 009 | 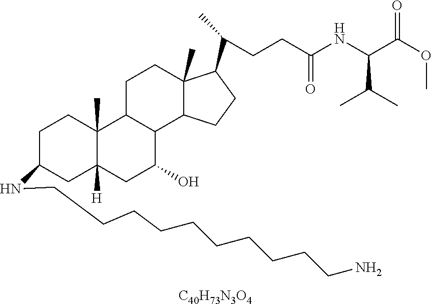 [SMILES: COC(=O)[C@H](NC(=O)CC[C@@H](C)[C@H]1CCC2C3C(CC[C@@]21C)[C@@]1(C)CC[C@H](NCCCCCCCCCCN)C[C@H]1C[C@H]3O)C(C)C ; "C40H73N3O4"] | methyl ((4R)-4- ((3S,5S,7R,10S,13R,17R)-3- ((10-aminodecyl)amino)-7- hydroxy-10,13- dimethylhexadecahydro-1H- cyclopenta[a]phenanthren-17- yl)pentanoyl)-D-valinate |
| 010 | 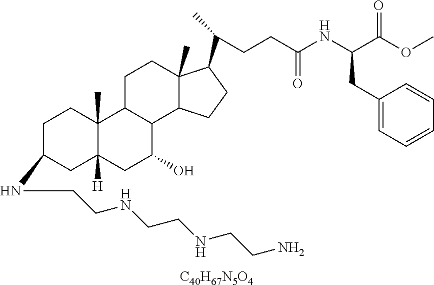 [SMILES: COC(=O)[C@@H](Cc1ccccc1)NC(=O)CC[C@@H](C)[C@H]1CCC2C3C(CC[C@@]21C)[C@@]1(C)CC[C@H](NCCNCCNCCN)C[C@H]1C[C@H]3O ; "C40H67N5O4"] | methyl ((4R)-4- ((3S,5S,7R,10S,13R,17R)-3- ((2-((2-((2- aminoethyl)amino)ethyl) amino)ethyl)amino)-7- hydroxy-10,13- dimethylhexadecahydro-1H- cyclopenta[a]phenanthren-17- yl)pentanoyl)-D- phenylalaninate |
| 011 | 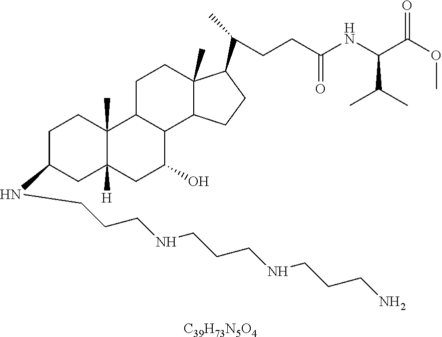 [SMILES: COC(=O)[C@H](NC(=O)CC[C@@H](C)[C@H]1CCC2C3C(CC[C@@]21C)[C@@]1(C)CC[C@H](NCCCNCCCNCCCN)C[C@H]1C[C@H]3O)C(C)C ; "C39H73N5O4"] | methyl ((4R)-4- ((3S,5S,7R,10S,13R,17R)-3- ((3-((3-((3- aminopropyl)amino)propyl) amino)propyl)amino)-7- hydroxy-10,13- dimethylhexadecahydro-1H- cyclopenta[a]phenanthren-17- yl)pentanoyl)-D-valinate |
| 012 | 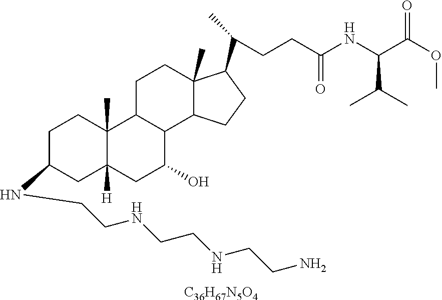 [SMILES: COC(=O)[C@H](NC(=O)CC[C@@H](C)[C@H]1CCC2C3C(CC[C@@]21C)[C@@]1(C)CC[C@H](NCCNCCNCCN)C[C@H]1C[C@H]3O)C(C)C ; "C36H67N5O4"] | methyl ((4R)-4- ((3S,5S,7R,10S,13R,17R)-3- ((2-((2-((2- aminoethyl)amino)ethyl) amino)ethyl)amino)-7- hydroxy-10,13- dimethylhexadecahydro-1H- cyclopenta[a]phenanthren-17- yl)pentanoyl)-D-valinate |
| 013 | 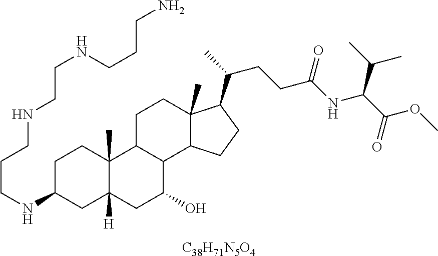 [SMILES: COC(=O)[C@@H](NC(=O)CC[C@@H](C)[C@H]1CCC2C3C(CC[C@@]21C)[C@@]1(C)CC[C@H](NCCCNCCNCCCN)C[C@H]1C[C@H]3O)C(C)C ; "C38H71N5O4"] | methyl ((4R)-4- ((3S,5S,7R,10S,13R,17R)-3- ((3-((2-((3- aminopropyl)amino)ethyl) amino)propyl)amino)-7- hydroxy-10,13- dimethylhexadecahydro-1H- cyclopenta[a]phenanthren-17- yl)pentanoyl)-L-valinate |
| 014 | 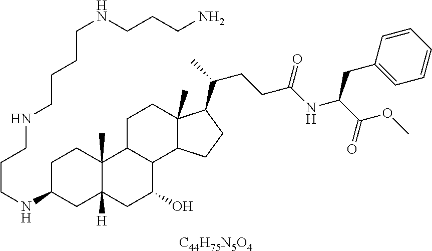 [SMILES: COC(=O)[C@H](Cc1ccccc1)NC(=O)CC[C@@H](C)[C@H]1CCC2C3C(CC[C@@]21C)[C@@]1(C)CC[C@H](NCCCNCCCCNCCCN)C[C@H]1C[C@H]3O ; "C44H75N5O4"] | methyl ((4R)-4- ((3S,5S,7R,10S,13R,17R)-3- ((3-((4-((3- aminopropyl)amino)butyl) amino)propyl)amino)-7- hydroxy-10,13- dimethylhexadecahydro-1H- cyclopenta[a]phenanthren-17- yl)pentanoyl)-L- phenylalaninate |
| 015 | 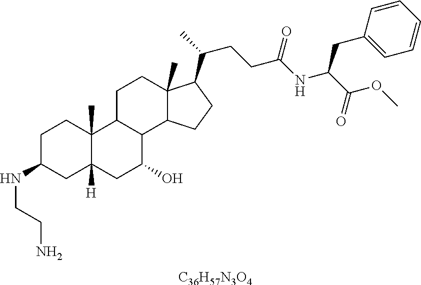 [SMILES: COC(=O)[C@H](Cc1ccccc1)NC(=O)CC[C@@H](C)[C@H]1CCC2C3C(CC[C@@]21C)[C@@]1(C)CC[C@H](NCCN)C[C@H]1C[C@H]3O ; "C36H57N3O4"] | methyl ((4R)-4- ((3S,5S,7R,10S,13R,17R)-3- ((2-aminoethyl)amino)-7- hydroxy-10,13- dimethylhexadecahydro-1H- cyclopenta[a]phenanthren-17- yl)pentanoyl)-L- phenylalaninate |
| 016 | 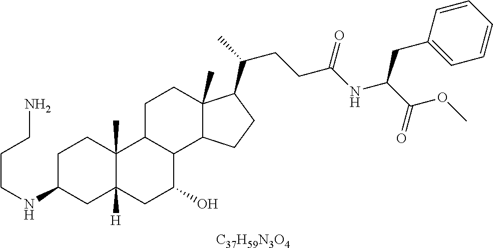 [SMILES: COC(=O)[C@H](Cc1ccccc1)NC(=O)CC[C@@H](C)[C@H]1CCC2C3C(CC[C@@]21C)[C@@]1(C)CC[C@H](NCCCN)C[C@H]1C[C@H]3O ; "C37H59N3O4"] | methyl ((4R)-4- ((3S,5S,7R,10S,13R,17R)-3- ((3-aminopropyl)amino)-7- hydroxy-10,13- dimethylhexadecahydro-1H- cyclopenta[a]phenanthren-17- yl)pentanoyl)-L- phenylalaninate |
| 018 | 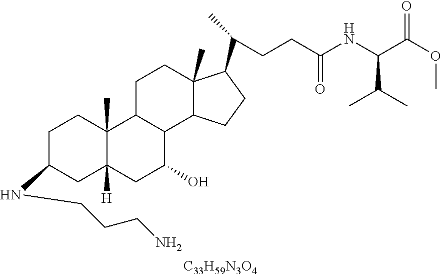 [SMILES: COC(=O)[C@H](NC(=O)CC[C@@H](C)[C@H]1CCC2C3C(CC[C@@]21C)[C@@]1(C)CC[C@H](NCCCN)C[C@H]1C[C@H]3O)C(C)C ; "C33H59N3O4"] | methyl ((4R)-4- ((3S,5S,7R,10S,13R,17R)-3- ((3-aminopropyl)amino)-7- hydroxy-10,13- dimethylhexadecahydro-1H- cyclopenta[a]phenanthren-17- yl)pentanoyl)-D-valinate |
| 020 | 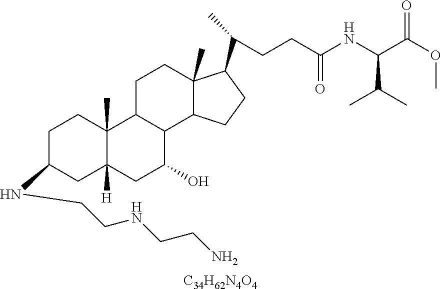 | methyl ((4R)-4- ((3S,5S,7R,10S,13R,17R)-3- ((2-((2- aminoethyl)amino)ethyl) amino)-7-hydroxy-10,13- dimethylhexadecahydro-1H- cyclopenta[a]phenanthren-17- yl)pentanoyl)-D-valinate |
| 022 | 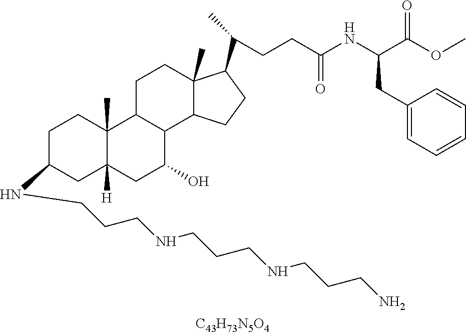 [SMILES: COC(=O)[C@@H](Cc1ccccc1)NC(=O)CC[C@@H](C)[C@H]1CCC2C3C(CC[C@@]21C)[C@@]1(C)CC[C@H](NCCCNCCCNCCCN)C[C@H]1C[C@H]3O ; "C43H73N5O4"] | methyl ((4R)-4- ((3S,5S,7R,10S,13R,17R)-3- ((3-((3-((3- aminopropyl)amino)propyl) amino)propyl)amino)-7- hydroxy-10,13- dimethylhexadecahydro-1H- cyclopenta[a]phenanthren-17- yl)pentanoyl)-D- phenylalaninate |
| 023 | 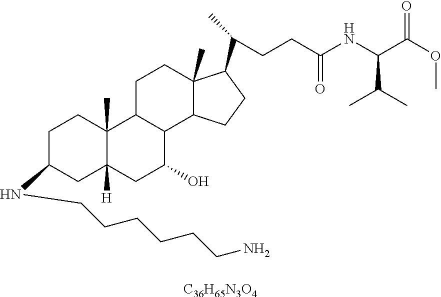 [SMILES: COC(=O)[C@H](NC(=O)CC[C@@H](C)[C@H]1CCC2C3C(CC[C@@]21C)[C@@]1(C)CC[C@H](NCCCCCCN)C[C@H]1C[C@H]3O)C(C)C ; "C36H65N3O4"] | methyl ((4R)-4- ((3S,5S,7R,10S,13R,17R)-3- ((6-aminohexyl)amino)-7- hydroxy-10,13- dimethylhexadecahydro-1H- cyclopenta[a]phenanthren-17- yl)pentanoyl)-D-valinate |
| 024 | 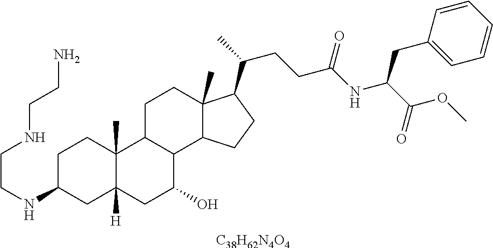 | methyl ((4R)-4- ((3S,5S,7R,10S,13R,17R)-3- ((2-((2- aminoethyl)amino)ethyl) amino)-7-hydroxy-10,13- dimethylhexadecahydro-1H- cyclopenta[a]phenanthren-17- yl)pentanoyl)-L- phenylalaninate |
| 025 | 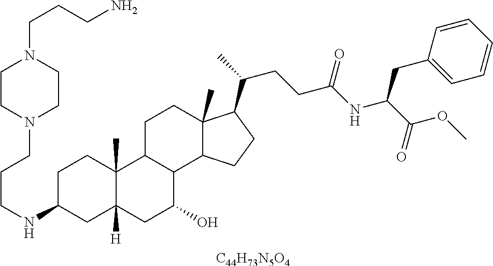 [SMILES: COC(=O)[C@H](Cc1ccccc1)NC(=O)CC[C@@H](C)[C@H]1CCC2C3C(CC[C@@]21C)[C@@]1(C)CC[C@H](NCCCN2CCN(CCCN)CC2)C[C@H]1C[C@H]3O ; "C44H73N5O4"] | methyl ((4R)-4- ((3S,5S,7R,10S,13R,17R)-3- ((3-(4-(3- aminopropyl)piperazin-1- yl)propyl)amino)-7-hydroxy- 10,13- dimethylhexadecahydro-1H- cyclopenta[a]phenanthren-17- yl)pentanoyl)-L- phenylalaninate |
| 026 | 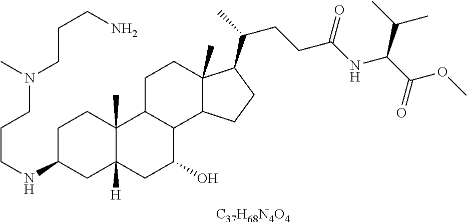 | methyl ((4R)-4- ((3S,5S,7R,10S,13R,17R)-3- ((3-((3- aminopropyl)(methyl)amino) propyl)amino)-7-hydroxy- 10,13- dimethylhexadecahydro-1H- cyclopenta[a]phenanthren-17- yl)pentanoyl)-L-valinate |
| 028 | 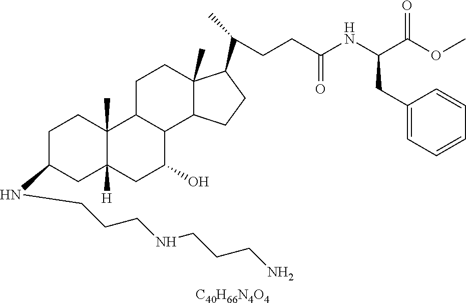 | methyl ((4R)-4- ((3S,5S,7R,10S,13R,17R)-3- ((3-((3- aminopropyl)amino)propyl) amino)-7-hydroxy-10,13- dimethylhexadecahydro-1H- cyclopenta[a]phenanthren-17- yl)pentanoyl)-D- phenylalaninate |
| 029 | 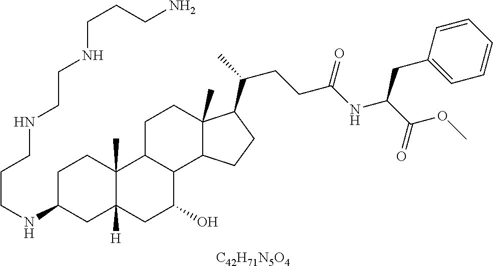 [SMILES: COC(=O)[C@H](Cc1ccccc1)NC(=O)CC[C@@H](C)[C@H]1CCC2C3C(CC[C@@]21C)[C@@]1(C)CC[C@H](NCCCNCCNCCCN)C[C@H]1C[C@H]3O ; "C42H71N5O4"] | methyl ((4R)-4- ((3S,5S,7R,10S,13R,17R)-3- ((3-((2-((3- aminopropyl)amino)ethyl) amino)propyl)amino)-7- hydroxy-10,13- dimethylhexadecahydro-1H- cyclopenta[a]phenanthren-17- yl)pentanoyl)-L- phenylalaninate |
| 030 | 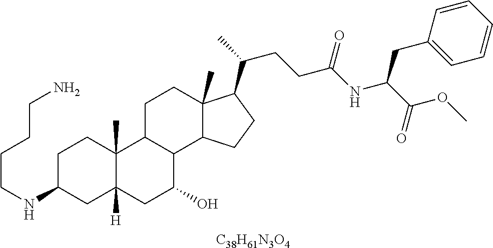 [SMILES: COC(=O)[C@H](Cc1ccccc1)NC(=O)CC[C@@H](C)[C@H]1CCC2C3C(CC[C@@]21C)[C@@]1(C)CC[C@H](NCCCCN)C[C@H]1C[C@H]3O ; "C38H61N3O4"] | methyl ((4R)-4- ((3S,5S,7R,10S,13R,17R)-3- ((4-aminobutyl)amino)-7- hydroxy-10,13- dimethylhexadecahydro-1H- cyclopenta[a]phenanthren-17- yl)pentanoyl)-L- phenylalaninate |
| 031 | 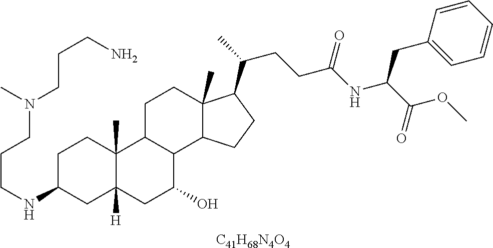 | methyl ((4R)-4- ((3S,5S,7R,10S,13R,17R)-3- ((3-((3- aminopropyl)(methyl)amino) propyl)amino)-7-hydroxy- 10,13- dimethylhexadecahydro-1H- cyclopenta[a]phenanthren-17- yl)pentanoyl)-L- phenylalaninate |
| 032 | 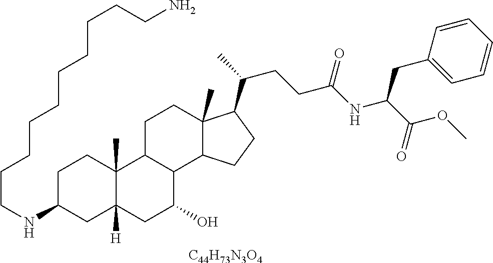 [SMILES: COC(=O)[C@H](Cc1ccccc1)NC(=O)CC[C@@H](C)[C@H]1CCC2C3C(CC[C@@]21C)[C@@]1(C)CC[C@H](NCCCCCCCCCCN)C[C@H]1C[C@H]3O ; "C44H73N3O4"] | methyl ((4R)-4- ((3S,5S,7R,10S,13R,17R)-3- ((10-aminodecyl)amino)-7- hydroxy-10,13- dimethylhexadecahydro-1H- cyclopenta[a]phenanthren-17- yl)pentanoyl)-L- phenylalaninate |
| 034 | 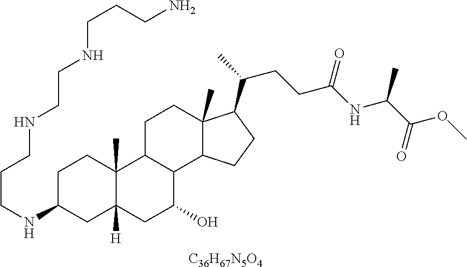 [SMILES: COC(=O)[C@H](C)NC(=O)CC[C@@H](C)[C@H]1CCC2C3C(CC[C@@]21C)[C@@]1(C)CC[C@H](NCCCNCCNCCCN)C[C@H]1C[C@H]3O ; "C36H67N5O4"] | methyl ((4R)-4- ((3S,5S,7R,10S,13R,17R)-3- ((3-((2-((3- aminopropyl)amino)ethyl) amino)propyl)amino)-7- hydroxy-10,13- dimethylhexadecahydro-1H- cyclopenta[a]phenanthren-17- yl)pentanoyl)-L-alaninate |
| 035 | 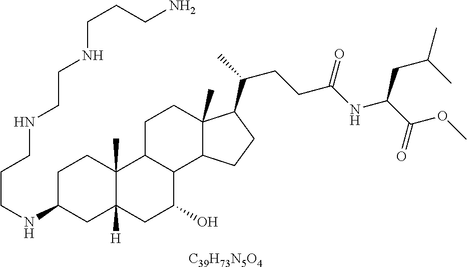 [SMILES: COC(=O)[C@H](CC(C)C)NC(=O)CC[C@@H](C)[C@H]1CCC2C3C(CC[C@@]21C)[C@@]1(C)CC[C@H](NCCCNCCNCCCN)C[C@H]1C[C@H]3O ; "C39H73N5O4"] | methyl ((4R)-4- ((3S,5S,7R,10S,13R,17R)-3- ((3-((2-((3- aminopropyl)amino)ethyl) amino)propyl)amino)-7- hydroxy-10,13- dimethylhexadecahydro-1H- cyclopenta[a]phenanthren-17- yl)pentanoyl)-L-leucinate |
| 036 | 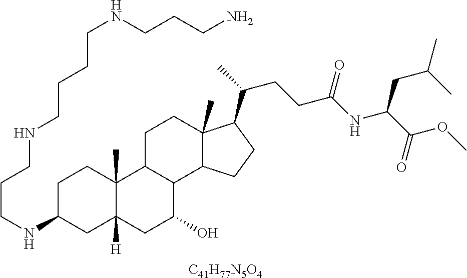 [SMILES: COC(=O)[C@H](CC(C)C)NC(=O)CC[C@@H](C)[C@H]1CCC2C3C(CC[C@@]21C)[C@@]1(C)CC[C@H](NCCCNCCCCNCCCN)C[C@H]1C[C@H]3O ; "C41H77N5O4"] | methyl ((4R)-4- ((3S,5S,7R,10S,13R,17R)-3- ((3-((4-((3- aminopropyl)amino)butyl) amino)propyl)amino)-7- hydroxy-10,13- dimethylhexadecahydro-1H- cyclopenta[a]phenanthren-17- yl)pentanoyl)-L-leucinate |
| 037 | 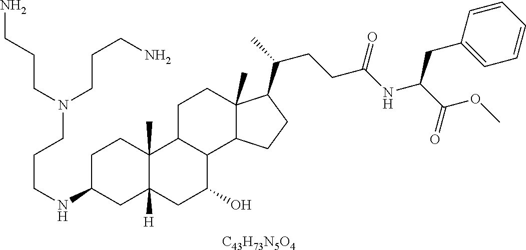 [SMILES: COC(=O)[C@H](Cc1ccccc1)NC(=O)CC[C@@H](C)[C@H]1CCC2C3C(CC[C@@]21C)[C@@]1(C)CC[C@H](NCCCN(CCCN)CCCN)C[C@H]1C[C@H]3O ; "C43H73N5O4"] | methyl ((4R)-4- ((3S,5S,7R,10S,13R,17R)-3- ((3-(bis(3- aminopropyl)amino)propyl) amino)-7-hydroxy-10,13- dimethylhexadecahydro-1H- cyclopenta[a]phenanthren-17- yl)pentanoyl)-L- phenylalaninate |
| 038 | 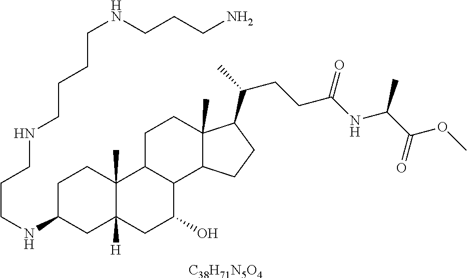 [SMILES: COC(=O)[C@H](C)NC(=O)CC[C@@H](C)[C@H]1CCC2C3C(CC[C@@]21C)[C@@]1(C)CC[C@H](NCCCNCCCCNCCCN)C[C@H]1C[C@H]3O ; "C38H71N5O4"] | methyl ((4R)-4- ((3S,5S,7R,10S,13R,17R)-3- ((3-((4-((3- aminopropyl)amino)butyl) amino)propyl)amino)-7- hydroxy-10,13- dimethylhexadecahydro-1H- cyclopenta[a]phenanthren-17- yl)pentanoyl)-L-alaninate |
| 039 | 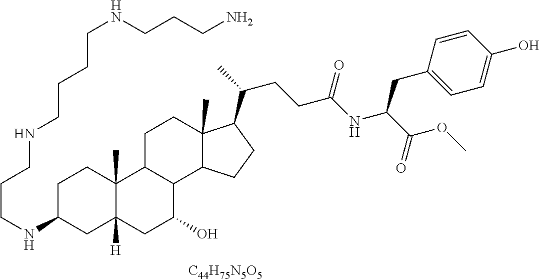 [SMILES: COC(=O)[C@H](Cc1ccc(O)cc1)NC(=O)CC[C@@H](C)[C@H]1CCC2C3C(CC[C@@]21C)[C@@]1(C)CC[C@H](NCCCNCCCCNCCCN)C[C@H]1C[C@H]3O ; "C44H75N5O5"] | methyl ((4R)-4- ((3S,5S,7R,10S,13R,17R)-3- ((3-((4-((3- aminopropyl)amino)butyl) amino)propyl)amino)-7- hydroxy-10,13- dimethylhexadecahydro-1H- cyclopenta[a]phenanthren-17- yl)pentanoyl)-L-tyrosinate |
| 040 | 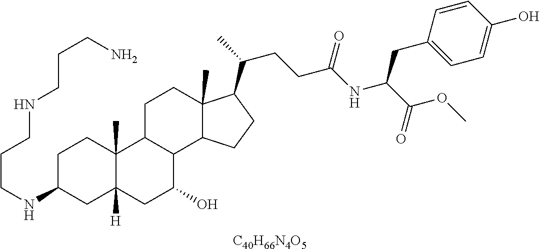 [SMILES: COC(=O)[C@H](Cc1ccc(O)cc1)NC(=O)CC[C@@H](C)[C@H]1CCC2C3C(CC[C@@]21C)[C@@]1(C)CC[C@H](NCCCNCCCN)C[C@H]1C[C@H]3O ; "C40H66N4O5"] | methyl ((4R)-4- ((3S,5S,7R,10S,13R,17R)-3- ((3-((3- aminopropyl)amino)propyl) amino)-7-hydroxy-10,13- dimethylhexadecahydro-1H- cyclopenta[a]phenanthren-17- yl)pentanoyl)-L-tyrosinate |
| 041 | 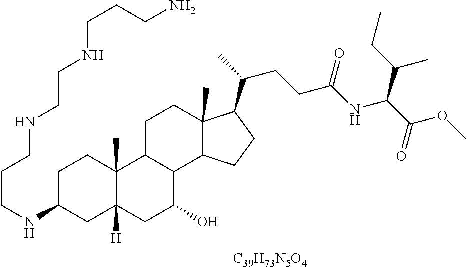 [SMILES: CCC(C)[C@H](NC(=O)CC[C@@H](C)[C@H]1CCC2C3C(CC[C@@]21C)[C@@]1(C)CC[C@H](NCCCNCCNCCCN)C[C@H]1C[C@H]3O)C(=O)OC ; "C39H73N5O4"] | methyl (2S,3S)-2-((4R)-4- ((3S,5S,7R,10S,13R,17R)-3- ((3-((2-((3- aminopropyl)amino)ethyl) amino)propyl)amino)-7- hydroxy-10,13- dimethylhexadecahydro-1H- cyclopenta[a]phenanthren-17- yl)pentanamido)-3- methylpentanoate |
| 042 | 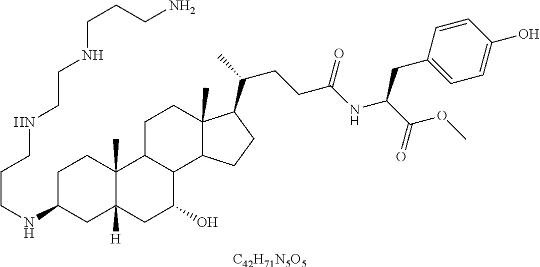 [SMILES: COC(=O)[C@H](Cc1ccc(O)cc1)NC(=O)CC[C@@H](C)[C@H]1CCC2C3C(CC[C@@]21C)[C@@]1(C)CC[C@H](NCCCNCCNCCCN)C[C@H]1C[C@H]3O ; "C42H71N5O5"] | methyl ((4R)-4- ((3S,5S,7R,10S,13R,17R)-3- ((3-((2-((3- aminopropyl)amino)ethyl) amino)propyl)amino)-7- hydroxy-10,13- dimethylhexadecahydro-1H- cyclopenta[a]phenanthren-17- yl)pentanoyl)-L-tyrosinate |
| 043 | 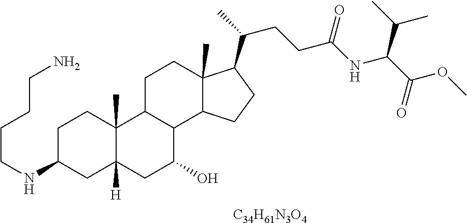 [SMILES: COC(=O)[C@@H](NC(=O)CC[C@@H](C)[C@H]1CCC2C3C(CC[C@@]21C)[C@@]1(C)CC[C@H](NCCCCN)C[C@H]1C[C@H]3O)C(C)C ; "C34H61N3O4"] | methyl ((4R)-4- ((3S,5S,7R,10S,13R,17R)-3- ((4-aminobutyl)amino)-7- hydroxy-10,13- dimethylhexadecahydro-1H- cyclopenta[a]phenanthren-17- yl)pentanoyl)-L-valinate |
| 044 | 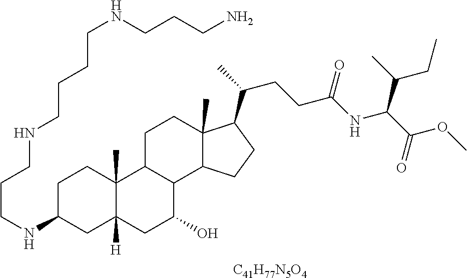 [SMILES: CCC(C)[C@H](NC(=O)CC[C@@H](C)[C@H]1CCC2C3C(CC[C@@]21C)[C@@]1(C)CC[C@H](NCCCNCCCCNCCCN)C[C@H]1C[C@H]3O)C(=O)OC ; "C41H77N5O4"] | methyl (2S,3R)-2-((4R)-4- ((3S,5S,7R,10S,13R,17R)-3- ((3-((4-((3- aminopropyl)amino)butyl) amino)propyl)amino)-7- hydroxy-10,13- dimethylhexadecahydro-1H- cyclopenta[a]phenanthren-17- yl)pentanamido)-3- methylpentanoate |
| 045 | 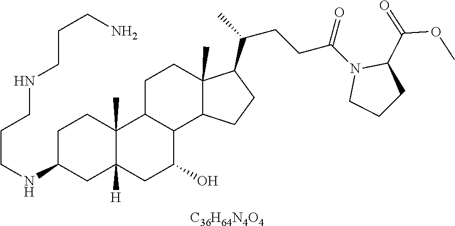 | methyl ((4R)-4- ((3S,5S,7R,10S,13R,17R)-3- ((3-((3- aminopropyl)amino)propyl) amino)-7-hydroxy-10,13- dimethylhexadecahydro-1H- cyclopenta[a]phenanthren-17- yl)pentanoyl)-D-prolinate |
| 046 | 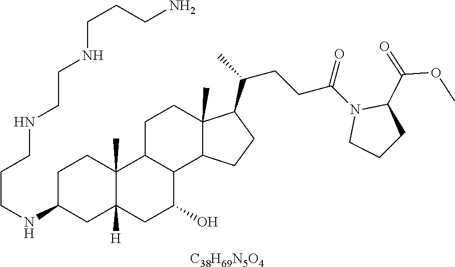 [SMILES: COC(=O)[C@H]1CCCN1C(=O)CC[C@@H](C)[C@H]1CCC2C3C(CC[C@@]21C)[C@@]1(C)CC[C@H](NCCCNCCNCCCN)C[C@H]1C[C@H]3O ; "C38H69N5O4"] | methyl ((4R)-4- ((3S,5S,7R,10S,13R,17R)-3- ((3-((2-((3- aminopropyl)amino)ethyl) amino)propyl)amino)-7- hydroxy-10,13- dimethylhexadecahydro-1H- cyclopenta[a]phenanthren-17- yl)pentanoyl)-D-prolinate |
| 049 | 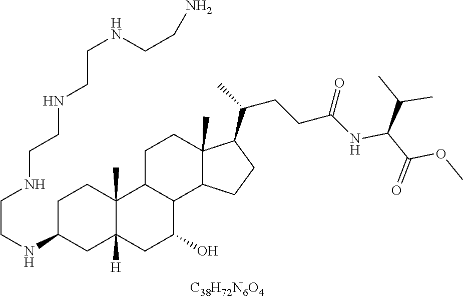 | methyl ((4R)-4- ((3S,5S,7R,10S,13R,17R)-3- ((2-((2-((2-((2- aminoethyl)amino)ethyl) amino)ethyl)amino)ethyl) amino)-7-hydroxy-10,13- dimethylhexadecahydro-1H- cyclopenta[a]phenanthren-17- yl)pentanoyl)-L-valinate |
| 050 | 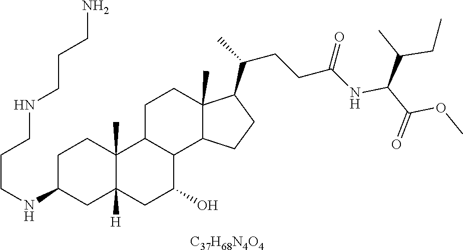 | methyl (2S,3R)-2-((4R)-4- ((3S,5S,7R,10S,13R,17R)-3- ((3-((3- aminopropyl)amino)propyl) amino)-7-hydroxy-10,13- dimethylhexadecahydro-1H- cyclopenta[a]phenanthren-17- yl)pentanamido)-3- methylpentanoate |
| 051 | 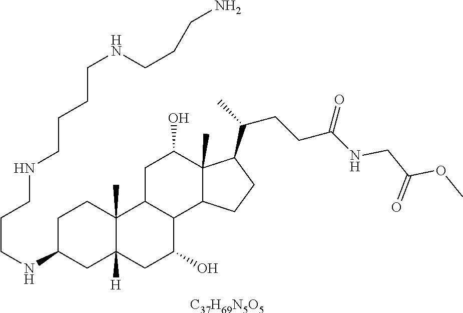 [SMILES: COC(=O)CNC(=O)CC[C@@H](C)[C@H]1CCC2C3C(C[C@H](O)[C@@]21C)[C@@]1(C)CC[C@H](NCCCNCCCCNCCCN)C[C@H]1C[C@H]3O ; "C37H69N5O5"] | methyl ((4R)-4- ((3S,5S,7R,10S,12S,13R,17R)- 3-((3-((4-((3- aminopropyl)amino)butyl) amino)propyl)amino)-7,12- dihydroxy-10,13- dimethylhexadecahydro-1H- cyclopenta[a]phenanthren-17- yl)pentanoyl)glycinate |
| 052 | 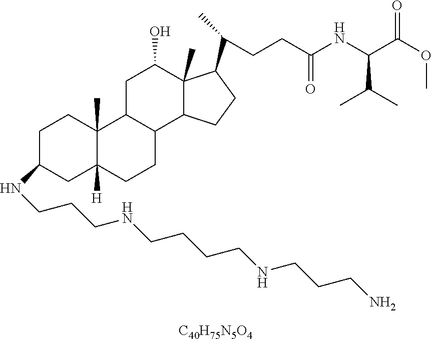 [SMILES: COC(=O)[C@H](NC(=O)CC[C@@H](C)[C@H]1CCC2C3CC[C@@H]4C[C@@H](NCCCNCCCCNCCCN)CC[C@]4(C)C3C[C@H](O)[C@@]21C)C(C)C ; "C40H75N5O4"] | methyl ((4R)-4- ((3S,5R,10S,12S,13R,17R)-3- ((3-((4-((3- aminopropyl)amino)butyl) amino)propyl)amino)-12- hydroxy-10,13- dimethylhexadecahydro-1H- cyclopenta[a]phenanthren-17- yl)pentanoyl)-D-valinate |
| 053 | 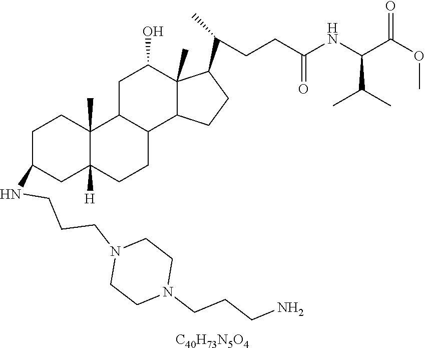 [SMILES: COC(=O)[C@H](NC(=O)CC[C@@H](C)[C@H]1CCC2C3CC[C@@H]4C[C@@H](NCCCN5CCN(CCCN)CC5)CC[C@]4(C)C3C[C@H](O)[C@@]21C)C(C)C ; "C40H73N5O4"] | methyl ((4R)-4- ((3S,5R,10S,12S,13R,17R)-3- ((3-(4-(3- aminopropyl)piperazin-1- yl)propyl)amino)-12-hydroxy- 10,13- dimethylhexadecahydro-1H- cyclopenta[a]phenanthren-17- yl)pentanoyl)-D-valinate |
| 054 | 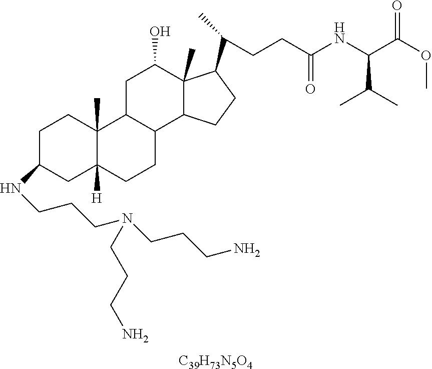 [SMILES: COC(=O)[C@H](NC(=O)CC[C@@H](C)[C@H]1CCC2C3CC[C@@H]4C[C@@H](NCCCN(CCCN)CCCN)CC[C@]4(C)C3C[C@H](O)[C@@]21C)C(C)C ; "C39H73N5O4"] | methyl ((4R)-4- ((3S,5R,10S,12S,13R,17R)-3- ((3-(bis(3- aminopropyl)amino)propyl) amino)-12-hydroxy-10,13- dimethylhexadecahydro-1H- cyclopenta[a]phenanthren-17- yl)pentanoyl)-D-valinate |
| 055 | 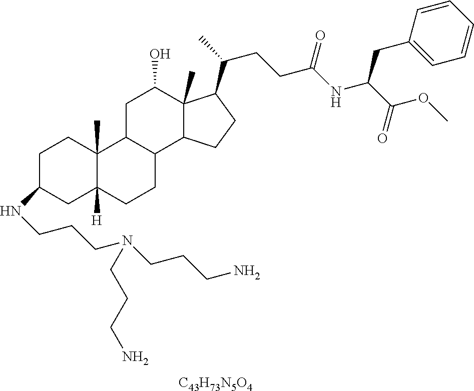 [SMILES: COC(=O)[C@H](Cc1ccccc1)NC(=O)CC[C@@H](C)[C@H]1CCC2C3CC[C@@H]4C[C@@H](NCCCN(CCCN)CCCN)CC[C@]4(C)C3C[C@H](O)[C@@]21C ; "C43H73N5O4"] | methyl ((4R)-4- ((3S,5R,10S,12S,13R,17R)-3- ((3-(bis(3- aminopropyl)amino)propyl) amino)-12-hydroxy-10,13- dimethylhexadecahydro-1H- cyclopenta[a]phenanthren-17- yl)pentanoyl)-L- phenylalaninate |
| 056 | 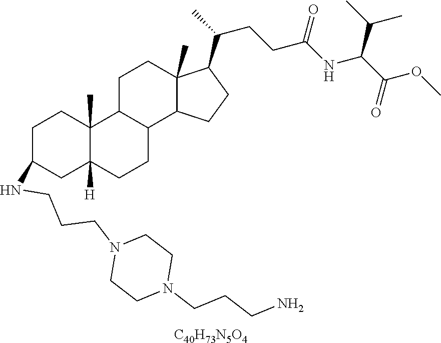 [SMILES: COC(=O)[C@@H](NC(=O)CC[C@@H](C)[C@H]1CCC2C3CC[C@@H]4C[C@@H](NCCCN5CCN(CCCN)CC5)CC[C@]4(C)C3CC[C@@]21C)C(C)C ; "C40H73N5O4"] | methyl ((4R)-4- ((3S,5R,10S,13R,17R)-3-((3- (4-(3-aminopropyl)piperazin- 1-yl)propyl)amino)-10,13- dimethylhexadecahydro-1H- cyclopenta[a]phenanthren-17- yl)pentanoyl)-L-valinate |
| 057 | 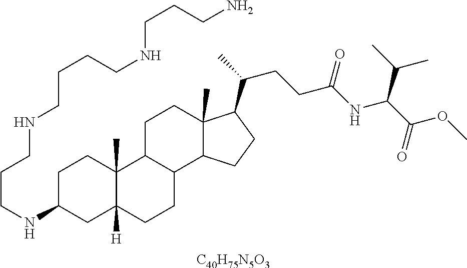 [SMILES: COC(=O)[C@@H](NC(=O)CC[C@@H](C)[C@H]1CCC2C3CC[C@@H]4C[C@@H](NCCCNCCCCNCCCN)CC[C@]4(C)C3CC[C@@]21C)C(C)C ; "C40H75N5O3"] | methyl ((4R)-4- ((3S,5R,10S,13R,17R)-3-((3- ((4-((3- aminopropyl)amino)butyl) amino)propyl)amino)-10,13- dimethylhexadecahydro-1H- cyclopenta[a]phenanthren-17- yl)pentanoyl)-L-valinate |
| 058 | 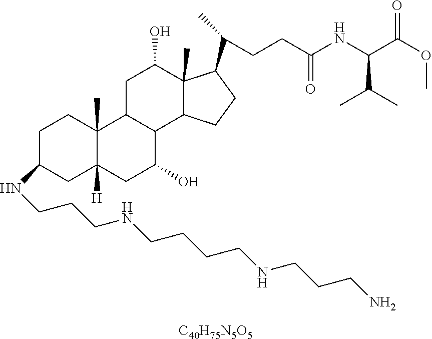 [SMILES: COC(=O)[C@H](NC(=O)CC[C@@H](C)[C@H]1CCC2C3C(C[C@H](O)[C@@]21C)[C@@]1(C)CC[C@H](NCCCNCCCCNCCCN)C[C@H]1C[C@H]3O)C(C)C ; "C40H75N5O5"] | methyl ((4R)-4- ((3S,5S,7R,10S,12S,13R,17R)- 3-((3-((4-((3- aminopropyl)amino)butyl) amino)propyl)amino)-7,12- dihydroxy-10,13- dimethylhexadecahydro-1H- cyclopenta[a]phenanthren-17- yl)pentanoyl)-D-valinate |
| 059 | 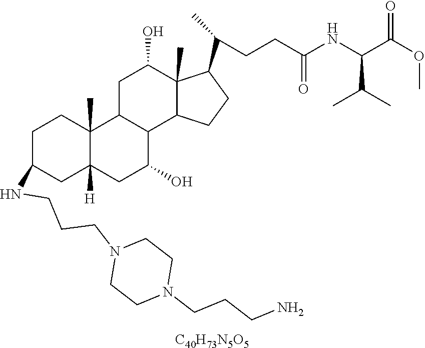 [SMILES: COC(=O)[C@H](NC(=O)CC[C@@H](C)[C@H]1CCC2C3C(C[C@H](O)[C@@]21C)[C@@]1(C)CC[C@H](NCCCN2CCN(CCCN)CC2)C[C@H]1C[C@H]3O)C(C)C ; "C40H73N5O5"] | methyl ((4R)-4- ((3S,5S,7R,10S,12S,13R,17R)- 3-((3-(4-(3- aminopropyl)piperazin-1- yl)propyl)amino)-7,12- dihydroxy-10,13- dimethylhexadecahydro-1H- cyclopenta[a]phenanthren-17- yl)pentanoyl)-D-valinate |
| 060 | 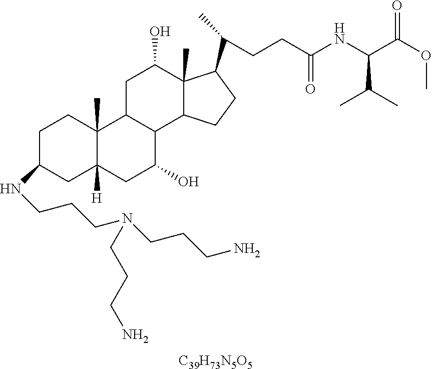 [SMILES: COC(=O)[C@H](NC(=O)CC[C@@H](C)[C@H]1CCC2C3C(C[C@H](O)[C@@]21C)[C@@]1(C)CC[C@H](NCCCN(CCCN)CCCN)C[C@H]1C[C@H]3O)C(C)C ; "C39H73N5O5"] | methyl ((4R)-4- ((3S,5S,7R,10S,12S,13R,17R)- 3-((3-(bis(3- aminopropyl)amino)propyl) amino)-7,12-dihydroxy-10,13- dimethylhexadecahydro-1H- cyclopenta[a]phenanthren-17- yl)pentanoyl)-D-valinate |
| 061 | 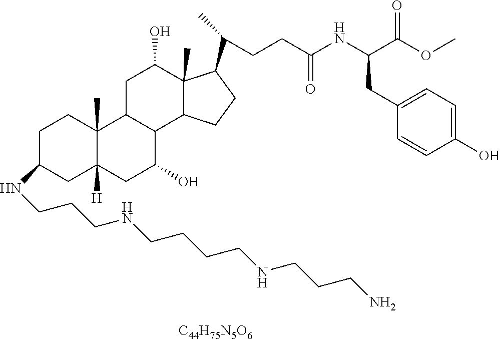 [SMILES: COC(=O)[C@@H](Cc1ccc(O)cc1)NC(=O)CC[C@@H](C)[C@H]1CCC2C3C(C[C@H](O)[C@@]21C)[C@@]1(C)CC[C@H](NCCCNCCCCNCCCN)C[C@H]1C[C@H]3O ; "C44H75N5O6"] | methyl ((4R)-4- ((3S,5S,7R,10S,12S,13R,17R)- 3-((3-((4-((3- aminopropyl)amino)butyl) amino)propyl)amino)-7,12- dihydroxy-10,13- dimethylhexadecahydro-1H- cyclopenta[a]phenanthren-17- yl)pentanoyl)-D-tyrosinate |

In one embodiment, the compound of formula (I) is selected from the compounds of Table 2 below, and pharmaceutically acceptable salts and/or solvates thereof.

TABLE 2

| Cpd | Name |
|---|---|
| 010 | methyl ((4R)-4-((3S,5S,7R,10S,13R,17R)-3-((2-((2-((2- |
| | aminoethyl)amino)ethyl)amino)ethyl)amino)-7-hydroxy-10,13- |
| | dimethylhexadecahydro-1H-cyclopenta[a]phenanthren-17-yl)pentanoyl)- |
| | D-phenylalaninate |
| 007 | methyl ((4R)-4-((3S,5S,7R,10S,13R,17R)-3-((3-((3- |
| | aminopropyl)amino)propyl)amino)-7-hydroxy-10,13- |
| | dimethylhexadecahydro-1H-cyclopenta[a]phenanthren-17-yl)pentanoyl)- |
| | D-valinate |
| 039 | methyl ((4R)-4-((3S,5S,7R,10S,13R,17R)-3-((3-((4-((3- |
| | aminopropyl)amino)butyl)amino)propyl)amino)-7-hydroxy-10,13- |
| | dimethylhexadecahydro-1H-cyclopenta[a]phenanthren-17-yl)pentanoyl)- |
| | L-tyrosinate |
| 046 | methyl ((4R)-4-((3S,5S,7R,10S,13R,17R)-3-((3-((2-((3- |
| | aminopropyl)amino)ethyl)amino)propyl)amino)-7-hydroxy-10,13- |
| | dimethylhexadecahydro-1H-cyclopenta[a]phenanthren-17-yl)pentanoyl)- |
| | D-prolinate |
| 060 | methyl ((4R)-4-((3S,5S,7R,10S,12S,13R,17R)-3-((3-(bis(3- |
| | aminopropyl)amino)propyl)amino)-7,12-dihydroxy-10,13- |
| | dimethylhexadecahydro-1H-cyclopenta[a]phenanthren-17-yl)pentanoyl)- |
| | D-valinate |

In one embodiment, the compound of formula (I) is selected from the compounds of Table 3 below.

TABLE 3

| Cpd | Name |
|---|---|
| 013 | methyl ((4R)-4-((3S,5S,7R,10S,13R,17R)-3-((3-((2-((3- |
| | aminopropyl)amino)ethyl)amino)propyl)amino)-7-hydroxy-10,13- |
| | dimethylhexadecahydro-1H-cyclopenta[a]phenanthren-17-yl)pentanoyl)- |
| | L-valinate |
| 014 | methyl ((4R)-4-((3S,5S,7R,10S,13R,17R)-3-((3-((4-((3- |
| | aminopropyl)amino)butyl)amino)propyl)amino)-7-hydroxy-10,13- |
| | dimethylhexadecahydro-1H-cyclopenta[a]phenanthren-17-yl)pentanoyl)- |
| | L-phenylalaninate |
| 044 | methyl (2S,3R)-2-((4R)-4-((3S,5S,7R,10S,13R,17R)-3-((3-((4-((3- |
| | aminopropyl)amino)butyl)amino)propyl)amino)-7-hydroxy-10,13- |
| | dimethylhexadecahydro-1H-cyclopenta[a]phenanthren-17- |
| | yl)pentanamido)-3-methylpentanoate |
| 007 | methyl ((4R)-4-((3S,5S,7R,10S,13R,17R)-3-((3-((3- |
| | aminopropyl)amino)propyl)amino)-7-hydroxy-10,13- |
| | dimethylhexadecahydro-1H-cyclopenta[a]phenanthren-17-yl)pentanoyl)- |
| | D-valinate |
| 052 | methyl ((4R)-4-((3S,5R,10S,12S,13R,17R)-3-((3-((4-((3- |
| | aminopropyl)amino)butyl)amino)propyl)amino)-12-hydroxy-10,13- |
| | dimethylhexadecahydro-1H-cyclopenta[a]phenanthren-17-yl)pentanoyl)- |
| | D-valinate |

According to one embodiment, the compound is selected from the compounds of Table 2 and/or Table 3 herein.

All references herein to a compound of the invention (e.g., “compound of formula (I)”) include references to salts, solvates, multi component complexes and liquid crystals thereof. All references herein to a compound of the invention include references to polymorphs and crystal habits thereof. All references herein to a compound of the invention include references to isotopically-labelled compounds thereof, including deuterated compounds thereof. All references herein to a compound of the invention include references to stereoisomers thereof. All herein references to a compound of the invention include references to pharmaceutically acceptable prodrugs and predrugs thereof.

In particular, the compounds of the invention (e.g., a “compound of formula (I)”) may be in the form of pharmaceutically acceptable salts. According to one embodiment, the compound of the invention is a pharmaceutically acceptable salt. Pharmaceutically acceptable salts include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts. Suitable base salts are formed from bases which form non-toxic salts.

Examples include the aluminum, arginine, benzathine, calcium, choline, diethylamine, 2-(diethylamino)ethanol, diolamine, ethanolamine, glycine, 4-(2-hydroxyethyl)-morpholine, lysine, magnesium, meglumine, morpholine, olamine, potassium, sodium, tromethamine and zinc salts. Hemisalts of acids and bases may also be formed, for example, hemisulphate and hemicalcium salts. When a compound contains an acidic group as well as a basic group the compound may also form internal salts, and such compounds are within the scope of the invention. When a compound contains a hydrogen-donating heteroatom (e.g., NH), the invention also encompasses salts and/or isomers formed by transfer of said hydrogen atom to a basic group or atom within the molecule. Pharmaceutically acceptable salts of compounds of the invention may be prepared by one or more of these methods: (i) by reacting the compound with the desired acid; (ii) by reacting the compound with the desired base; (iii) by removing an acid- or base-labile protecting group from a suitable precursor of the compound or by ring-opening a suitable cyclic precursor, e.g., a lactone or lactam, using the desired acid; and/or (iv) by converting one salt of the compound to another by reaction with an appropriate acid or by means of a suitable ion exchange column. All these reactions are typically carried out in solution. The salt may precipitate from solution and be collected by filtration or may be recovered by evaporation of the solvent. The degree of ionization in the salt may vary from completely ionized to almost non-ionized.

According to one embodiment, the salt of the compound of formula (I) is selected from a citric acid salt (citrate), a hydrochloric acid (HCl) salt (chloride) and a lactic acid salt (lactate). In one embodiment, the salt of the compound of formula (I) is a citric acid salt. In one embodiment, the salt of the compound of formula (I) is a hydrochloric acid (HCl) salt. In one embodiment, the salt of the compound of formula (I) is a lactic acid salt.

In particular, the compounds of the invention (e.g., a “compound of formula (I)”) may be in the form of pharmaceutically acceptable solvates. According to one embodiment, the compound of the invention is a pharmaceutically acceptable solvate.

According to one embodiment, the compound of the invention is a pharmaceutically acceptable salt and solvate.

In particular, the compounds of the invention (e.g., a “compound of formula (I)”) include at least one asymmetric center(s) and thus may exist as different stereoisomeric forms. Accordingly, all references to a compound of the invention include all possible stereoisomers and includes not only the racemic compounds, but the individual enantiomers and their non-racemic mixtures as well. Non-racemic mixtures may comprise any amounts of each distinct stereoisomer, for example one stereoisomer may be preponderant (e.g., a 90/10 or 80/20 mixture), or the enantiomeric ratio may be close to a racemic mixture (e.g., a 40/60 mixture). When a compound is desired as a single enantiomer, such single enantiomer may be obtained by stereospecific synthesis, by resolution of the final product or any convenient intermediate, or by chiral chromatographic methods as each are known in the art. Resolution of the final product, an intermediate, or a starting material may be carried out by any suitable method known in the art. Diastereoisomeric ratios may be determined by methods known in the art such as, for example, high-performance liquid chromatography (HPLC). Enantiomeric ratios may be determined by methods known in the art such as, for example, chiral HPLC (i.e., HPLC wherein the stationary phase is a chiral column).

According to one embodiment, the compound of the invention is a mixture of two diastereomers (beta/alpha) and the beta isomer is present in the mixture in an amount equal to or higher than about 90%. In one embodiment, the beta isomer is present in the mixture in an amount equal to or higher than about 91%, about 92%, about 93%, about 94%, about 95%, about 96%, about 97%, about 98% or about 99%. In one embodiment, the beta isomer is present in the mixture in an amount equal to or higher than about 94%.

In one embodiment, the beta isomer is present in the mixture in an amount equal to or higher than about 95%. In one embodiment, the compound of the invention substantially consists of the beta isomer.

According to another embodiment, the compound of the invention is a mixture of two diastereomers (beta/alpha) and the beta isomer is present in the mixture in an amount equal to or lower than about 75%, about 80% or about 85%. In one embodiment, the beta isomer is present in the mixture in an amount equal to or lower than about 80%.

In one embodiment, the compound of the invention is selected from the pharmaceutically acceptable salts and/or stereoisomers of compounds of formula (I) as listed on Table 4 below, and solvates thereof.

TABLE 4

| | | | Stereochemistry |
|---|---|---|---|
| Final | Base | | (Diastereoisomers |
| Cpd | Cpd | Salt | beta:alpha) |
| S001 | 001 | HCl salt | 95:05 |
| S002 | 002 | HCl salt | 95:05 |
| S003 | 003 | HCl salt | 95:05 |
| S004 | 004 | HCl salt | 95:05 |
| S005 | 005 | HCl salt | 95:05 |
| S006 | 006 | HCl salt | 95:05 |
| S007 | 007 | HCl salt | 96:04 |
| S008 | 008 | HCl salt | 95:05 |
| S009 | 009 | HCl salt | 95:05 |
| S010 | 010 | HCl salt | 95:05 |
| S011 | 011 | HCl salt | 95:05 |
| S012 | 012 | HCl salt | 95:05 |
| S013 | 013 | HCl salt | 95:05 |
| S014 | 014 | HCl salt | 95:05 |
| S015 | 015 | HCl salt | 95:05 |
| S016 | 016 | HCl salt | 95:05 |
| S017 | 007 | HCl salt | 80:20 |
| S018 | 018 | HCl salt | 95:05 |
| S019 | 007 | HCl salt | 94:06 |
| S020 | 020 | HCl salt | 95:05 |
| S021 | 007 | HCl salt | 90:10 |
| S022 | 022 | HCl salt | 95:05 |
| S023 | 023 | HCl salt | 95:05 |
| S024 | 024 | HCl salt | 95:05 |
| S025 | 025 | HCl salt | 95:05 |
| S026 | 026 | HCl salt | 95:05 |
| S027 | 004 | HCl salt | 80:20 |
| S028 | 028 | HCl salt | 95:05 |
| S029 | 029 | HCl salt | 95:05 |
| S030 | 030 | HCl salt | 95:05 |
| S031 | 031 | HCl salt | 95:05 |
| S032 | 032 | HCl salt | 95:05 |
| S034 | 034 | HCl salt | 95:05 |
| S035 | 035 | HCl salt | 95:05 |
| S036 | 036 | HCl salt | 95:05 |
| S037 | 037 | HCl salt | 95:05 |
| S038 | 038 | HCl salt | 95:05 |
| S039 | 039 | HCl salt | 95:05 |
| S040 | 040 | HCL salt | 90:10 |
| S041 | 041 | HCl salt | 95:05 |
| S042 | 042 | HCl salt | 95:05 |
| S043 | 043 | HCl salt | 95:05 |
| S044 | 044 | HCl salt | 95:05 |
| S045 | 045 | HCl salt | 95:05 |
| S046 | 046 | HCl salt | 95:05 |
| S047 | 007 | Lactic acid salt | 95:05 |
| S048 | 007 | Citric acid salt | 95:05 |
| S049 | 049 | HCl salt | 95:05 |
| S050 | 050 | HCl salt | 95:05 |
| S051 | 051 | HCl salt | 95:05 |
| S052 | 052 | HCl salt | 95:05 |
| S053 | 053 | HCl salt | 95:05 |
| S054 | 054 | HCl salt | 95:05 |
| S055 | 055 | HCl salt | 95:05 |
| S056 | 056 | HCl salt | 95:05 |
| S057 | 057 | HCl salt | 95:05 |
| S058 | 058 | HCl salt | 95:05 |
| S059 | 059 | HCl salt | 95:05 |
| S060 | 060 | HCl salt | 95:05 |
| S061 | 061 | HCl salt | 95:05 |

The compounds of Table 4 may, for example, be prepared as described in the experimental part (Example 1 below).

In one embodiment, the compound of the invention is selected from the pharmaceutically acceptable salts and/or stereoisomers of compounds of formula (I) as listed on Table 5 below, and solvates thereof.

TABLE 5

| | | | Stereochemistry |
|---|---|---|---|
| Final | Base | | (Diastereoisomers |
| Cpd | Cpd | Salt | beta:alpha) |
| S010 | 010 | HCl salt | 95:05 |
| S019 | 007 | HCl salt | 94:06 |
| S039 | 039 | HCl salt | 95:05 |
| S046 | 046 | HCl salt | 95:05 |
| S060 | 060 | HCl salt | 95:05 |

In one embodiment, the compound of the invention is selected from the pharmaceutically acceptable salts and/or stereoisomers of compounds of formula (I) as listed on Table 6 below, and solvates thereof.

TABLE 6

| | | | Stereochemistry |
|---|---|---|---|
| Final | Base | | (Diastereoisomers |
| Cpd | Cpd | Salt | beta:alpha) |
| S013 | 013 | HCl salt | 95:05 |
| S014 | 014 | HCl salt | 95:05 |
| S044 | 044 | HCl salt | 95:05 |
| S047 | 007 | Lactic acid salt | 95:05 |
| S048 | 007 | Citric acid salt | 95:05 |
| S052 | 052 | HCl salt | 95:05 |

According to one embodiment, the compound is selected from the compounds of Table 5 and/or Table 6 herein.

### Pharmaceutical Composition

Another object of the present invention is a composition comprising a compound according to the invention, as described herein. In one embodiment, said composition is a pharmaceutical composition and further comprises at least one pharmaceutically acceptable carrier. Consequently, another object of the present invention is a pharmaceutical composition comprising a compound according to the invention, as described herein, and at least one pharmaceutically acceptable carrier.

According to a first embodiment, the pharmaceutical composition comprises the compound according to the invention as sole therapeutic agent. In one embodiment, the pharmaceutical composition does not comprise any other anti-infective agent. In one embodiment, the pharmaceutical composition does not comprise any other antibacterial agent. According to a second embodiment, the pharmaceutical composition further comprises at least another therapeutic agent. In one embodiment, the pharmaceutical composition further comprises at least another anti-infective agent. In one embodiment, the other anti-infective agent is selected from cyclins, macrolides, phenicols and beta-lactams.

Another object of the present invention is a medicament comprising a compound according to the invention, as described herein.

### Kit

Another object of the invention is a kit comprising a compound according to the invention, as described herein, and instructions for use.

The present invention also relates to a kit comprising:
a pharmaceutical composition comprising the compound according to the invention and
another separate pharmaceutical composition comprising at least another therapeutic agent such as, for example, an anti-infective agent.

### Medical Use of the Compound

The present invention also relates to a compound, a composition, or a pharmaceutical composition according to the invention, as described herein, for use as a medicament.

The present invention also relates to a compound, a composition, or a pharmaceutical composition according to the invention, as described herein, for use in the treatment of an infectious disease.

The present invention also relates to a compound, a composition, or a pharmaceutical composition according to the invention, as described herein, for use as anti-persisters agent in the treatment of an infectious disease.

The present invention also relates to a compound, a composition, or a pharmaceutical composition according to the invention, as described herein, for use in the treatment of a chronic infection, a relapsing infection, a recalcitrant infection, a persistent infection, a persister related-infection, a persister related-chronic infection, a persister related-relapsing infection, a persister related-recalcitrant infection, or a persister related-persistent infection. The present invention also relates to a compound, a composition, or a pharmaceutical composition according to the invention, as described herein, for use for killing or inhibiting the growth of persister cells. The present invention also relates to a compound, a composition, or a pharmaceutical composition according to the invention, as described herein, for use for treating a microbial infection comprising at least one persister cell.

The present invention further relates to a method for treating an infectious disease in a subject in need thereof, comprising administering to the subject a compound, a composition, or a pharmaceutical composition according to the invention, as described herein. The present invention further relates to a method for treating a chronic infection, a relapsing infection, a recalcitrant infection, a persistent infection, a persister related-infection, a persister related-chronic infection, a persister related-relapsing infection, a persister related-recalcitrant infection, or a persister related-persistent infection in a subject in need thereof, comprising administering to the subject a compound, a composition, or a pharmaceutical composition according to the invention, as described herein. The present invention further relates to a method for killing or inhibiting the growth of persister cells in a subject in need thereof, comprising administering to the subject a compound, a composition, or a pharmaceutical composition according to the invention, as described herein. The present invention further relates to a method for treating a microbial infection comprising at least one persister cell in a subject in need thereof, comprising administering to the subject a compound, a composition, or a pharmaceutical composition according to the invention, as described herein.

The present invention further relates to the use of a compound, a composition, or a pharmaceutical composition according to the invention, as described herein, for the manufacture of a medicament for the treatment of an infectious disease in a subject in need thereof. The present invention further relates to the use of a compound, a composition, or a pharmaceutical composition according to the invention, as described herein, for the manufacture of a medicament for the treatment of a chronic infection, a relapsing infection, a recalcitrant infection, a persistent infection, a persister related-infection, a persister related-chronic infection, a persister related-relapsing infection, a persister related-recalcitrant infection, or a persister related-persistent infection in a subject in need thereof. The present invention further relates to the use of a compound, a composition, or a pharmaceutical composition according to the invention, as described herein, for the manufacture of a medicament for killing or inhibiting the growth of persister cells in a subject in need thereof. The present invention further relates to the use of a compound, a composition, or a pharmaceutical composition according to the invention, as described herein, for the manufacture of a medicament for the treatment of a microbial infection comprising at least one persister cell in a subject in need thereof.

The present invention also relates to the use of a compound, a composition, or a pharmaceutical composition according to the invention, as described herein, for treating an infectious disease in a subject in need thereof. The present invention also relates to the use of a compound, a composition, or a pharmaceutical composition according to the invention, as described herein, for treating a chronic infection, a relapsing infection, a recalcitrant infection, a persistent infection, a persister related-infection, a persister related-chronic infection, a persister related-relapsing infection, a persister related-recalcitrant infection, or a persister related-persistent infection in a subject in need thereof. The present invention also relates to the use of a compound, a composition, or a pharmaceutical composition according to the invention, as described herein, for killing or inhibiting the growth of persister cells in a subject in need thereof. The present invention also relates to the use of a compound, a composition, or a pharmaceutical composition according to the invention, as described herein, for treating a microbial infection comprising at least one persister cell in a subject in need thereof.

According to one embodiment, the compound is selected from the compounds of Table 2 and/or Table 3 herein. In one embodiment, the compound is selected from the compounds of Table 2 herein. In one embodiment, the compound is selected from the compounds of Table 3 herein. According to one embodiment, the compound is selected from the compounds of Table 4 and/or Table 5 herein. In one embodiment, the compound is selected from the compounds of Table 4 herein. In one embodiment, the compound is selected from the compounds of Table 5 herein.

According to one embodiment, the infectious disease is a bacterial disease, a viral disease, a fungal disease or a parasitic disease. In one embodiment, the infectious disease is a bacterial disease. In one embodiment, the infectious disease is a viral disease. In one embodiment, the infectious disease is a fungal disease. In one embodiment, the infectious disease is a parasitic disease. In one embodiment, the infectious disease is a bacterial disease or a fungal disease. In one embodiment, the bacterial or fungal disease is selected from cystic fibrosis, urinary tract infection and chronic otitis.

In one embodiment, the bacterial disease is caused by Gram-positive bacteria. In one embodiment, the bacterial disease is caused by Gram-negative bacteria. As used herein, “Gram-positive bacteria” refers to bacteria that retain the color of the crystal violet stain used in the Gram staining method of bacterial differentiation. Gram-positive bacteria are characterized by a bacterial cell wall composed of a thick layer of peptidoglycan. As opposed to “Gram-negative bacteria” which refers to bacteria that do not retain the crystal violet stain used in the Gram staining. Gram-negative bacteria are characterized by a bacterial cell wall composed of a thin layer of peptidoglycan in between an inner cytoplasmic cell membrane and a bacterial outer membrane. In one embodiment, the Gram-positive bacteria is selected from Staphylococcus bacteria, Enterococcus bacteria and Mycobacterium bacteria. In one embodiment, the Staphylococcus bacteria is Staphylococcus epidermidis or Staphylococcus aureus. In one embodiment, the Enterococcus bacteria is Enterococcus faecium. In one embodiment, the Mycobacterium bacteria is Mycobacterium tuberculosis. In one embodiment, the Gram-negative bacteria is selected from Pseudomonas bacteria, Escherichia bacteria, Klebsiella bacteria, Acinetobacter bacteria, Enterobacter bacteria or Legionella bacteria. In one embodiment, the Pseudomonas bacteria is Pseudomonas aeruginosa. In one embodiment, the Escherichia bacteria is Escherichia coli. In one embodiment, the Klebsiella bacteria is Klebsiella pneumoniae. In one embodiment, the Acinetobacter bacteria is Acinetobacter baumannii.

According to one embodiment, the subject is human. According to one embodiment, the subject is non-human. In one embodiment, the subject is selected from cattle, sheep, goats, pigs and poultry. In one embodiment, the subject is a meat-producing animal or a milk-producing animal.

### Administration Route and Dosage

For use in administration to a subject, the medicament, composition, or pharmaceutical composition according to the invention, as described herein, will be formulated. In one embodiment, the medicament, composition, or pharmaceutical composition is administered parenterally, orally, by inhalation, spray, rectally, nasally, or via an implanted reservoir. In one embodiment, the medicament, composition, or pharmaceutical composition is administered by injection, including, without limitation, subcutaneous, intravenous, intramuscular, intra-articular, intra-synovial, intra-sternal, intrathecal, intrahepatic, intralesional and intracranial injection or infusion techniques. Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

In one embodiment, the medicament, composition, or pharmaceutical composition is to be administered to the subject in need thereof in a therapeutically effective amount.

It will be however understood that the total daily usage of the compound, composition, pharmaceutical composition or medicament according to the present invention will be decided by the attending physician within the scope of sound medical judgment. The specific therapeutically effective dose level for any particular patient will depend upon a variety of factors including the disease being treated and the severity of the disease; activity of the compound employed; the age, body weight, general health, sex and diet of the subject; the time of administration, route of administration, and rate of excretion of the specific therapeutic agent employed; the duration of the treatment; drugs used in combination or coincidental with the specific therapeutic agent employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

In one embodiment, the dosage of the compound will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be about 0.05 to 0.5, about 0.5 to 5 or about 5 to 50 mg/kg per day. For oral administration, the compositions are preferably provided in the form of tablets containing from about 1.0 to 1000 milligrams of the active ingredient, particularly about 1.0, about 5.0, about 10.0, about 15.0, about 20.0, about 25.0, about 50.0, about 75.0, about 100.0, about 150.0, about 200.0, about 250.0, about 300.0, about 400.0, about 500.0, about 600.0, about 750.0, about 800.0, about 900.0, and about 1000.0 milligrams of the active ingredient for the symptomatic adjustment of the dosage to the patient to be treated. The compounds may be administered on a regimen of 1 to 4 times per day, preferably once or twice per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Co-Administration

According to a first embodiment, the medicament, composition, or pharmaceutical composition according to the invention, as described herein, is to be administered as sole therapeutic agent. In one embodiment, the medicament, composition, or pharmaceutical composition is not to be administered in combination with any other anti-infective agent.

According to a second embodiment, the medicament, composition, or pharmaceutical composition according to the invention, as described herein, is to be administered before, concomitantly with or after at least another therapeutic agent, such as, for example, an anti-infective or antiproliferative agent.

It will be understood by the one skilled in the art that the co-administration of the compound, composition, pharmaceutical composition or medicament according to the present invention with a particular therapeutic agent, which may be chosen among those recited herein but without being limited thereto, will depend on the disease or condition to be prevented and/or treated. Some examples of anti-infective agent suitable for co-administration according to the present invention include, without limitation, cyclines, macrolides, phenicols, beta-lactams.

### Non-Medical Use

The present invention also relates to the non-therapeutic use of a compound according to the invention, as described herein, as anti-infective agent. The present invention also relates to the non-therapeutic use of a compound according to the invention, as described herein, as anti-persisters agent.

According to one embodiment, the compound is selected from the compounds of Table 2 and/or Table 3 herein. In one embodiment, the compound is selected from the compounds of Table 2 herein. In one embodiment, the compound is selected from the compounds of Table 3 herein.

According to one embodiment, the use of the compound is for the disinfection of a surface. In this embodiment, “disinfection of a surface” refers to the case wherein the surface is not part of a human or animal body. For example, human or animal skin is not considered a “surface” in the sense of this embodiment. In one embodiment, the surface is comprised in a medical device, a cloth, a vehicle or a building. In one embodiment, the surface is comprised in a medical device such as, for example, a protheses, a bandage or a hospital bed. In one embodiment, the surface is comprised in a cloth, such as, for example, a medical blouse. In one embodiment, the surface is comprised in a vehicle, such as, for example, an ambulance. In one embodiment, the surface is comprised in a building, such as, for example, in a floor, a wall, a window or a pipe.

According to one embodiment, the use of the compound is for the purification of a liquid. In this embodiment, “purification of a liquid” refers to the case wherein the liquid is not part of a human or animal body. For example, human or animal blood is not considered a “liquid” in the sense of this embodiment. In one embodiment, the liquid is comprised in a container, a furniture or a building. In one embodiment, the liquid is comprised in a container, such as, for example, a bottle or a bag. In one embodiment, the liquid is comprised in a furniture or a building, such as, for example, a pool, a reservoir or a tank.

According to one embodiment, the use of the compound is for eliminating any one of the bacteria as described hereinabove, or any one of their combinations. In this embodiment, “eliminating bacteria” refers to the case wherein the bacteria are not in a human or animal body, nor in contact with a human or animal body.

The present invention also relates to a method for the disinfection of a surface and/or the purification of a liquid; wherein “disinfection of a surface” and “purification of a liquid” have the meaning as defined hereinabove.

The present invention also relates to a method for eliminating any one of the bacteria as described hereinabove; wherein “eliminating bacteria” has the meaning as defined hereinabove.

### Manufacturing Process

The compound of the invention as described herein may be manufactured by synthetic methods well-known in the art.

This invention also relates to a process for manufacturing a compound of the invention as described herein.

According to one embodiment, the process comprises:
(a) a step of reacting the carboxylic acid function in position 20 of a bile acid with the secondary amine function of an amino acid, thereby obtaining an amide;
(b) a step of oxidizing the hydroxyl (OH) in position 3 of the bile acid intermediate obtained in step (a), thereby obtaining a ketone;
(c) a step of reacting the ketone of the bile acid intermediate obtained in step (b) with a primary amine, thereby obtaining an imine; and
(d) a step of reduction of the imine obtained in step (c), thereby obtaining the compound of the invention.

This embodiment in illustrated on Scheme 1 below.

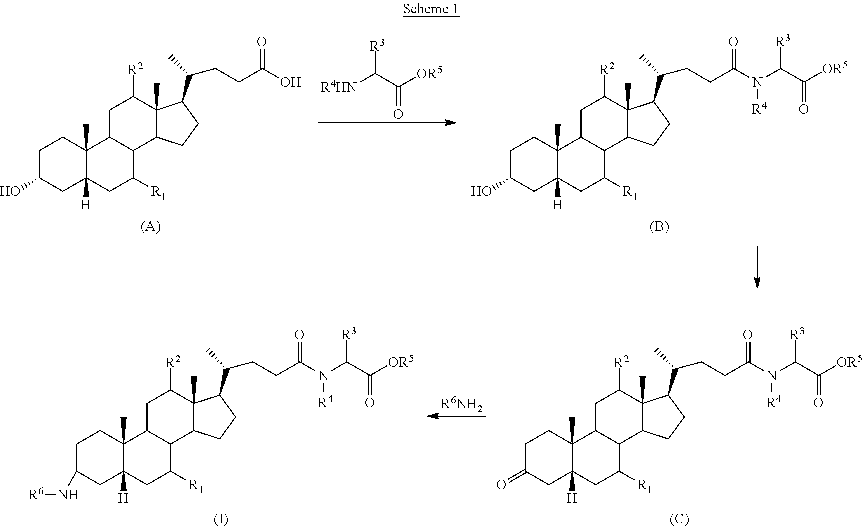

Non-limiting examples of suitable bile acids are represented hereinabove under formula (I). According to one embodiment, the bile acid is selected from deoxycholic acid, cholic acid, chenodeoxycholic acid, ursodeoxycholic acid and lithocholic acid. In one embodiment, the bile acid is selected from deoxycholic acid, cholic acid, chenodeoxycholic acid and lithocholic acid. In the process of the invention, the amino acid may be natural or non-natural. According to one embodiment, the amino acid is natural.

According to one embodiment, the step (c) of reacting the ketone of the bile acid and the step (d) of reduction of the imine obtained in step (c) are carried out in the same reaction medium (in situ), i.e., both reactions are carried out without any intermediate purification and/or separation step for the imine.

The process according to the invention may further comprise purification and/or separation steps well-known in the art.

The process according to the invention is advantageous over state-of-the art process for manufacturing squalamine, as it comprises only 3 or 4 steps, whereas squalamine preparation requires more than 13 steps.

### Examples

The present invention is further illustrated by the following examples.

### Example 1: Synthesis of the compounds

### Materials and Methods

All the syntheses were carried out with solvents purified according to the usual methods. Commercial reagents obtained from TCI Europe and Sigma-Aldrich were used directly without prior purification. The progress of the reactions was monitored by thin layer chromatography (TLC) using a 10% solution of phosphomolybdic acid in absolute ethanol as a visualizing agent. Reactions under microwave irradiation were performed using the Biotage Initiator+apparatus. The following abbreviations were used to name the solvents: PET: Petroleum ether, EtOAc: Ethyl acetate, CH₂Cl₂: Dichloromethane, MeOH: Methanol and NH₄·OH: 32% by weight aqueous ammonia solution. The chemical structures of the products obtained were verified by proton (¹H) and carbon (¹³C) NMR (Nuclear Magnetic Resonance) analysis performed on a Bruker Avance NEO 400 MHz NanoBay NMR spectrometer. Chemical shifts (d) were expressed in parts per million (ppm). The residual solvent peak was used as reference with the following values: deuterated chloroform (CDCl₃) d ¹H=7.26 ppm, d ¹³C=77.16 ppm; deuterated methanol (CD₃OD) d ¹H=4.87 ppm, d ¹³C=49.00 ppm. [410] The following abbreviations were used to write the ¹H spectrum: s=singlet, d=doublet, t=triplet, q=quadruplet and m=multiplet. The coupling constants (J), expressed in Hz, were determined for doublets, triplets and quadruplets. Mass spectrometry analysis of the final products (sonamines) was performed using a SYNAPT G2 HDMS (Waters), after electrospray ionization (ESI-MS). The sample was ionized in positive-mode electrospray under the following conditions: electrospray voltage: 2.8 kV; orifice voltage: 20 V; nebulizer gas flow rate (nitrogen): 100 L/h.

The diastereomeric ratios were determined by high-performance liquid chromatography (HPLC) using an Agilent 1100 series apparatus and a brand new Ascentis® HPLC column (Express C18, 4.6 mm×100 mm, 2.7 μm), with a water/methanol gradient according to the following procedure (A: water+0.1% of trifluoroacetic acid (TFA), B: Methanol+0.1% of TFA):

| Time (Min) | A [%] | B [%] | Flow (mL/min) |
|---|---|---|---|
| 0 | 90 | 10 | 0.1 |
| 0.01 | 90 | 10 | 1.0 |
| 4.00 | 10 | 90 | 1.0 |
| 7.00 | 10 | 90 | 1.0 |
| 9.00 | 90 | 10 | 1.0 |
| 10.00 | 90 | 10 | 1.0 |

A. Peptide coupling procedure—Synthesis of alcohols

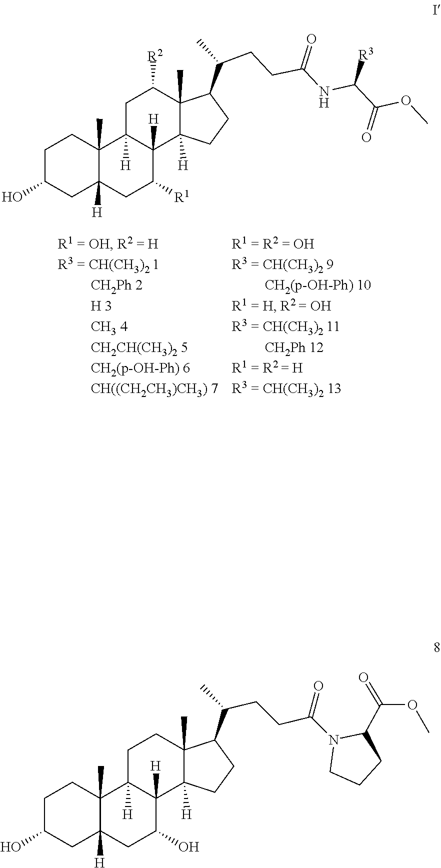

All alcohols I′ presented in this section were synthesized from bile acid and the methyl ester of the amino acid under consideration, following the same procedure detailed for the synthesis of methyl-L-valinate chenodeoxycholate 1.

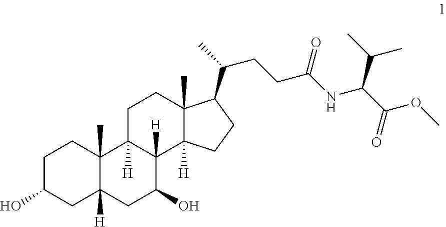

Methyl-L-valinate chenodeoxycholate 1. In a flask fitted with a magnetic stirring bar, 2 g of chenodeoxycholic acid (5.095 mmol), 0.856 g of L-valine methyl ester (5.095 mmol) and 2,25 g benzotriazol-1-yloxtris(dimethylamino) phosphonium hexafluorophosphate (5.095 mmol) are dissolved in 40 mL CH₂C₂ and 3.5 mL of diisopropylethylamine (20.38 mmol) is added. After 12 h stirring at 20° C., 20 mL of a saturated ammonium chloride solution is added and the medium is stirred for 2 h. The two phases are separated, the organic phase is washed twice with 20 mL of a saturated ammonium chloride solution and the aqueous phase is extracted with 20 mL of CH₂C₂.

The combined organic phases are washed twice with 20 mL of a saturated solution of sodium chloride. The organic phases are dried over anhydrous sodium sulphate and filtered. The crude product thus obtained is purified by chromatography on silica gel (eluent: PET then PET/EtOAc (1/1) then EtOAc). Methyl-L-valinate chenodeoxycholate 1 was obtained as a white solid in >95% yield. Methyl-L-valinate chenodeoxycholate 1 (C₃₀H₅₁NO₅). NMR ¹H (250 MHz, CDCl₃): δ (ppm)=5.97 (d, J=5.94 Hz, 1H), 4.58 (m, 1H), 4.15 (q, J₁=4.15 Hz, J₂=4.12 Hz 1H), 3.85-3.78 (m, 2H), 3.51 (m, 1H), 2.36-2.05 (m, 7H), 2.00-1.02 (m, 35H), 0.64 (s, 3H). NMR ¹³C (63 MHz, CDCl₃): δ (ppm)=173.61, 172.93, 72.12, 68.62, 60.53, 56.94, 55.96, 52.26, 50.54, 42.80, 41.57, 39.90, 39.52, 35.56, 35.14, 34.67, 33.58, 32.92, 31.78, 31.44, 30.74, 28.31, 23.80, 22.88, 20.68, 19.05, 18.47, 17.97, 14.31, 11.88.

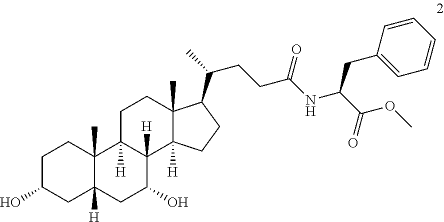

Methyl-L-phenylalaninate chenodeoxycholate 2 (C₃₄H₅₁NO₅). NMR ¹H (250 MHz, CD₃OD): δ (ppm)=7.60-7.28 (m, 5H), 5.56 (t, J=5.58 Hz, 1H), 4.27-4.08 (m, 1H), 3.87 (s, 1H), 3.77 (s, 3H), 3.51-3.41 (m, 1H), 2.48-2.22 (m, 3H), 2.10-0.94 (m, 33H), 0.74 (s, 3H). NMR ¹³C (63 MHz, CD₃OD): δ (ppm)=176.31, 172.67, 152.15, 137.55, 129.86, 128.77, 72.81, 68.97, 61.49, 58.12, 57.32, 52.94, 51.49, 43.65, 43.15, 41.02, 40,75, 40,44, 36,76, 36,56, 36,19, 35,90, 34,02, 33,48, 33,03, 31,35, 29,20, 24,61, 23,43, 21,78, 20,87, 18,93, 14,47, 12,23. Efficiency: >95%.

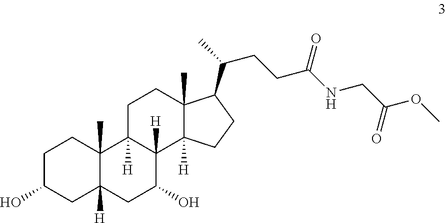

Methyl-L-glycinate chenodeoxycholate 3 (C₂₇H₄₅NO₅). NMR ¹H (250 MHz, CD₃OD): δ (ppm)=4.06 (s, 1H), 3.98-3.88 (m, 1H), 3.39-2.96 (m, 3H), 2.88-2.63 (m, 1H), 2.48-1.54 (m, 22H), 1.41-0.99 (m, 14H), 0.80 (s, 3H). NMR ¹³C (63 MHz, CD₃OD): δ (ppm)=177.10, 171.83, 72.78, 68.94, 57.31, 55.82, 52.53, 51.48, 43.76, 43.63, 43.10, 41.80, 40.72, 40.41, 36.52, 36.16, 35.86, 33.70, 33.02, 31.32, 29.18, 24.58, 23.40, 21.75, 18.90, 13.08, 12.21. Yield: >95%.

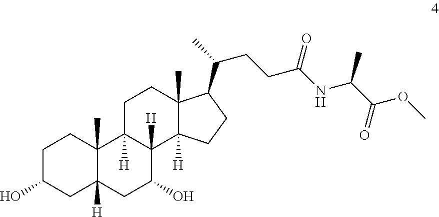

Methyl-L-alaninate chenodeoxycholate 4 (C₂₈H₄₇NO₅). NMR ¹H (250 MHz, CD₃OD): δ (ppm)=4.57 (m, 1H), 4.04-3.83 (m, 2H), 3.56-3.43 (m, 2H), 3.37-3.26 (m, 1H), 2.54-1.53 (m, 21H), 1.43-0.90 (m, 17H), 0.79 (s, 3H). NMR ¹³C (63 MHz, CD₃OD): δ (ppm)=176.40, 174.73, 72.76, 68.91, 57.31, 55.80, 52.65, 51.46, 49.32, 43.61, 43.08, 41.00, 40.70, 40.38, 36.74, 36.52, 35.85, 33.62, 33.02, 31.31, 29.18, 24.56, 23.41, 21.74, 18.92, 17.34, 13.07, 12.22. Yield: >95%.

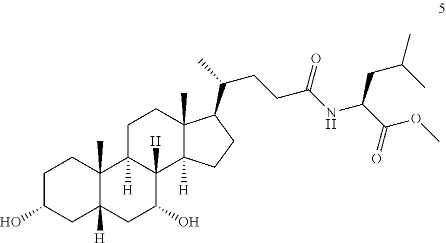

Methyl-L-leucinate chenodeoxycholate 5 (C₃₁H₅₃NO₅). NMR ¹H (400 MHz, CD₃OD): δ (ppm)=4.38 (d, J=4.38 Hz, 1H), 3.81 (s, 1H), 3.72 (s, 1H), 2.37-2.17 (m, 3H), 2.03-1.46 (m, 17H), 1.39-1.09 (m, 14H), 1.01-0.92 (m, 13H), 0.71 (s, 3H). NMR ¹³C (101 MHz, CD₃OD): δ (ppm)=176.71, 173.68, 72.71, 68.87, 58.11, 57.26, 55.71, 52.36, 51.41, 43.57, 43.01, 40.95, 40.63, 40.30, 38.07, 36.76, 36.48, 36.09, 35.81, 33.91, 33.57, 33.16, 29.19, 26.30, 24.54, 23.42, 21.72, 18.91, 15.98, 12.23, 11.64. Yield: >95%.

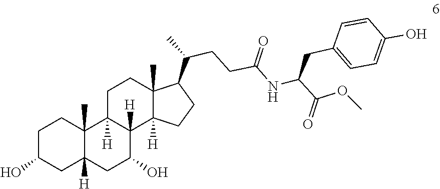

Methyl-L-tyrosinate chenodeoxycholate 6 (C₃₄H₅₁NO₆). NMR ¹H (250 MHz, CD₃OD): δ (ppm)=7.06 (m, 2H), 6.76 (m, 2H), 4.62 (m, 1H), 3.90-3.74 (m, 2H), 3.69-3.42 (m, 1H), 3.34-2.84 (m, 5H), 2.39-1.46 (m, 20H), 1.36-0.87 (m, 15H), 0.73 (s, 3H). NMR ¹³C (63 MHz, CD₃OD): δ (ppm)=176.56, 173.74, 157.31, 131.13, 128.76, 116.21, 72.79, 68.98, 57.28, 55.76, 55.34, 52.60, 51.45, 43.60, 43.09, 40.99, 40,69, 40,41, 37,59, 36,74, 36,52, 36,15, 35,86, 33,99, 33,71, 33,12, 31,32, 29,17, 24,58, 23,41, 21,75, 18,88, 18,00, 12,23. Yield: 76%.

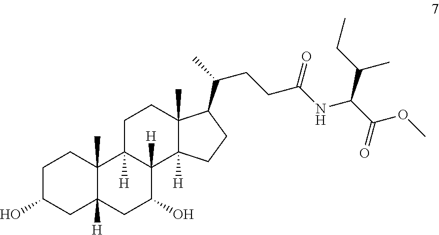

Methyl-L-isoleucinate chenodeoxycholate 7 (C₃₁H₅₃NO₅). NMR ¹H (400 MHz, CD₃OD): δ (ppm)=4.39 (q, J₁=4.55 Hz, J₂=4.54 Hz, 1H), 3.78-3.67 (m, 1H), 2.29-1.06 (m, 33H), 0.95-0.86 (m, 13H), 0.64 (s, 3H). NMR ¹³C (101 MHz, CD₃OD): δ (ppm)=176.50, 174.57, 72.60, 68.73, 57.17, 55.63, 52.60, 51.87, 51.30, 43.48, 42.90, 41.10, 40.87, 40.54, 40.19, 36.61, 36.44, 36.01, 33.81, 33.61, 33.04, 31.19, 29.12, 25.78, 24.48, 23.44, 23.32, 21.79, 18.89, 13.05, 12.25. Yield: >95%.

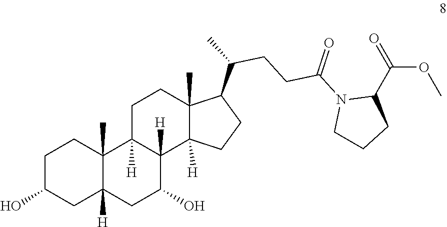

Methyl-L-prolinate chenodeoxycholate 8 (C₃₀H₄₉NO₅). NMR ¹H (400 MHz, CD₃OD): δ (ppm)=3.93-3.83 (m, 5H), 3.79-3.72 (m, 1H), 3.55-3.48 (m, 1H), 3.35 (q, J₁=4.41 Hz, J₂=4.39 Hz, 1H), 2,57-1.88 (m, 13H), 1.80-1.57 (m, 7H), 1.55-1.39 (m, 13H), 1.13 (d, J=2.17 Hz, 2H), 1.06 (s, 3H), 0.84 (s, 3H). NMR ¹³C (101 MHz, CD₃OD): δ (ppm)=174.56, 174.17, 72.52, 68.62, 59.95, 57.05, 55.57, 52.60, 51.26, 43.57, 43.42, 42.84, 40.83, 40.50, 40.14, 36.59, 35.96, 35.71, 33.76, 32.06, 31.19, 30.04, 29.09, 25.52, 24.45, 23.42, 21.63, 19.03, 13.00, 12.25. Yield: 89%.

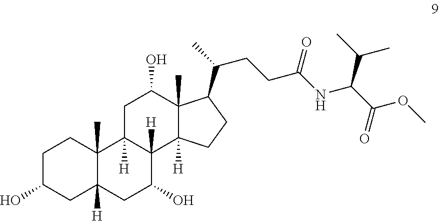

Methyl-L-valinate cholate 9 (C₃₀H₅₁NO₆). NMR ¹H (400 MHz, CD₃OD): δ (ppm)=4.34 (d, J=4.36 Hz, 1H), 4.01 (s, 1H), 3.89-3.74 (m, 7H), 3.31-3.22 (m, 3H), 2.42-1.56 (m, 21H), 1.20-1.04 (m, 5H), 1.03-0.96 (m, 10H), 0.76 (s, 3H). NMR ¹³C (63 MHz, CD₃OD): δ (ppm)=174.35, 172.75, 73.18, 71.93, 68.57, 57.32, 55.68, 52.16, 50.42, 46.72, 46.45, 43.62, 41.70, 41.48, 39.45, 35.41, 34.80, 33.20, 31.71, 31.11, 30.30, 27.59, 26.42, 23.26, 22.45, 18.98, 17.99, 17.42, 12.76, 12.48. Yield: >95%.

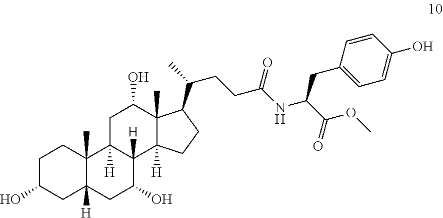

Methyl-L-tyrosinate cholate 10 (C₃₄H₅₁NO₇). NMR ¹H (400 MHz, CD₃OD): a (ppm)=7.07-7.04 (m, 2H), 6.76-6.72 (m, 2H), 4.65-4.59 (m, 1H), 3.99 (s, 1H), 3.85 (s, 1H), 3.45-3,39 (m, 3H), 3.12-3.07 (m, 1H), 2.97-2.86 (m, 2H), 2.38-2.24 (m, 5H), 2.18-1.03 (m, 30H), 0.74 (s, 3H). NMR ¹³C (63 MHz, CD₃OD): δ (ppm)=176.47, 173.63, 157.32, 131.08, 128.61, 116.18, 73.87, 72.72, 68.88, 55.30, 52.59, 49.85, 47.89, 47.33, 43.05, 42,82, 40,86, 40,33, 37,56, 36,69, 36,43, 35,79, 33,65, 33,08, 31,10, 29,49, 28,59, 27,72, 24,19, 23,22, 17,69, 13,04. Yield: 31%.

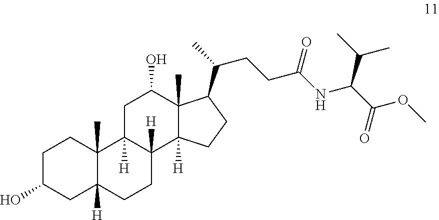

Methyl-L-valinate deoxycholate 11 (C₃₀H₅₁NO₅). NMR ¹H (400 MHz, CD₃OD): δ (ppm)=4.34 (d, J=4.35 Hz, 1H), 4.01 (s, 1H), 3.81-3.75 (m, 4H), 3.58-3.54 (m, 1H), 3.39 (s, 7H), 3,27 (q, J₁=3.29 Hz, J₂=3.28 Hz, 2H), 2.41-2.14 (m, 3H), 1.94-1.80 (m, 7H), 1.66-1.31 (m, 22H), 0.76 (s, 3H). NMR ¹³C (63 MHz, CD₃OD): δ (ppm)=176.78, 173.54, 73.75, 72.34, 59.18, 55.70, 52.35, 49.86, 49.11, 48.03, 47.47, 43.67, 37.34, 36.72, 36.42, 35.20, 34.68, 33.16, 31.56, 31.00, 29.85, 28.60, 28.38, 27.41, 24.83, 23.79, 19.53, 18.68, 17.70, 13.26. Obtained after purification by silica gel chromatography (eluent: CH₂Cl₂ then CH₂Cl₂/MeOH (9/1). Yield: >95%.

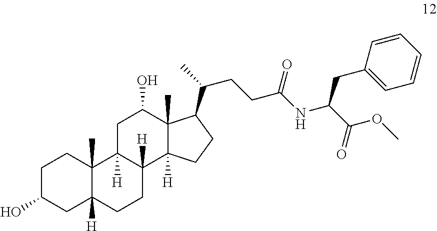

Methyl-L-phenylalaninate deoxycholate 12 (C₃₄H₅₁NO₅). NMR¹ H (250 MHz, CD₃OD): δ (ppm)=7.45-7.30 (m, 5H), 5.48 (s, 1H), 3.96 (s, 1H), 3.77-3.66 (m, 3H), 3.36 (s, 1H), 3.22 (q, J₁=3.23 Hz, J₂=3.20 Hz, 2H), 2.40-2.16 (m, 2H), 2.02-1.09 (m, 33H), 0.68 (s, 3H). NMR¹³ C (63 MHz, CD₃OD): δ (ppm)=176.27, 172.55, 137.35, 129.83, 129.48, 128.66, 73.85, 72.38, 58.03, 55.67, 52.96, 49.85, 49.12, 47.42, 43.66, 43.42, 37,28, 37,07, 36,62, 36,32, 35,16, 34,65, 33,40, 32,93, 30,94, 29,77, 28,56, 28,31, 27,37, 24,80, 23,72, 17,60, 13,21, 13,07. Obtained after purification by silica gel chromatography (eluent: CH₂Cl₂ then CH₂Cl₂/MeOH (9/1). Yield: 100%.

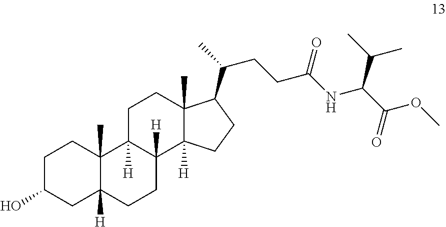

Methyl-L-valinate lithocholate 13 (C₃₀H₅₁NO₄). NMR¹ H (400 MHz, CD₃OD): δ (ppm)=4.31 (d, J=4.32 Hz, 1H), 3.73 (s, 3H), 3.59-3.52 (m, 1H), 3.37 (s, 2H), 2.38-0.93 (m, 41H), 0.71 (s, 3H). NMR ¹³C (101 MHz, CD₃OD): δ (ppm)=177.07, 173.79, 72.42, 59.28, 57.94, 57.51, 52.39, 43.93, 43.54, 41.89, 41.55, 37.24, 37.17, 36.83, 36.48, 35.68, 33.63, 33.25, 31.66, 31.19, 29.27, 28.36, 27.66, 25.26, 23.93, 21.95, 19.50, 18.87, 18.63, 12.49. Yield: >95%.

### B. Oppenauer Oxidation Procedure

### Synthesis of Alcohol 14

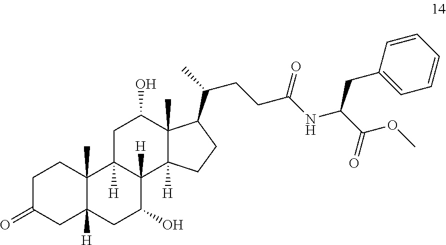

Methyl-L-phenylalaninate 3-oxo-cholate 14. In a 10-20 mL microwave reactor, 299 mg of methyl-L-phenylalaninate cholate (0.52 mmol) and 2 equivalents of aluminum tri-sec-butylate (274 mg, 1.1 mmol) dissolved in 10 mL of toluene and 6 mL of acetone are introduced. The reactor is sealed and placed in a Biotage Initiator+microwave system. The reaction is carried out under microwave irradiation (400 Watt) at 150° C., for 10 min using a normal mode and a 20-second pre-agitation. When the reaction is complete, 5 mL of NH₄·OH is added. After 15 min stirring, the mixture is filtered over Celite, rinsed with CH₂Cl₂ and concentrated in vacuo. Methyl-L-phenylalaninate 3-oxo-cholate 14 is thus obtained without purification, as a yellow oil in 81% yield. Methyl-L-phenylalaninate 3-oxo-cholate 14 (C₃₄H₄₉NO₆). NMR ¹H (101 MHz, CDCl₃): δ (ppm)=7.23-6.88 (m, 3H), 6.80-6.57 (m, 2H), 4.95 (s, 1H), 4.79 (s, 1H), 4.57 (m, 1H), 4.24-3.74 (m, 4H), 3.65 (s, 2H), 3.31 (s, 4H), 3.24-2.98 (m, 2H), 2.85-2.35 (m, 6H), 2.24-1.45 (m, 20H), 0.68 (s, 3H). NMR¹³C (400 MHz, CDCl₃): δ (ppm)=216.26, 176.49, 173.69, 157.32, 131.10, 128.66, 116.23, 73.73, 68.70, 55.32, 52.60, 47.49, 46.55, 44.71, 42.89, 40.87, 37,74, 37,58, 36,94, 36,71, 35,96, 35,80, 35,01, 33,73, 33,07, 29,82, 29,57, 28,33, 26,25, 24,12, 22,15, 17,72, 13,07, 9,75.

### B. 1. Application to the Synthesis of Ketosterols

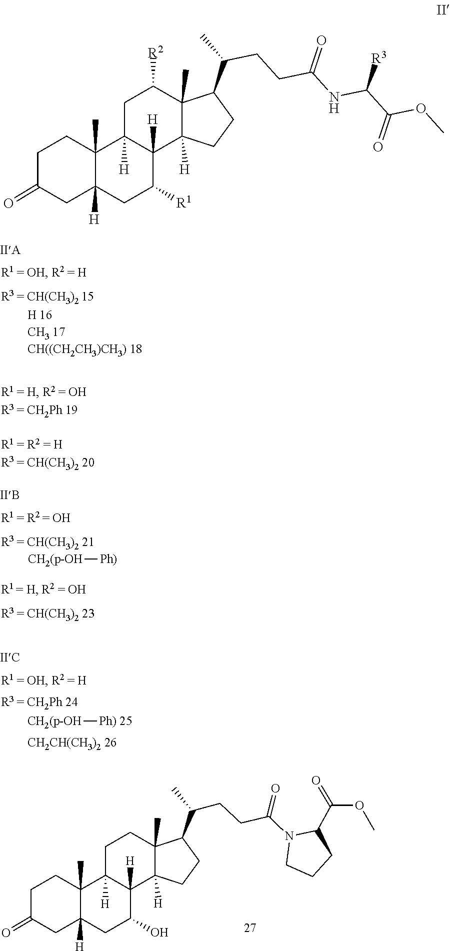

Ketosterols II′ were prepared from the corresponding alcohols I′ previously obtained. Three different procedures were used to obtain the products II. The first resulted in ketosterols II′A, the second in products II′B and the last in intermediates II′C.

### B.1.a. Procedure to Obtain Ketosterols II′A

The procedure to obtain ketosterols II′A is the same for all these products and is detailed below for the synthesis of compound 15.

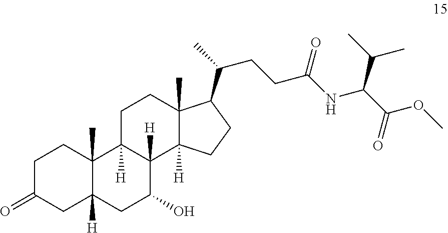

Methyl-L-valinate 3-oxo-chenodeoxycholate 15. In a 10-20 mL microwave reactor, 250 mg of methyl-L-valinate chenodeoxycholate 1 (0.451 mmol) and 292 mg of aluminum tri-ethanolate (1.81 mmol) dissolved in 10 mL of toluene and 6 mL of acetone are introduced. The reactor is sealed and placed in a Biotage Initiator+microwave system. The reaction is carried out under microwave irradiation (400 Watt) at 150° C., for 1 h using a normal mode and a 20-second pre-agitation. When the reaction is complete, 5 mL of a 2 N sulfuric acid solution is added and the medium is stirred for 15 min. The phases are separated, the organic phase is washed with 5 mL of an aqueous sodium bicarbonate solution (10%) and the aqueous phases are extracted twice with 5 mL of CH₂Cl₂. The combined organic phases are dried over anhydrous sodium sulphate and filtered. The crude product thus obtained is purified by chromatography on silica gel (eluent: PET then PET/EtOAc (8/2) then PET/EtOAc (1/1)). Methyl-L-valinate 3-oxo-chenodeoxycholate 15 was obtained as a yellow oil in 66% yield. Methyl-L-valinate 3-oxo-chenodeoxycholate 15 (C₃₀H₄₉NO₅). NMR ¹H (300 MHz, CD₃OD): δ (ppm)=4.39 (d, J=4.39 Hz, 1H), 3.99-3.41 (m, 6H), 2.58-1.19 (m, 27H), 1.11-1.01 (m, 12H), 0.81-0.76 (m, 3H). NMR ¹³C (75 MHz, CD₃OD): δ (ppm)=216.05, 176.79, 173.66, 68.74, 59.12, 57.36, 52.40, 51.42, 46.57, 44.77, 43.67, 40.91, 40.67, 37.94, 37.70, 36.82, 36.38, 35.12, 34.50, 33.63, 33.23, 31.63, 29.56, 29.22, 24.56, 22.42, 22.14, 19.54, 18.66, 12.31.

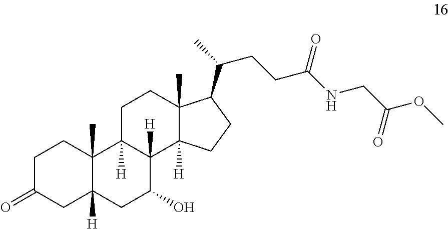

Methyl-L-glycinate 3-oxo-chenodeoxycholate 16 (C₂₇H₄₃NO₅). NMR ¹H (400 MHz, CD₃OD): δ (ppm)=3.97-3.89 (m, 2H), 3.54 (t, J=3.56 Hz, 1H), 3.39 (s, 2H), 2.55-1.77 (m, 14H), 1.66-1.14 (m, 21H), 0.79-0.75 (m, 3H). NMR¹³C (101 MHz, CD₃OD): δ (ppm)=216.11, 177.06, 171.32, 68.78, 62.13, 57.32, 51.43, 46.58, 44.79, 43.67, 42.00, 40.91, 40.67, 37.94, 37.70, 36.79, 36.39, 35.11, 34.50, 33.70, 33.06, 29.21, 24.55, 22.39, 22.13, 18.94, 12.28. Yield: 29%.

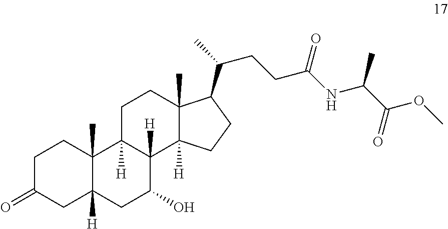

Methyl-L-alaninate 3-oxo-chenodeoxycholate 17 (C₂₈H₄₅NO₅). NMR ¹H (400 MHz, CD₃OD): δ (ppm)=4.44-4.36 (m, 1H), 4.22-4.10 (m, 2H), 3.88 (s, 1H), 3.74 (s, 2H), 3.52 (t, J=3.53 Hz, 1H), 2.50-1.75 (m, 13H), 1.65-1.13 (m, 22H), 0.77 (s, 3H). NMR ¹³C (101 MHz, CD₃OD): δ (ppm)=216.24, 176.50, 174.79, 68.85, 65.22, 57.39, 52.68, 51.47, 46.60, 44.83, 43.70, 40.94, 40.70, 37.95, 37.71, 36.80, 36.42, 35.12, 34.54, 33.67, 33.09, 29.24, 24.56, 22.37, 22.14, 18.94, 17.37, 12.26 The product was obtained after purification by chromatography on silica gel (PET then PET/EtOAc (1/1) then PET/EtOAc (3/7)). Yield: 42%.

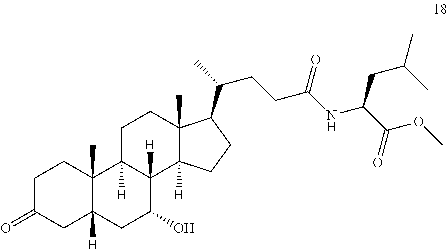

Methyl-L-leucinate 3-oxo-chenodeoxycholate 18 (C₃₁H₅₁NO₅). NMR ¹H (400 MHz, CD₃OD): δ (ppm)=4.39 (d, J=4.38 Hz, 1H), 3.89-3.87 (m, 1H), 3.52 (t, J=3.53 Hz, 1H), 2.53-1.13 (m, 34H), 1.03-0.88 (m, 11H), 0.76 (s, 3H). NMR ¹³C (101 MHz, CD₃OD): δ (ppm)=216.25, 176.83, 173.76, 68.84, 58.17, 57.40, 52.35, 51.47, 46.60, 44.83, 43.71, 40.94, 40.69, 38.22, 37.96, 37.71, 36.84, 36.42, 35.12, 34.53, 33.64, 33.24, 29.25, 26.39, 24.56, 22.37, 22.14, 18.94, 16.01, 12.26, 11.66. The product was obtained after purification by chromatography on silica gel (PET then PET/EtOAc (1/1) then PET/EtOAc (3/7)). Yield: 43%.

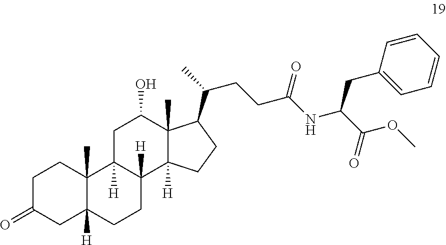

Methyl-L-phenylalaninate 3-oxodeoxycholate 19 (C₃₄H₄₉NO₅). NMR ¹H (400 MHz, CD₃OD): δ (ppm)=7.43-7.34 (m, 5H), 5.52 (s, 1H), 4.17-4.09 (m, 2H), 3.72 (s, 2H), 2.85 (t, J=2.87 Hz, 1H), 2.60-2.51 (m, 1H), 2.42-2.35 (m, 1H), 2.29-2.21 (m, 1H), 2.13-1.79 (m, 9H), 1.73-1.51 (m, 6H), 1.45-0.88 (m, 17H), 0.76 (s, 3H). NMR ¹³C (101 MHz, CD₃OD): δ (ppm)=216.09, 176.33, 172.65, 137.52, 129.87, 129.51, 128.78, 73.86, 65.20, 65.14, 58.11, 52.95, 48.15, 47.63, 45.82, 43,17, 38,00, 37,85, 36,99, 36,74, 35,53, 34,74, 33,53, 33,01, 30,07, 28,63, 27,68, 26,65, 24,81, 22,74, 17,68, 16,44, 16,38, 13,24. The product was obtained after purification by chromatography on silica gel (PET then PET/EtOAc (1/1) then PET/EtOAc (3/7)). Yield: 44%.

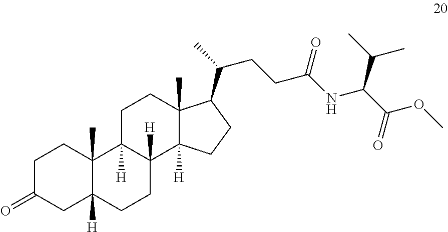

Methyl-L-valinate 3-oxo-lithocholate 20 (C₃₀H₄₉NO₄). NMR ¹H (400 MHz, CD₃OD): δ (ppm)=4.33 (d, J=4.33 Hz, 1H), 3.74 (s, 3H), 2.85 (t, J=2.85 Hz, 1H), 2.52-1.12 (m, 38H), 1.07 (s, 3H), 0.76 (s, 3H). NMR ¹³C (101 MHz, CD₃OD): δ (ppm)=216.04, 176.93, 173.74, 59.21, 57.57, 57.51, 52.39, 45.91, 43.94, 43.23, 41.74, 41.32, 38.17, 37.95, 36.92, 36.82, 36.01, 33.68, 33.26, 31.66, 29.26, 27.78, 26.87, 25.24, 23.04, 22.31, 19.52, 18.91, 18.64, 12.55. The product was obtained after purification by chromatography on silica gel (PET then PET/EtOAc (7/3)). Yield: 34%.

### B.1.b. Procedure to Obtain Ketosterols II′B

The procedure to obtain ketosterols II′B is the same for all these products, except for the reaction times which vary from one product to another. The general procedure is detailed below, for the synthesis of compound 23.

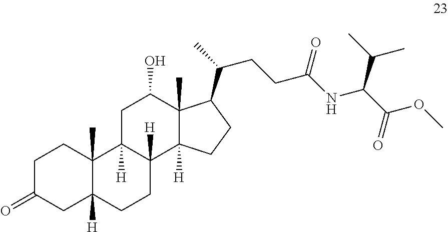

Methyl-L-valinate 3-oxo-deoxycholate 23. In a 10-20 mL microwave reactor, 250 mg of methyl-L-valinate deoxycholate 11 (0.494 mmol) and 487 mg of aluminum tri-sec-butylate (1.98 mmol) dissolved in 10 mL of toluene and 6 mL of acetone are introduced. The reactor is sealed and placed in a Biotage Initiator+microwave system. The reaction is carried out two successive times, under microwave irradiation (400 Watt) at 150° C., for 30 min using a normal mode and a 20-second pre-agitation. When the second reaction is completed, 5 mL of NH₄·OH is added. After 15 min of stirring, the mixture is filtered over Celite, rinsed with CH₂C₂ and concentrated under vacuum. The crude product thus obtained is purified by chromatography on silica gel (eluent: PET then PET/EtOAc (1/1) then EtOAc). Methyl-L-valinate 3-oxo-deoxycholate 23 was obtained as a yellow oil in 32% yield. Methyl-L-valinate 3-oxodeoxycholate 23 (C₃₀H₄₉NO₅). NMR ¹H (250 MHz, CD₃OD): δ (ppm)=4.28 (d, J=4.30 Hz, 1H), 4.01 (s, 1H), 3.70 (s, 3H), 2.82 (t, J=2.82 Hz, 1H), 2.59-1.21 (m, 29H), 1.08-0.92 (m, 11H), 0.73 (s, 3H). NMR ¹³C (63 MHz, CD₃OD): δ (ppm)=216.09, 176.98, 173.74, 73.88, 59.17, 52.41, 49.19, 48.17, 47.64, 45.84, 43.18, 38.01, 37.86, 37.00, 36.84, 35.54, 34.74, 33.67, 33.25, 31.64, 30.09, 28.67, 27.69, 26.66, 24.81, 22.74, 19.53, 18.64, 17.70, 13.23.

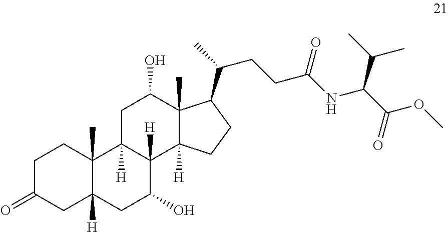

Methyl-L-valinate 3-oxo-cholate 21. The reaction is carried out under microwave irradiation (400 Watt) at 150° C., for 1 hour using a normal mode and a 20 second pre-agitation. Methyl-L-valinate 3-oxo-cholate 21 (C₃₀H₄₉NO₆). NMR ¹H (250 MHz, CD₃OD): δ (ppm)=4.89 (s, 7H), 4.79 (s, 1H), 4.28 (d, J=4.27 Hz, 1H), 3.99 (m, 1H), 3.83 (m, 1H), 3.71 (s, 3H), 3.61-3.46 (m, 1H), 3.33 (s, 1H), 2.64-1.12 (m, 30H), 0.73 (s, 3H). NMR ¹³C (63 MHz, CD₃OD): δ (ppm)=216.57, 177.10, 173.80, 73.85, 68.78, 59.24, 52.42, 48.11, 47.58, 44.90, 43.00, 40.96, 37.83, 37.64, 36.92, 36.08, 35.05, 33.67, 33.28, 31.65, 29.84, 28.74, 28.37, 24.16, 22.13, 19.51, 18.63, 17.71, 13.03. The product was obtained after purification by chromatography on silica gel (CH₂C₂ then CH₂C₂/EtOAc (1/1) then EtOAc). Yield: 21%.

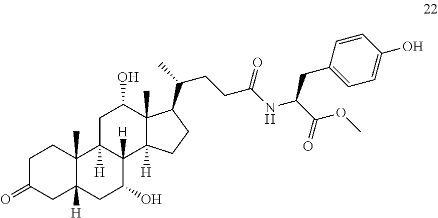

Methyl-L-tyrosinate 3-oxo-cholate 22 The reaction is carried out three times successively, under microwave irradiation (400 Watt) at 150° C., for 10 min using a normal mode and a pre-agitation of 20 seconds. Methyl-L-tyrosinate 3-oxo-cholate 22 (C₃₄H₄₉NO₇). The product is used crude for the reductive amination reaction.

### B.1.c. Procedure to Obtain II′C Ketosterols

The procedure to obtain ketosterols II′C is the same for all these products and is detailed below for the synthesis of compound 24.

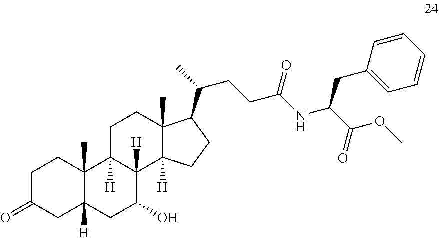

Methyl-L-phenylalaninate 3-oxo-chenodeoxycholate 24 In a 10-20 mL microwave reactor, 200 mg of methyl-L-phenylalaninate chenodeoxycholate 2 (0.361 mmol) and 295 mg of aluminum triethanolate (1.44 mmol) dissolved in 8 mL of toluene and 4 mL of acetone are introduced. The reactor is sealed and placed in a Biotage Initiator+microwave system. The reaction is carried out under microwave irradiation (400 Watt) at 150° C., for 1 h using a normal mode and a pre-agitation of 20 seconds. This manipulation is repeated on 10 different batches. Once the 10 reactions have been completed, they are assembled and 50 mL of a 2 N sulfuric acid solution is added. The medium is stirred for 15 min and the phases are separated. The organic phase is washed with 50 mL of an aqueous sodium bicarbonate solution (10%), the aqueous phases are extracted twice with 50 mL of CH₂Cl₂ and a washing is performed with 50 mL of a saturated aqueous sodium chloride solution. The combined organic phases are dried over anhydrous sodium sulphate and filtered. The crude product thus obtained is purified by chromatography on silica gel (eluent: PET then PET/EtOAc (8/2) then PET/EtOAc (1/1)). Methyl-L-phenylalaninate 3-oxo-chenodeoxycholate 24 was obtained as a yellow oil in 51% yield. Methyl-L-phenylalaninate 3-oxo-chenodeoxycholate 24 (C₃₄H₄₉NO₅). NMR ¹H (300 MHz, CD₃OD): δ (ppm)=7.49-7.37 (m, 5H), 5.61 (s, 1H), 3.95-3.93 (m, 1H), 3.77 (s, 3H), 3.60 (t, J=3.62 Hz, 1H), 2.73-0.94 (m, 35H), 0.82-0.75 (m, 3H). NMR ¹³C (75 MHz, CD₃OD): δ (ppm)=215.95, 176.04, 172.52, 137.45, 129.81, 129.43, 128.69, 68.65, 57.97, 57.22, 52.95, 51.34, 46.52, 44.66, 43.59, 42,12, 40,82, 40,60, 37,87, 37,66, 36,68, 36,31, 35,05, 34,42, 33,43, 29,53, 29,15, 24,52, 22,42, 22,09, 18,98, 18,15, 15,11, 12,34.

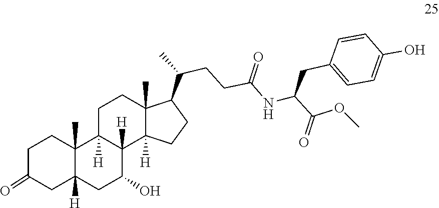

Methyl-L-tyrosinate 3-oxo-chenodeoxycholate 25 (C₃₄H₄₉NO₆). NMR ¹H (400 MHz, CD₃OD): δ (ppm)=6.90 (d, J=6.90 Hz, 2H), 6.59 (d, J=6.60 Hz, 2H), 3.73 (s, 1H), 3.38 (t, J=3.38 Hz, 1H), 3.24 (s, 1H), 2.97-2.71 (m, 3H), 2.37-0.99 (m, 32H), 0.90 (s, 2H), 0.82 (m, 3H), 0.59 (s, 3H). NMR ¹³C (101 MHz, CD₃OD): δ (ppm)=216.11, 176.39, 173.64, 157.23, 131.11, 128.61, 116.17, 68.70, 57.16, 55.24, 52.61, 51.31, 46.50, 44.61, 43,56, 40,78, 40,54, 37,83, 37,65, 37,55, 36,66, 36,28, 35,01, 34,41, 33,63, 33,03, 29,13, 24,49, 22,37, 22,05, 18,91, 12,30. Yield: 47%.

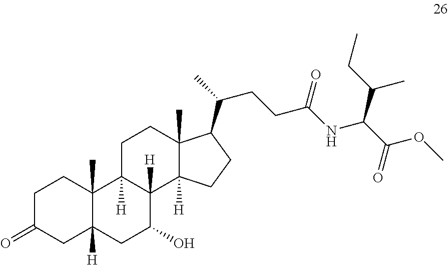

Methyl-L-isoleucinate 3-oxo-chenodeoxycholate 26 (C₃₁H₅₁NO₅). NMR ¹H (400 MHz, CD₃OD): δ (ppm)=4.49-4.44 (m, 1H), 4.21-4.10 (m, 1H), 3.89-3.87 (m, 1H), 3.52 (t, J=3.53 Hz, 1H), 2.53-1.13 (m, 34H), 1.03-0.88 (m, 10H), 0.77 (s, 3H). NMR ¹³C (101 MHz, CD₃OD): δ (ppm)=216.25, 176.81, 174.77, 68.83, 57.40, 52.61, 52.04, 51.47, 46.59, 44.83, 43.71, 41.34, 40.94, 40.69, 37.96, 37.71, 36.78, 36.42, 35.12, 34.53, 33.73, 33.20, 29.25, 26.01, 24.57, 23.34, 22.37, 22.14, 21.78, 18.92, 12.27. Yield: 50%.

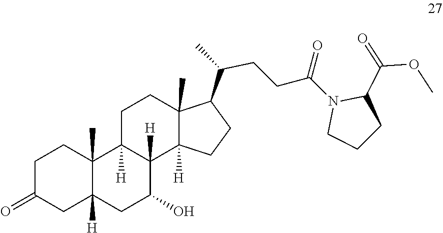

Methyl-L-prolinate 3-oxo-chenodeoxycholate 27 (C₃₀H₄₇NO₅). NMR ¹H (400 MHz, CD₃OD): δ (ppm)=4.46-4.41 (m, 1H), 3.90-3.80 (m, 1H), 3.74 (s, 2H), 3.71-3.49 (m, 2H), 3.38 (s, 1H), 2.54-1.13 (m, 33H), 1.07-0.89 (m, 7H), 0.78-0.72 (m, 3H). NMR ¹³C (101 MHz, CD₃OD): δ (ppm)=216.19, 174.96, 174.46, 68.82, 60.19, 57.34, 52.68, 51.46, 46.59, 44.82, 43.70, 43.67, 40.93, 40.70, 37.96, 37.71, 36.83, 36.42, 35.12, 34.53, 32.24, 32.06, 30.24, 29.25, 25.73, 24.58, 22.38, 22.14, 19.06, 12.27. Yield: 46%.

### C. Stereoselective Reductive Amination Procedure

All the products presented in this section were synthesized from ketosterol II′ and the corresponding polyamine, following the same procedure detailed below for the synthesis of methyl-L-valinate 30-norspermidino-chenodeoxycholate 007.

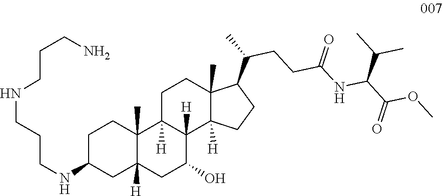

Methyl-L-valinate 3β-norspermidino-chenodeoxycholate 007. In a 50 mL flask fitted with a magnetic stirring bar, 870 mg of methyl-L-valinate 3-oxo-chenodeoxycholate 15 (1.74 mmol) is dissolved in 30 mL of MeOH. Subsequently, 3 equivalents of norspermidine (0.73 mL, 5.21 mmol) and 4 equivalents of titanium tetra-isopropylate (2.06 mL, 6.96 mmol) are added. After 12 h stirring at 20° C., the flask is placed at −78° C. and 4 equivalents of sodium borohydride (50 mg, 1.36 mmol) are added while stirring. The reaction medium is left to stir at this temperature for 2 h. The reaction medium was then transferred to an ice bath at 0° C., where a slow ascent to 20° C. was carried out for 5 h with stirring. Once the temperature is reached, 8.7 mL of water is added to neutralize the reaction. After 30 min of further stirring, the mixture was filtered over Celite and rinsed with MeOH, CH₂Cl₂ and EtOAc before being concentrated in vacuo.

The crude product thus obtained is purified by chromatography on silica gel (eluent: MeOH then CH₂Cl₂/MeOH/NH₄·OH (7/3/1)). Methyl-L-valinate 30-norspermidino-chenodeoxycholate 007 is obtained, in the form of a yellow oil with a yield of 20% (mixture of two diastereomers (B/a) in a ratio (96/4), herein “007-a1” as a mixture). Methyl-L-valinate 3β-norspermidino-chenodeoxycholate 007 (C₃₆H₆₆N₄O₄). NMR ¹H (300 MHz, CD₃OD): δ (ppm)=4.30 (d, J=4.31 Hz, 1H), 3.80 (s, 1H), 3.74 (s, 1H), 3.71 (s, 2H), 2.82-2.64 (m, 7H), 2.52-1.10 (m, 35H), 1.02-0.91 (m, 14H), 0.70 (s, 3H). NMR ¹³C (75 MHz, CD₃OD): δ (ppm)=176.94, 173.74, 68.91, 59.29, 59.23, 57.45, 52.40, 51.55, 48.86, 48.19, 45.58, 43.68, 43.52, 41.08, 40.81, 40.56, 37.29, 36,99, 36,89, 36,63, 35,91, 34,06, 33,65, 33,28, 32,98, 31,65, 29,66, 29,28, 28,03, 24,62, 23,58, 21,78, 19,53, 18,94, 18,66, 12,25. MS (ESI⁺): m/z 619.5157 ([M+H]⁺).

Compound 007-a1 (β/α: 96/04) as prepared with the above-described method was used for manufacturing the corresponding hydrochloric salt S007 (P/a: 96/04) as described hereinafter (section “D. Preparation of the different salts”).

Others batches were prepared following the above-described method and yielded compound 007 as mixtures presenting slightly different stereochemical ratios, which are noted respectively 007-a2 (β/α: 94/06) and 007-a3 (β/α: 90/10). These compounds were used for manufacturing the corresponding hydrochloric salts S019 ((β/α: 94/06) and S021 (β/α: 90/10) as described hereinafter (section “D. Preparation of the different salts”).

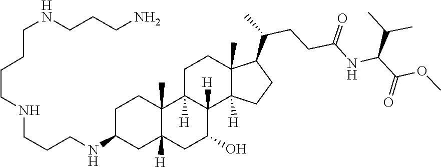

Methyl-L-valinate 3β-spermino-chenodeoxycholate 001 (C₄₀H₇₅N₅O₄). NMR ¹H (250 MHz, CD₃OD): δ (ppm)=4.95-4.68 (m, 9H), 4.24 (d, J=3.34 Hz, 1H), 3.89-3.64 (m, 3H), 3.45-3.17 (m, 2H), 2.96-2.53 (m, 15H), 2.42-0.87 (m, 42H), 0.73 (s, 3H). NMR ¹³C (63 MHz, CD₃OD): δ (ppm)=176.89, 173.73, 68.84, 59.25, 57.50, 52.39, 51.57, 50.20, 49.85, 48.09, 45.48, 43.69, 43.45, 41.09, 40.81, 40.46, 36.88, 36,58, 35,87, 34,08, 33,70, 33,29, 32,69, 32,12, 31,64, 29,27, 28,88, 27,91, 27,68, 24,61, 23,53, 21,79, 19,53, 18,96, 18,68, 18,47, 12,26. MS (ESI⁺): m/z 690.6 ([M+H]⁺). Yield: 41%. Mixture of two diastereomers (B/α) in a ratio (95/05).

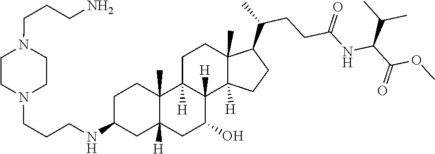

Methyl-L-valinate 3β-(1,4-bis(3-aminopropyl)piperazine)-chenodeoxycholate 002 (C₄₀H₇₃N₅O₄). NMR ¹H (400 MHz, CD₃OD): δ (ppm)=4.33 (d, J=4.35 Hz, 1H), 3.83 (s, 1H), 3.75 (s, 1H), 3.39 (s, 4H), 2.96-1.93 (m, 27H), 1.87-0.94 (m, 36H), 0.74 (s, 3H). NMR ¹³C (63 MHz, CD₃ OD): δ (ppm)=176.88, 173.69, 68.69, 59.22, 58.97, 57.77, 57.53, 57.23, 54.06, 53.75, 52.42, 51.60, 49.85, 45.93, 43.66, 43.23, 41.13, 40.78, 36,88, 36,48, 35,83, 35,80, 34,10, 33,66, 33,29, 31,62, 29,33, 29,27, 29,16, 29,03, 27,28, 24,60, 23,44, 21,82, 21,74, 19,56, 18,97, 18,68, 12,29. MS (ESI⁺): m/z 688.6 ([M+H]⁺). Yield: 90%. Mixture of two diastereomers (β/α) in a ratio (95/05).

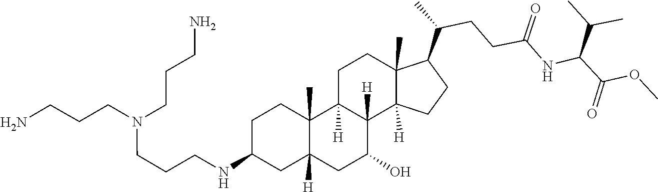

Methyl-L-valinate 3β-(Tris(3-aminopropyl)amine)-chenodeoxycholate 003 (C₃₉H₇₃N₅O₄). NMR ¹H (400 MHz, CD₃OD): δ (ppm)=3.84 (s, 1H), 3.39 (s, 10H), 3.28 (m, 1H), 2.83-2.68 (m, 6H), 2.55 (s, 4H), 2.39-0.76 (m, 48H), 0.75 (s, 3H). NMR ¹³C (63 MHz, CD₃ OD): δ (ppm)=176.94, 173.74, 68.87, 59.36, 59.24, 57.43, 54.06, 53.32, 52.63, 52.63, 52.44, 51.55, 49.43, 49.14, 48.47, 48.13, 45.92, 43.66, 41,08, 40,85, 40,77, 37,11, 36,89, 36,61, 34,06, 33,63, 33,29, 31,63, 30,05, 29,28, 27,89, 26,87, 24,64, 23,58, 21,79, 19,55, 18,95, 18,67, 12,27. MS (ESI⁺): m/z 676.5735 ([M+H]⁺). Yield: 51%. Mixture of two diastereomers (β/α) in a ratio (95/05).

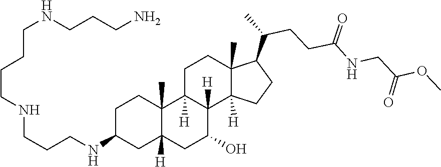

Methyl-L-glycinate 3β-spermino-chenodeoxycholate 004 (C₃₇H₆₉N₅O₄). NMR ¹H (250 MHz, CD₃OD): δ (ppm)=4.79 (s, 1H), 3.94-3.68 (m, 3H), 3.40-3.17 (m, 5H), 2.97-2.59 (m, 11H), 2.32-2.17 (m, 4H), 1.96-1.58 (m, 17H), 1.45-1.13 (m, 16H), 1.02-0.86 (m, 9H), 0.67 (s, 3H). NMR ¹³C (75 MHz, CD₃OD): δ (ppm)=177.36, 172.63, 69.02, 57.35, 51.57, 49.38, 47.45, 46.66, 44.65, 43.68, 43.17, 41.07, 40.77, 39.71, 37.44, 36.99, 36.54, 36,39, 36,21, 35,92, 34,06, 33,87, 33,00, 31,36, 29,30, 28,51, 28,37, 26,53, 26,37, 26,06, 25,34, 24,63, 23,39, 23,30, 21,78, 18,97, 12,19. MS (ESI⁺): m/z 648.5 ([M+H]⁺). The product was obtained after purification by chromatography on silica gel (MeOH then CH₂C₂/MeOH/NH₄·OH (7/3/2)). Yield: 14% (mixture of two diastereomers (β/α) in a ratio (95/05) herein “004-a1” as a mixture). The corresponding hydrochloric salt S004 (β/α: 95/05) was prepared starting from 004-a1 as described hereinafter (section “D. Preparation of the different salts”).

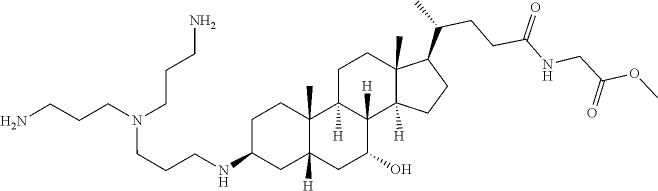

Methyl-L-glycinate 3β-(Tris(3-aminopropyl)amine)-chenodeoxycholate 005 (C₃₆H₆₇N₅O₄). NMR ¹H (400 MHz, CD₃OD): δ (ppm)=3.84 (s, 3H), 3.63 (t, J=3.64 Hz, 1H), 3.50-3.37 (m, 2H), 3.33-3.26 (m, 3H), 3.12 (s, 1H), 2.82-2.51 (m, 10H), 2.38-2.21 (m, 4H), 2.07-1.16 (m, 33H), 1.10-0.93 (m, 7H), 0.75 (s, 3H). NMR ¹³C (101 MHz, CD₃OD): δ (ppm)=177.30, 171.78, 72.87, 69.04, 57.38, 57.35, 52.86, 52.24, 51.58, 49.85, 49.50, 49.28, 49.07, 46.06, 44.76, 43.70, 43.20, 41,08, 40,84, 40,50, 38,65, 36,96, 36,57, 36,23, 35,94, 34,08, 33,87, 33,01, 29,50, 29,27, 27,48, 24,63, 23,39, 21,79, 18,95, 12,19. MS (ESI⁺): m/z 634.5 ([M+H]⁺). Yield: 14%. Mixture of two diastereomers (β/α) in a ratio (95/05).

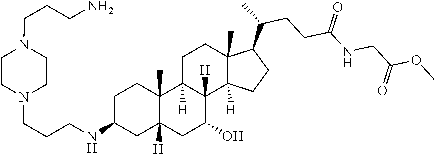

Methyl-L-glycinate 3β-(1,4-bis(3-aminopropyl)piperazine)-chenodeoxycholate 006 (C₃₇H₆₇N₅O₄). NMR ¹H (400 MHz, CD₃OD): δ (ppm)=3.84 (s, 3H), 3.46-3.40 (m, 2H), 3.33-3.26 (m, 2H), 2.91 (t, J=2.89 Hz, 1H), 2.77-2.19 (m, 20H), 2.08-1.14 (m, 31H), 1.09-0.95 (m, 8H), 0.73 (s, 3H). NMR ¹³C (101 MHz, CD₃OD): δ (ppm)=177.20, 171.71, 72.86, 69.02, 57.36, 57.29, 56.93, 54.87, 53.91, 53.87, 51.56, 49.85, 46.14, 43.69, 43.66, 43.20, 41.08, 40,94, 40,80, 40,50, 38,72, 36,94, 36,57, 36,22, 35,93, 34,07, 33,87, 32,98, 31,38, 29,82, 29,26, 27,26, 24,63, 23,40, 21,79, 18,95, 12,20. MS (ESI⁺): m/z 646.5 ([M+H]⁺). Yield: 17%. Mixture of two diastereomers (β/α) in a ratio (95/05).

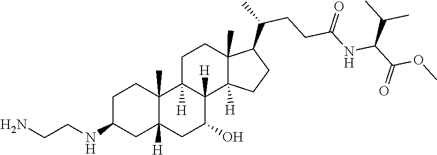

Methyl-L-valinate 3β-(ethylenediamine)-chenodeoxycholate 008 (C₃₂H₅₇N₃O₄). NMR ¹H (400 MHz, CD₃OD): δ (ppm)=4.37 (d, J=4.38 Hz, 1H), 3.87-3.85 (m, 1H), 3.78 (s, 2H), 3.42 (s, 1H), 2.91-2.69 (m, 4H), 2.55-0.97 (m, 45H), 0.76 (s, 3H). NMR ¹³C (101 MHz, CD₃OD): δ (ppm)=176.93, 173.75, 68.94, 59.38, 59.24, 57.45, 52.38, 51.54, 49.36, 43.70, 43.58, 41.82, 41.09, 40.84, 37.65, 37,08, 36,87, 36,65, 35,95, 34,07, 33,67, 33,27, 31,66, 29,26, 28,38, 24,62, 23,60, 21,79, 19,51, 18,96, 18,66, 12,26. MS (ESI⁺): m/z 548.4 ([M+H]⁺). Yield: 44%. Mixture of two diastereomers (β/α) in a ratio (95/05).

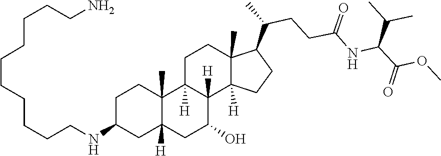

Methyl-L-valinate 3β-(1,10-diamino decane)-chenodeoxycholate 009 (C₄₀H₇₃N₃O₄). NMR ¹H (400 MHz, CD₃OD): δ (ppm)=4.36 (d, J=4.35 Hz, 1H), 3.92-3.78 (m, 1H), 3.76 (s, 2H), 2.89-2.57 (m, 4H), 2.41-1.49 (m, 24H), 1.38 (s, 19H), 1.25-0.91 (m, 19H), 0.75 (s, 3H). NMR ¹³C (75 MHz, CD₃OD): δ (ppm)=176.93, 173.73, 68.96, 59.38, 59.22, 57.43, 52.40, 51.52, 49.79, 48.20, 47.49, 43.70, 43.61, 42.56, 41.08, 40.83, 37.15, 36.88, 36.68, 35,96, 34,05, 33,79, 33,65, 33,28, 31,66, 30,67, 30,56, 30,37, 29,28, 28,52, 28,37, 28,04, 24,63, 23,65, 22,42, 21,81, 19,54, 18,97, 18,67, 12,28. MS (ESI⁺): m/z 660.6 ([M+H]⁺). The product was obtained after purification by chromatography on silica gel (MeOH then CH₂Cl₂/MeOH/NH₄·OH (7/3/0.5)). Yield: 32%. Mixture of two diastereomers (β/α) in a ratio (95/05).

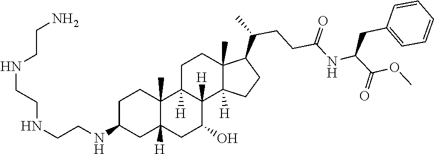

Methyl-L-phenylalaninate 3β-(triethylenetetramine)-chenodeoxycholate 010 (C₄₀H₆₇N₅O₄). NMR ¹H (300 MHz, CD₃OD): δ (ppm)=7.47-7.23 (m, 5H), 5.27 (s, 1H), 3.81-3.72 (m, 3H), 3.38 (s, 2H), 2.87-0.97 (m, 53H), 0.70 (s, 3H). NMR ¹³C (75 MHz, CD₃OD): δ (ppm)=176.86, 175.19, 141.85, 129.45, 129.25, 128.79, 128.43, 128.13, 68.84, 60.64, 60.16, 59.35, 57.45, 54.82, 54.02, 51.57, 51.24, 49.79, 49,41, 48,59, 46,11, 45,90, 43,69, 43,40, 41,26, 41,07, 40,82, 38,64, 36,79, 36,56, 35,82, 34,25, 34,07, 33,20, 29,27, 24,62, 23,49, 21,78, 18,97, 12,27. MS (ESI⁺): m/z 682.5 ([M+H]⁺). Yield: 28%. Mixture of two diastereomers (β/α) in a ratio (95/05).

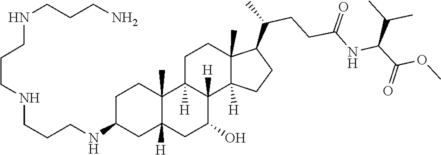

Methyl-L-valinate 3β-norspermino-chenodeoxycholate 011 (C₃₉H₇₃N₅O₄). NMR ¹H (400 MHz, CD₃OD): δ (ppm)=4.35 (d, J=4.35 Hz, 1H), 3.85 (s, 1H), 3.76 (s, 3H), 3.45-3.40 (m, 1H), 3.29-3.19 (m, 1H), 3.04-1.86 (m, 22H), 1.75-1.07 (m, 26H), 1.04-0.91 (m, 15H), 0.74 (s, 3H). NMR ¹³C (101 MHz, CD₃OD): δ (ppm)=177.02, 173.79, 68.96, 59.39, 59.16, 57.66, 56.40, 53.69, 52.52, 52.40, 52.25, 51.43, 49.82, 48.12, 46.34, 43.71, 43.45, 41.23, 41,00, 40,60, 37,04, 36,67, 34,12, 33,89, 33,69, 33,22, 31,72, 31,61, 29,38, 29,13, 24,61, 24,50, 23,45, 19,61, 19,39, 18,74, 18,54, 18,32, 12,29. MS (ESI⁺): m/z 676.6 ([M+H]⁺). Yield: 6%. Mixture of two diastereomers (β/α) in a ratio (95/05).

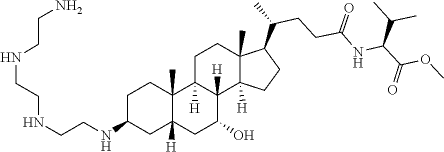

Methyl-L-valinate 3β-(triethylenetetramine)-chenodeoxycholate 012 (C₃₆H₆₇N₅O₄). NMR ¹H (300 MHz, CD₃OD): δ (ppm)=4.33-4.22 (m, 1H), 3.83 (s, 1H), 3.73 (s, 1H), 3.37 (s, 1H), 3.20-1.82 (m, 31H), 1.62-1.16 (m, 16H), 1.07-0.92 (m, 13H), 0.72 (s, 3H). NMR ¹³C (75 MHz, CD₃OD): δ (ppm)=175.97, 173.79, 68.78, 61.34, 59.29, 57.48, 53.91, 52.40, 51.62, 48.82, 48.62, 48.57, 43.70, 41.09, 40.79, 40.16, 38.05, 37,00, 35,70, 34,51, 34,10, 33,47, 32,69, 31,65, 29,30, 27,57, 26,09, 24,60, 23,32, 21,77, 20,24, 19,50, 18,97, 18,64, 18,44, 12,24. MS (ESI⁺): m/z 634.5 ([M+H]⁺). The product was obtained after purification by chromatography on silica gel (MeOH then MeOH/NH₄·OH (10/1)). Yield: 10%. Mixture of two diastereomers (β/α) in a ratio (95/05).

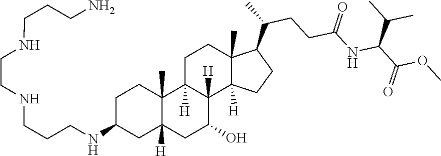

Methyl-L-valinate 3β-(Bis(3-aminopropyl)ethylenediamine)-N,N-diisopropyl-chenodeoxycholate 013 (C₃₈H₇₁N₅O₄). NMR ¹H (400 MHz, CD₃OD): δ (ppm)=4.30-4.20 (m, 1H), 3.81 (s, 1H), 3.71 (s, 1H), 3.35 (s, 4H), 3.18-2.70 (m, 13H), 2.51-0.90 (m, 48H), 0.70 (s, 3H). NMR ¹³C (101 MHz, CD₃OD): δ (ppm)=178.45, 176.10, 68.78, 61.21, 59.29, 57.47, 52.40, 51.63, 49.85, 47.20, 47.03, 46.65, 45.07, 43.70, 43.36, 43.00, 40.77, 39.09, 37,00, 36,33, 36,16, 35,48, 34,43, 34,06, 33,49, 32,55, 31,65, 29,31, 26,34, 24,58, 23,22, 21,79, 20,20, 19,50, 18,98, 18,64, 18,45, 12,22. MS (ESI⁺): m/z 662.6 ([M+H]⁺). Yield: 25%. Mixture of two diastereomers (β/α) in a ratio (95/05).

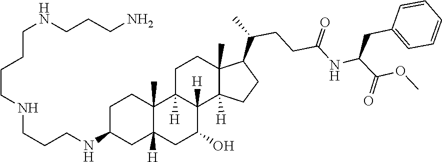

Methyl-L-phenylalaninate 3β-spermino-chenodeoxycholate 014 (C₄₄H₇₅N₅O₄). NMR ¹H (400 MHz, CD₃OD): δ (ppm)=7.48-7.16 (m, 5H), 3.81 (s, 1H), 3.35 (s, 5H), 3.24-2.92 (m, 14H), 2.48-0.86 (m, 47H), 0.68 (s, 3H). NMR ¹³C (101 MHz, CD₃OD): δ (ppm)=175.95, 173.81, 139.38, 130.52, 129.48, 129.32, 129.18, 128.47, 68.73, 59.28, 57.38, 56.61, 52.42, 51.58, 49.83, 47.84, 46.02, 45.85, 43.67, 42.99, 41,03, 40,76, 39,08, 37,89, 36,89, 36,32, 36,16, 35,50, 34,02, 33,83, 33,30, 31,65, 30,73, 29,26, 25,31, 25,11, 24,43, 23,23, 22,59, 22,06, 21,78, 19,36, 12,21. MS (ESI⁺): m/z 738.6 ([M+H]⁺). The product was obtained after purification by chromatography on silica gel (MeOH then MeOH/NH₄·OH (10/3)). Yield: 15%. Mixture of two diastereomers (β/α) in a ratio (95/05).

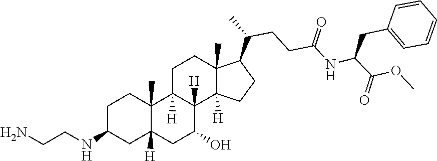

Methyl-L-phenylalaninate 3β-(ethylenediamine)-chenodeoxycholate 015 (C₃₆H₅₇N₃O₄). NMR ¹H (400 MHz, CD₃OD): δ (ppm)=7.29-7.12 (m, 5H), 4.49-4.46 (m, 1H), 3.78 (s, 1H), 3.51-3.18 (m, 3H), 3.08-2.48 (m, 7H), 2.36-0.89 (m, 37H), 0.65 (s, 3H). NMR ¹³C (101 MHz, CD₃OD): δ (ppm)=178.40, 175.66, 139.66, 130.61, 129.11, 127.29, 68.77, 59.28, 57.36, 57.26, 51.55, 49.85, 46.63, 43.66, 43.63, 43.22, 41.05, 40,77, 39,70, 39,39, 36,91, 36,51, 36,45, 35,71, 35,62, 34,34, 34,06, 33,30, 29,24, 26,69, 24,58, 23,37, 21,76, 18,94, 12,29, 12,26. MS (ESI⁺): m/z 596.4 ([M+H]⁺). The product was obtained after purification by chromatography on silica gel (MeOH then MeOH/NH₄·OH (10/0.25)). Yield: 21%. Mixture of two diastereomers (β/α) in a ratio (95/05).

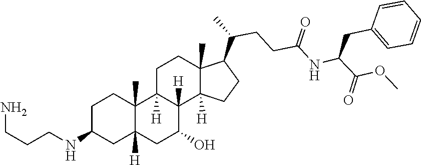

Methyl-L-phenylalaninate 3β-(1,3-diamino propane)-chenodeoxycholate 016 (C₃₇H₅₉N₃O₄). NMR ¹H (400 MHz, CD₃OD): δ (ppm)=7.32-7.13 (m, 5H), 4.52-4.47 (m, 1H), 3.82 (s, 1H), 3.36 (s, 3H), 3.29-2.64 (m, 8H), 2.48-0.91 (m, 38H), 0.68 (s, 3H). NMR ¹³C (101 MHz, CD₃OD): δ (ppm)=178.28, 175.73, 139.65, 130.61, 130.30, 129.52, 129.10, 127.28, 68.72, 59.35, 57.38, 56.61, 51.60, 49.85, 43.65, 42.99, 42.66, 41,06, 40,76, 39,34, 38,06, 36,92, 36,32, 35,49, 34,32, 34,08, 33,31, 29,24, 28,62, 25,81, 25,23, 24,56, 23,22, 21,76, 18,91, 17,31, 12,22. MS (ESI⁺): m/z 610.5 ([M+H]⁺). The product was obtained after purification by chromatography on silica gel (MeOH then MeOH/NH₄·OH (10/0.5)). Yield: 5%. Mixture of two diastereomers (β/α) in a ratio (95/05).

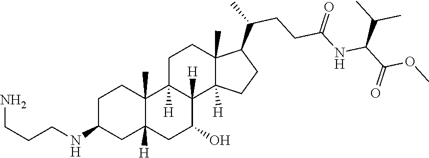

Methyl-L-valinate 3β-(1,3-diamino propane)-chenodeoxycholate 018 (C₃₃H₅₉N₃O₄). NMR ¹H (400 MHz, CD₃OD): δ (ppm)=4.33-4.22 (m, 1H), 3.84 (s, 1H), 3.73 (s, 1H), 3.43-3.37 (m, 3H), 3.20-2.75 (m, 6H), 2.52-0.95 (m, 44H), 0.73 (s, 3H). NMR ¹³C (101 MHz, CD₃OD): δ (ppm)=177.97, 176.25, 68.73, 60.98, 59.45, 57.53, 52.40, 51.61, 43.71, 43.02, 42.61, 41.07, 40.80, 37.92, 36.99, 36,17, 35,52, 34,35, 34,07, 33,87, 33,45, 32,34, 31,66, 29,31, 25,50, 25,15, 24,60, 23,24, 21,80, 20,12, 19,03, 18,52, 12,26. MS (ESI⁺): m/z 562.5 ([M+H]⁺). The product was obtained after purification by chromatography on silica gel (MeOH then MeOH/NH₄·OH (10/1)). Yield: 10%. Mixture of two diastereomers (β/α) in a ratio (95/05).

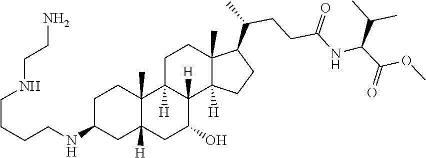

Methyl-L-valinate 3β-(diethylenetriamine)-chenodeoxycholate 020 (C₃₄H₆₂N₄O₄). NMR ¹H (400 MHz, CD₃OD): δ (ppm)=4.31 (d, J=4.31 Hz, 1H), 3.97-3.90 (m, 1H), 3.82 (s, 1H), 3.72 (s, 3H), 3.48-2.64 (m, 8H), 2.51-1.76 (m, 15H), 1.61-0.94 (m, 30H), 0.71 (s, 3H). NMR ¹³C (101 MHz, CD₃OD): δ (ppm)=177.04, 173.77, 68.95, 62.36, 59.29, 57.52, 52.42, 51.56, 51.19, 45.96, 45.79, 43.85, 43.67, 43.14, 41.03, 40.70, 36,90, 36,54, 36,32, 36,09, 33,30, 31,61, 29,27, 23,50, 23,39, 21,75, 20,19, 19,52, 18,90, 18,65, 18,50, 16,82, 14,57, 12,20. MS (ESI⁺): m/z 591.15 ([M+Na]⁺). The product was obtained after purification by chromatography on silica gel (MeOH then MeOH/NH₄·OH (10/0.25)). Yield: 10%. Mixture of two diastereomers (β/α) in a ratio (95/05).

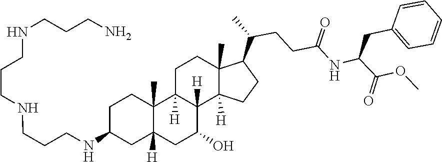

Methyl-L-phenylalaninate 3β-norspermino-chenodeoxycholate 022 (C₄₃H₇₃N₅O₄). NMR ¹H (400 MHz, CD₃OD): δ (ppm)=7.28-7.13 (m, 5H), 4.52-4.49 (m, 1H), 3.80 (s, 1H), 3.52-2.89 (m, 18H), 2.75-0.85 (m, 45H), 0.67 (s, 3H). NMR ¹³C (101 MHz, CD₃OD): δ (ppm)=177.98, 175.80, 139.56, 130.58, 129.49, 129.31, 129.13, 127.33, 68.72, 57.37, 57.00, 51.58, 49.71, 49.50, 45.95, 43.65, 42.98, 41.06, 40.77, 39.26, 37,87, 36,92, 36,56, 36,32, 36,22, 36,16, 35,49, 34,28, 34,06, 33,31, 31,27, 29,30, 29,24, 27,11, 26,47, 25,47, 24,64, 24,58, 23,22, 21,78, 18,91, 16,49, 12,22. MS (ESI⁺): m/z 725.6 ([M+H]⁺). Yield: 7%. Mixture of two diastereomers (β/α) in a ratio (95/05).

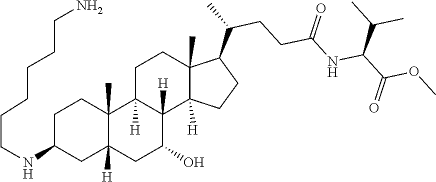

Methyl-L-valinate 3β-(1,6-diamino hexane)-chenodeoxycholate 023 (C₃₆H₆₅N₃O₄). NMR ¹H (400 MHz, CD₃OD): δ (ppm)=4.31-4.17 (m, 1H), 4.07-3.71 (m, 4H), 3.38-2.66 (m, 8H), 2.47-0.83 (m, 49H), 0.71 (s, 3H). NMR ¹³C (101 MHz, CD₃OD): δ (ppm)=177.04, 173.79, 69.02, 59.28, 57.42, 52.40, 51.56, 49.71, 49.50, 49.28, 44.91, 43.69, 41.58, 40.63, 36.98, 36.89, 36.32, 33,65, 33,29, 31,66, 30,11, 29,27, 28,50, 27,81, 27,32, 26,63, 24,57, 23,22, 20,20, 19,76, 19,50, 18,90, 18,63, 18,38, 12,69, 12,20. MS (ESI⁺): m/z 604.5 ([M+H]⁺). The product was obtained after purification by chromatography on silica gel (MeOH then MeOH/NH₄·OH (10/0.5)). Yield: 4%. Mixture of two diastereomers (β/α) in a ratio (95/05).

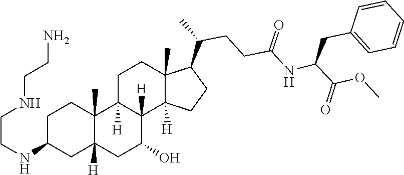

Methyl-L-phenylalanine 3β-(diethylenetriamine)-chenodeoxycholate 024 (C₃₈H₆₂N₄O₄). NMR ¹H (400 MHz, CD₃OD): δ (ppm)=7.29-7.13 (m, 5H), 4.50-4.47 (m, 1H), 3.87-3.81 (m, 1H), 3.71-3.51 (m, 1H), 3.35-2.57 (m, 16H), 2.49-0.89 (m, 35H), 0.68 (s, 3H). NMR ¹³C (101 MHz, CD₃OD): δ (ppm)=178.36, 175.72, 139.67, 130.62, 130.33, 129.49, 129.11, 127.29, 68.76, 59.21, 57.37, 57.28, 51.59, 49.07, 47.18, 46.12, 45.16, 43,65, 43,06, 41,05, 40,76, 40,23, 39,37, 36,93, 36,32, 36,25, 35,52, 34,32, 34,07, 33,91, 33,31, 29,25, 25,26, 24,57, 23,24, 21,76, 18,91, 12,22. MS (ESI⁺): m/z 639.5 ([M+H]⁺). The product was obtained after purification by chromatography on silica gel (MeOH then MeOH/NH₄·OH (10/0.5)). Yield: 7%. Mixture of two diastereomers (β/α) in a ratio (95/05).

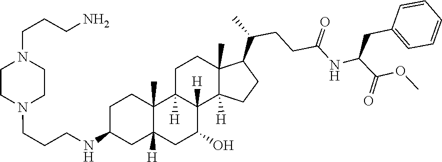

Methyl-L-phenylalaninate 3β-(1,4-bis(3-aminopropyl)piperazine)-chenodeoxycholate 025 (C₄₄H₇₃N₅O₄). NMR ¹H (400 MHz, CD₃OD): δ (ppm)=7.31-7.19 (m, 5H), 4.52 (t, J=4.53 Hz, 1H), 3.81 (s, 1H), 3.42-3.36 (m, 3H), 3.26-2.81 (m, 8H), 2.69-0.87 (m, 52H), 0.68 (s, 3H). NMR ¹³C (101 MHz, CD₃OD): δ (ppm)=176.56, 173.53, 138.55, 130.33, 130.20, 129.48, 127.80, 72.85, 69.04, 57.33, 57.07, 56.95, 56.31, 53.86, 53.83, 51.55, 49.85, 49.71, 49.50, 49.28, 49,07, 43,66, 43,17, 41,05, 40,76, 40,48, 39,11, 38,83, 36,92, 36,55, 36,22, 35,92, 34,07, 33,90, 33,21, 31,36, 29,27, 27,05, 26,95, 24,63, 23,39, 21,78, 18,89, 12,19. MS (ESI⁺): m/z 736.6 ([M+H]⁺). The product was obtained after purification by chromatography on silica gel (MeOH then MeOH/NH₄·OH (10/0.5)). Yield: 5%. Mixture of two diastereomers (β/α) in a ratio (95/05).

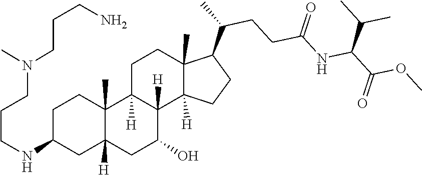

Methyl-L-valinate 3β-(Bis(3-aminopropyl)methylamine)-chenodeoxycholate 026 (C₃₇H₆₈N₄O₄). NMR ¹H (400 MHz, CD₃OD): δ (ppm)=4.31-4.24 (m, 1H), 3.97-3.60 (m, 4H), 3.42-2.79 (m, 10H), 2.62-0.87 (m, 50H), 0.71 (s, 3H). NMR ¹³C (101 MHz, CD₃OD): δ (ppm)=177.03, 173.79, 68.75, 61.22, 59.28, 57.43, 56.43, 54.24, 52.40, 51.63, 49.82, 49.71, 49.50, 49.28, 49.07, 43.70, 43.04, 41,06, 40,77, 36,91, 34,51, 33,69, 33,28, 32,78, 31,66, 29,28, 24,63, 24,58, 23,24, 21,77, 20,22, 19,50, 18,95, 18,89, 18,63, 18,37, 12,21. MS (ESI⁺): m/z 633.5 ([M+H]⁺). The product was obtained after purification by chromatography on silica gel (MeOH then MeOH/NH₄·OH (10/0.5)). Yield: 4%. Mixture of two diastereomers (β/α) in a ratio (95/05).

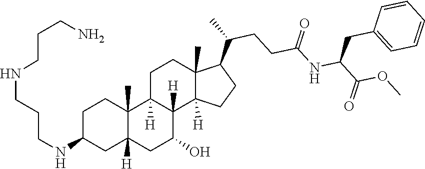

Methyl-L-phenylalaninate 3β-norspermidino-chenodeoxycholate 028 (C₄₀H₆₆N₄O₄). NMR ¹H (400 MHz, CD₃OD): δ (ppm)=7.28-7.12 (m, 5H), 4.52-4.47 (m, 1H), 3.80 (s, 1H), 3.70-3.50 (m, 1H), 3.34 (s, 1H), 3.26-2.80 (m, 12H), 2.71-0.81 (m, 42H), 0.67 (s, 3H). NMR ¹³C (101 MHz, CD₃OD): δ (ppm)=178.38, 175.70, 139.67, 130.62, 130.33, 129.31, 129.11, 127.28, 68.71, 64.40, 59.34, 57.38, 57.26, 54.02, 51.59, 49.85, 49.71, 49.50, 49,28, 49,07, 47,71, 43,65, 43,01, 40,76, 39,36, 38,09, 37,99, 36,93, 36,34, 34,32, 34,07, 30,08, 29,43, 25,28, 24,58, 23,25, 21,77, 18,92, 12,24, 12,21. MS (ESI⁺): m/z 667.5 ([M+H]⁺). The product was obtained after purification by chromatography on silica gel (MeOH then MeOH/NH₄·OH (10/2)). Yield: 10%. Mixture of two diastereomers (β/α) in a ratio (95/05).

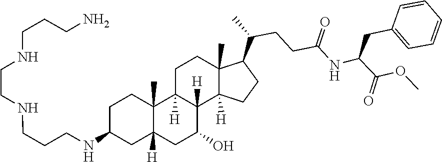

Methyl-L-phenylalaninate 3β-(Bis(3-aminopropyl)ethylenediamine)-chenodeoxycholate 029 (C₄₂H₇₁N₅O₄). NMR ¹H (400 MHz, CD₃OD): δ (ppm)=7.30-7.13 (m, 5H), 4.51-4.48 (m, 1H), 3.80 (s, 1H), 3.69-3.51 (m, 1H), 3.35 (s, 3H), 3.24-2.67 (m, 16H), 2.51-2.41 (m, 1H), 2.23-0.82 (m, 40H), 0.68 (s, 3H). NMR ¹³C (101 MHz, CD₃OD): δ (ppm)=178.24, 175.72, 139.65, 130.62, 129.48, 129.10, 127.85, 127.29, 68.80, 59.44, 57.34, 57.21, 55.16, 52.66, 51.62, 49.85, 49.50, 49.28, 49.07, 47,40, 46,78, 43,66, 43,00, 40,75, 39,34, 36,92, 36,32, 35,46, 34,29, 33,90, 33,32, 32,14, 30,75, 29,25, 26,47, 25,30, 25,17, 24,56, 23,22, 21,77, 18,90, 12,20. MS (ESI⁺): m/z 710.6 ([M+H]⁺). The product was obtained after purification by MeOH then MeOH/NH₄·OH (10/2) silica gel chromatography.) Yield: 8%. Mixture of two diastereomers (β/α) in a ratio (95/05).

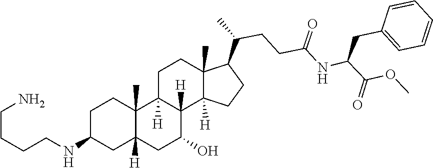

Methyl-L-phenylalaninate 3β-(1,4-diamino butane)-chenodeoxycholate 030 (C₃₈H₆₁N₃O₄). NMR ¹H (400 MHz, CD₃OD): δ (ppm)=7.29-7.13 (m, 5H), 4.52-4.48 (m, 1H), 4.14-3.68 (m, 2H), 3.55-3.35 (m, 2H), 3.24-3.15 (m, 2H), 3.07-2.85 (m, 5H), 2.72-2.38 (m, 1H), 2.23-0.85 (m, 40H), 0.67 (s, 3H). NMR ¹³C (101 MHz, CD₃OD): δ (ppm)=177.99, 175.81, 139.56, 130.57, 130.34, 129.31, 129.14, 127.34, 68.72, 59.25, 57.40, 57.01, 55.16, 54.03, 52.66, 51.58, 44.56, 43,65, 43,01, 41,05, 40,75, 39,60, 39,26, 38,00, 36,92, 36,32, 36,17, 35,50, 34,28, 34,06, 33,80, 33,31, 25,26, 23,92, 23,24, 21,76, 18,92, 12,22. MS (ESI⁺): m/z 624.5 ([M+H]⁺). The product was obtained after purification by chromatography on silica gel (MeOH then MeOH/NH₄·OH (10/1)). Yield: 10%. Mixture of two diastereomers (β/α) in a ratio (95/05).

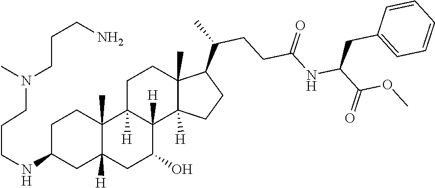

Methyl-L-phenylalaninate 3β-(Bis(3-aminopropyl)methylamine)-chenodeoxycholate 031 (C₄₁H₆₈N₄O₄). NMR ¹H (400 MHz, CD₃OD): δ (ppm)=7.30-7.14 (m, 5H), 4.68-4.50 (m, 1H), 3.85-3.76 (m, 1H), 3.70-3.52 (m, 1H), 3.36 (s, 2H), 3.25-2.67 (m, 7H), 2.53-0.87 (m, 48H), 0.68 (s, 3H). NMR ¹³C (101 MHz, CD₃OD): δ (ppm)=176.69, 173.66, 139.93, 130.33, 130.20, 129.48, 129.31, 127.29, 68.92, 64.40, 59.33, 57.44, 56.96, 56.40, 55.15, 54.01, 52.66, 51.58, 49.50, 49,28, 49,07, 45,78, 43,67, 43,44, 42,12, 41,09, 40,80, 39,40, 38,34, 38,00, 36,94, 36,82, 36,59, 35,83, 34,10, 30,10, 26,70, 24,60, 23,47, 21,77, 12,21. MS (ESI⁺): m/z 682.5 ([M+H]⁺). Yield: 17%. Mixture of two diastereomers (β/α) in a ratio (95/05).

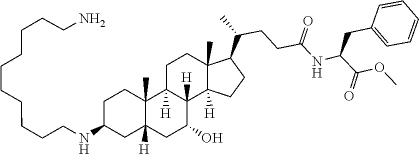

Methyl-L-phenylalaninate 3β-(1,10-diamino decane)-chenodeoxycholate 032 (C₄₄H₇₃N₃O₄). NMR ¹H (400 MHz, CD₃OD): δ (ppm)=7.28-7.12 (m, 5H), 4.49 (q, J₁=4.50 Hz, J₂=4.48 Hz, 1H), 3.86-3.80 (m, 1H), 3.34 (s, 1H), 3.23-3.18 (m, 1H), 3.04-2.59 (m, 7H), 2.38-0.85 (m, 54H), 0.66 (s, 3H). NMR ¹³C (101 MHz, CD₃OD): δ (ppm)=178.28, 175.62, 139.65, 130.64, 129.08, 127.27, 68.77, 59.27, 57.35, 57.20, 51.57, 49.85, 46.27, 43.64, 43.18, 41.67, 41.05, 40.77, 39.41, 36.90, 36.43, 35,66, 35,10, 34,32, 34,08, 33,93, 33,29, 31,31, 31,28, 30,54, 30,51, 30,49, 30,42, 30,36, 29,24, 28,51, 27,95, 27,77, 26,22, 24,58, 23,34, 21,76, 18,92, 12,25. MS (ESI⁺): m/z 708.42 ([M+H]⁺). The product was obtained after purification by chromatography on silica gel (MeOH then MeOH/NH₄·OH (10/0.1)). Yield: 13%. Mixture of two diastereomers (β/α) in a ratio (95/05).

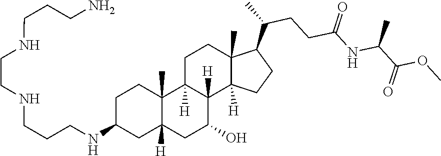

Methyl-L-alaninate 3β-(Bis(3-aminopropyl)ethylenediamine)-chenodeoxycholate 034 (C₃₆H₆₇N₅O₄). NMR ¹H (400 MHz, CD₃OD): δ (ppm)=4.21 (d, J=4.22 Hz, 1H), 3.82-3.71 (m, 2H), 3.55-3.43 (m, 1H), 3.16-2.65 (m, 13H), 2.48-1.77 (m, 18H), 1.67-0.88 (m, 29H), 0.71 (s, 3H). NMR ¹³C (101 MHz, CD₃OD): δ (ppm)=179.88, 175.57, 68.83, 59.40, 57.40, 51.83, 51.61, 49.85, 49.71, 49.50, 49.28, 49.07, 47.77, 43.69, 42.99, 41.01, 40.75, 39,68, 36,99, 36,32, 36,16, 34,34, 34,06, 33,21, 32,14, 30,75, 30,08, 29,27, 26,67, 25,43, 24,56, 23,22, 21,76, 19,49, 18,94, 12,17. MS (ESI⁺): m/z 634.5 ([M+H]⁺). The product was obtained after purification by chromatography on silica gel (MeOH then MeOH/NH₄·OH (10/3)). Yield: 29%. Mixture of two diastereomers (β/α) in a ratio (95/05).

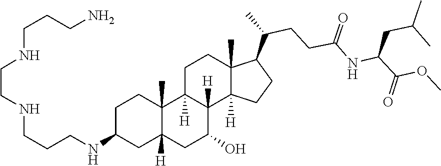

Methyl-L-leucinate 3β-(Bis(3-aminopropyl)ethylenediamine)-chenodeoxycholate 035 (C₃₉H₇₃N₅O₄). NMR ¹H (400 MHz, CD₃OD): δ (ppm)=4.37 (s, 1H), 3.82 (s, 1H), 3.72 (s, 2H), 2.97-2.66 (m, 13H), 2.51-2.17 (m, 4H), 2.05-0.90 (m, 49H), 0.72 (s, 3H). NMR ¹³C (101 MHz, CD₃OD): δ (ppm)=176.95, 173.83, 68.83, 59.23, 58.24, 57.62, 52.35, 51.65, 49.82, 49.71, 49.50, 49.28, 48.18, 46.13, 45.21, 43.71, 43.23, 41.10, 40,77, 40,28, 38,22, 36,91, 36,47, 35,70, 34,13, 33,74, 33,29, 30,76, 30,08, 29,28, 29,27, 26,42, 24,57, 23,33, 21,76, 18,89, 15,98, 12,20, 11,61. MS (ESI⁺): m/z 676.86 ([M+H]⁺). The product was obtained after purification by chromatography on silica gel (MeOH then CH₂Cl₂/MeOH/NH₄·OH (7/3/0.75)). Yield: 2%. Mixture of two diastereomers (β/α) in a ratio (95/05).

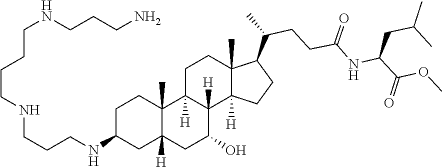

Methyl-L-leucinate 3β-spermino-chenodeoxycholate 036 (C₄₁H₈₈N₅O₄). NMR ¹H (400 MHz, CD₃OD): δ (ppm)=4.37-4.26 (m, 1H), 3.81 (s, 1H), 3.72 (s, 2H), 3.40-3.18 (m, 4H), 2.99-1.07 (m, 52H), 1.00-0.90 (m, 14H), 0.71 (s, 3H). NMR ¹³C (101 MHz, CD₃OD): δ (ppm)=176.93, 173.81, 68.93, 60.85, 59.28, 58.23, 57.56, 52.35, 51.60, 50.37, 49.71, 49.50, 49.28, 49.07, 45.54, 43.71, 43.48, 41.12, 40.82, 40,53, 39,53, 38,23, 36,61, 34,52, 34,10, 33,71, 33,45, 33,29, 29,29, 28,12, 26,42, 26,14, 24,61, 23,49, 22,17, 21,78, 18,92, 16,46, 16,00, 12,22, 11,63. MS (ESI⁺): m/z 704.6 ([M+H]⁺). Yield: 4%. Mixture of two diastereomers (β/α) in a ratio (95/05).

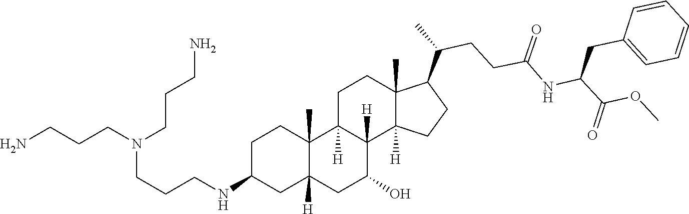

Methyl-L-phenylalaninate 3β-(Tris(3-aminopropyl)amine)-chenodeoxycholate 037 (C₄₃H₇₃N₅O₄). NMR ¹H (400 MHz, CD₃OD): δ (ppm)=7.28-7.14 (m, 5H), 4.48-4.45 (m, 1H), 3.80 (s, 1H), 3.25-3.07 (m, 2H), 2.99-2.82 (m, 7H), 2.72-2.39 (m, 9H), 2.24-1.06 (m, 38H), 1.01-0.88 (m, 7H), 0.67 (s, 3H). NMR ¹³C (101 MHz, CD₃OD): δ (ppm)=178.46, 175.75, 139.68, 130.58, 130.33, 129.31, 129.15, 127.33, 68.77, 64.40, 59.29, 57.37, 54.03, 52.52, 52.29, 51.63, 44.72, 43.66, 43.07, 41.06, 40,74, 40,14, 39,91, 39,40, 37,99, 36,95, 36,38, 36,27, 35,58, 34,32, 34,11, 33,33, 30,74, 29,26, 27,13, 27,02, 25,88, 25,15, 24,56, 23,27, 21,76, 18,95, 12,24. MS (ESI⁺): m/z 724.16 ([M+H]⁺). The product was obtained after purification by chromatography on silica gel (MeOH then CH₂C₂/MeOH/NH₄·OH (7/3/1.5)). Yield: 7%. Mixture of two diastereomers (β/α) in a ratio (95/05).

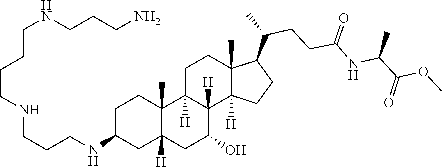

Methyl-L-alaninate 3β-spermino-chenodeoxycholate 038 (C₃₈H₇₁N₅O₄). NMR ¹H (400 MHz, CD₃OD): δ (ppm)=4.27-4.18 (m, 1H), 3.94-3.79 (m, 1H), 3.51-3.40 (m, 1H), 3.35 (s, 2H), 3.27-3.02 (m, 2H), 2.92-2.57 (m, 13H), 2.43-0.90 (m, 48H), 0.70 (s, 3H). NMR ¹³C (101 MHz, CD₃OD): δ (ppm)=179.94, 175.53, 68.84, 66.21, 59.22, 57.40, 51.87, 51.62, 50.01, 49.91, 49.85, 48.25, 48.00, 43.70, 43.33, 41.10, 40.80, 40.32, 40,29, 37,79, 36,99, 36,69, 36,53, 34,30, 34,10, 33,83, 33,35, 33,23, 31,36, 29,30, 27,72, 24,61, 23,41, 21,77, 19,49, 18,98, 17,13, 12,19. MS (ESI⁺): m/z 663.6 ([M+H]⁺).

The product was obtained after purification by chromatography on silica gel (MeOH then CH₂C₂/MeOH/NH₄·OH (7/3/1.5)). Yield: 4%. Mixture of two diastereomers (β/α) in a ratio (95/05).

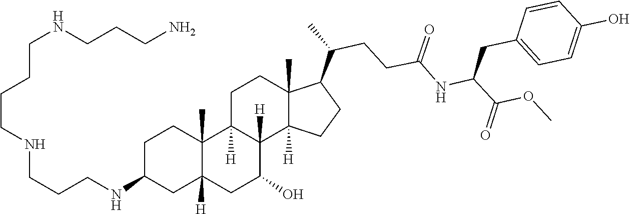

Methyl-L-tyrosinate 3β-spermino-chenodeoxycholate 039 (C₄₄H₇₅N₅O₅). NMR ¹H (400 MHz, CD₃OD): δ (ppm)=7.05-6.99 (m, 2H), 6.71-6.65 (m, 2H), 3.80 (s, 1H), 3.68 (s, 2H), 3.35 (s, 1H), 3.23-3.02 (m, 2H), 2.90-2.47 (m, 15H), 2.27-0.92 (m, 47H), 0.68 (s, 3H). NMR ¹³C (101 MHz, CD₃OD): δ (ppm)=176.69, 173.84, 157.57, 131.17, 128.75, 116.29, 68.93, 59.26, 57.46, 57.38, 55.44, 52.61, 51.58, 50.33, 50.19, 49.85, 49.50, 49.28, 49.07, 48.16, 45.52, 43,68, 43,45, 41,08, 40,79, 40,50, 37,61, 36,84, 36,71, 36,59, 35,85, 34,09, 33,76, 33,20, 32,47, 29,25, 28,97, 28,05, 27,68, 24,61, 23,48, 21,77, 18,86, 12,21. MS (ESI⁺): m/z 754.6 ([M+H]⁺). The product was obtained after purification by chromatography on silica gel (MeOH then CH₂C₂/MeOH/NH₄·OH (7/3/1.5)). Yield: 7%. Mixture of two diastereomers (β/α) in a ratio (95/05).

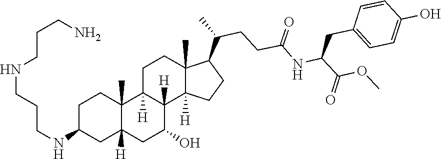

Methyl-L-tyrosinate 3β-norspermidino-chenodeoxycholate 040 (C₄₀H₆₆N₄O₅). NMR ¹H (400 MHz, CD₃OD): δ (ppm)=7.06-6.97 (m, 2H), 6.75-6.65 (m, 2H), 4.61-4.57 (m, 1H), 3.81 (s, 1H), 3.69 (s, 2H), 3.52-3,35 (m, 1H), 3.25-2.96 (m, 3H), 2.91-1.71 (m, 25H), 1.57-1.09 (m, 18H), 1.04-0.87 (m, 8H), 0.69 (s, 3H). NMR ¹³C (101 MHz, CD₃OD): δ (ppm)=176.71, 173.85, 157.48, 131.58, 131.18, 128.82, 116.25, 115.88, 68.95, 64.38, 63.80, 61.09, 57.48, 57.35, 55.43, 53.69, 52.61, 51.58, 49,71, 49,50, 49,28, 49,07, 43,68, 41,07, 41,06, 40,78, 39,64, 37,61, 36,83, 36,56, 34,09, 33,76, 33,19, 30,75, 29,24, 24,60, 23,43, 21,76, 18,85, 12,20. MS (ESI⁺): m/z 683.5 ([M+H]⁺). The product was obtained after purification by chromatography on silica gel (MeOH then CH₂C₂/MeOH/NH₄·OH (7/3/0.75)). Yield: 4%. Mixture of two diastereomers (β/α) in a ratio (90/10).

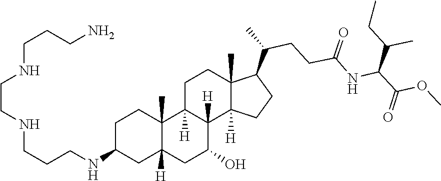

Methyl-L-isoleucinate 3β-(Bis(3-aminopropyl)ethylenediamine)-chenodeoxycholate 041 (C₃₉H₇₃N₅O₄). NMR ¹H (400 MHz, CD₃OD): δ (ppm)=4.36 (d, J=4.35 Hz, 1H), 3.78 (s, 1H), 3.70 (s, 2H), 3.34 (s, 1H), 3.26-3.14 (m, 1H), 2.91-2.61 (m, 12H), 2.49-2.15 (m, 5H), 2.03-1.82 (m, 9H), 1.77-1.65 (m, 8H), 1.58-1.09 (m, 17H), 1.04-0.88 (m, 13H), 0.69 (s, 3H). NMR ¹³C (101 MHz, CD₃OD): δ (ppm)=176.62, 173.55, 68.66, 59.07, 57.98, 57.26, 52.14, 51.34, 49.64, 49.39, 49.33, 48.05, 45.22, 43.46, 43.26, 40.85, 40.58, 40.27, 37,98, 36,65, 36,47, 36,38, 35,67, 33,84, 33,45, 33,06, 32,59, 30,76, 29,35, 29,06, 27,57, 26,19, 24,40, 23,33, 21,56, 18,72, 15,81, 12,04, 11,45. MS (ESI⁺): m/z 676.6 ([M+H]⁺). Yield: 21%. Mixture of two diastereomers (β/α) in a ratio (95/05).

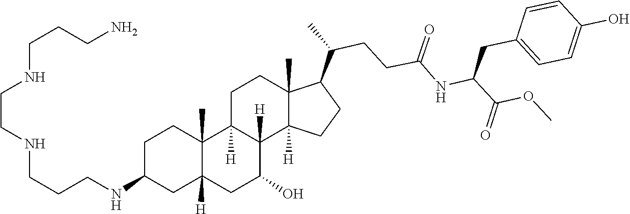

Methyl-L-tyrosinate 3β-(Bis(3-aminopropyl)ethylenediamine)-chenodeoxycholate 042 (C₄₂H₇₁N₅O₅). NMR ¹H (400 MHz, CD₃OD): δ (ppm)=7.06-6.99 (m, 2H), 6.71-6.64 (m, 2H), 3.79 (s, 1H), 3.68 (s, 1H), 3.50-3.33 (m, 2H), 3.24-0.90 (m, 60H), 0.67 (s, 3H). NMR ¹³C (101 MHz, CD₃OD): δ (ppm)=176.67, 173.84, 157.50, 131.17, 128.78, 116.27, 68.85, 59.24, 57.47, 55.43, 52.62, 51.59, 49.85, 48.21, 47.56, 45.59, 45.30, 43.66, 43.30, 41.07, 40,77, 40,36, 37,60, 36,93, 36,83, 36,63, 36,51, 36,07, 35,76, 34,08, 33,76, 33,18, 31,68, 29,93, 29,24, 28,46, 27,00, 24,59, 23,40, 21,76, 18,86, 12,21. MS (ESI⁺): m/z 726.6 ([M+H]⁺). Yield: 9%. Mixture of two diastereomers (β/α) in a ratio (95/05).

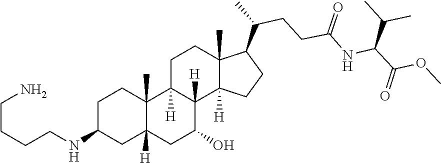

Methyl-L-valinate 3β-(1,4-diamino butane)-chenodeoxycholate 043 (C₃₄H₆₁N₃O₄). NMR¹ H (400 MHz, CD₃ OD): δ (ppm)=4.32 (d, J=4.30 Hz, 1H), 3.85-3.62 (m, 5H), 3.36 (s, 2H), 2.91-0.90 (m, 50H), 0.71 (s, 3H). NMR ¹³C (101 MHz, CD₃OD): δ (ppm)=177.05, 173.79, 68.96, 59.28, 57.51, 52.40, 51.57, 49.85, 46.81, 43.70, 43.47, 42.00, 41.08, 40.80, 36.94, 36.91, 36,88, 36,61, 35,85, 34,08, 33,68, 33,29, 31,66, 30,91, 29,27, 27,72, 27,56, 24,60, 23,50, 21,77, 19,50, 18,91, 18,63, 12,21. MS (ESI⁺): m/z 576.5 ([M+H]⁺). The product was obtained after purification by chromatography on silica gel (MeOH then CH₂C₂/MeOH/NH₄·OH (7/3/0.6)). Yield: 3%. Mixture of two diastereomers (β/α) in a ratio (95/05).

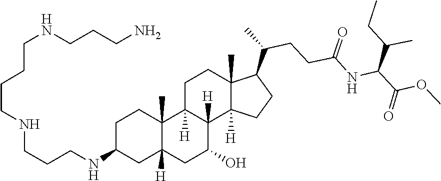

Methyl-L-isoleucinate 3β-spermino-chenodeoxycholate 044 (C₄₁H₇₇N₅O₄). NMR ¹H (400 MHz, CD₃OD): δ (ppm)=4.36 (d, J=4.35 Hz, 1H), 3.79 (s, 1H), 3.71 (s, 2H), 3.35 (s, 1H), 3.24-3.16 (m, 1H), 2.92-2.61 (m, 12H), 2.52-2.45 (m, 1H), 2.35-2.15 (m, 3H), 2.03-1.66 (m, 25H), 1.58-1.05 (m, 21H), 1.02-0.90 (m, 13H), 0.70 (s, 3H). NMR ¹³C (101 MHz, CD₃OD): δ (ppm)=176.91, 173.80, 68.91, 59.27, 58.22, 57.53, 52.35, 51.59, 50.35, 50.22, 49.85, 49.50, 49.28, 49.07, 48.68, 48.16, 45.51, 43.70, 43.47, 41,10, 40,81, 40,50, 38,22, 36,96, 36,88, 36,60, 35,86, 34,09, 33,69, 33,29, 32,52, 29,29, 28,06, 26,41, 24,61, 23,50, 21,78, 18,92, 16,00, 12,22, 11,63. MS (ESI⁺): m/z 704.6 ([M+H]⁺). The product was obtained after purification by chromatography on silica gel (MeOH then CH₂Cl₂/MeOH/NH₄·OH (7/3/1.5)). Yield: 14%. Mixture of two diastereomers (β/α) in a ratio (95/05).

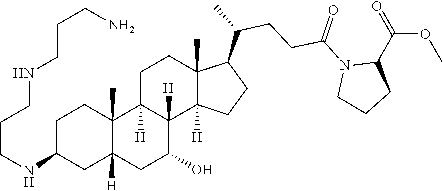

Methyl-L-prolinate 3β-norspermidino-chenodeoxycholate 045 (C₃₆H₆₄N₄O₄). NMR ¹H (400 MHz, CD₃OD): δ (ppm)=4.43-4.14 (m, 1H), 3.86-3.77 (m, 2H), 3.71 (s, 2H), 3.68-3.34 (m, 4H), 2.95-2.66 (m, 7H), 2.59-0.93 (m, 45H), 0.72 (s, 3H). NMR ¹³C (101 MHz, CD₃OD): δ (ppm)=175.06, 174.52, 68.90, 60.24, 59.24, 57.47, 52.68, 51.60, 51.54, 49.28, 48.72, 48.50, 48.06, 45.47, 43.69, 43.39, 41.08, 40,80, 40,42, 36,89, 36,75, 36,56, 35,79, 34,09, 32,32, 32,20, 32,10, 30,26, 29,27, 28,69, 25,74, 24,61, 23,45, 21,76, 19,02, 12,20. MS (ESI⁺): m/z 617.5 ([M+H]⁺). The product was obtained after purification by chromatography on silica gel (MeOH then CH₂Cl₂/MeOH/NH₄·OH (7/3/0.7)). Yield: 10%. Mixture of two diastereomers (β/α) in a ratio (95/05).

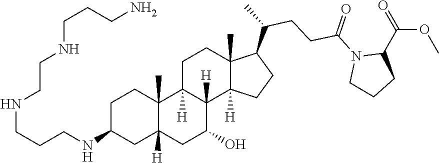

Methyl-L-prolinate 3β-(Bis(3-aminopropyl)ethylenediamine)-chenodeoxycholate 046 (C₃₈H₆₉N₅O₄). NMR ¹H (400 MHz, CD₃OD): δ (ppm)=4.42-4.13 (m, 1H), 3.80-3.76 (m, 1H), 3.70 (s, 2H), 3.67-3.33 (m, 4H), 3.19-3.06 (m, 1H), 2.96-2.63 (m, 11H), 2.56-0.92 (m, 46H), 0.71 (s, 3H). NMR ¹³C (101 MHz, CD₃OD): δ (ppm)=175.04, 174.51, 68.88, 62.22, 60.23, 59.27, 57.45, 52.69, 51.57, 49.55, 49.48, 49.28, 48.67, 48.49, 48.25, 45.38, 43.68, 43.42, 41,06, 40,79, 40,44, 36,88, 36,67, 36,57, 34,06, 32,48, 32,30, 32,09, 30,26, 29,27, 29,19, 27,51, 25,73, 24,60, 23,48, 21,76, 19,03, 12,22. MS (ESI⁺): m/z 661.6 ([M+H]⁺). Yield: 21%. Mixture of two diastereomers (β/α) in a ratio (95/05).

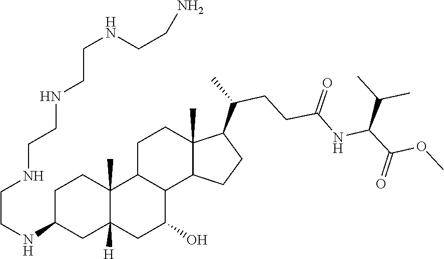 [SMILES: COC(=O)[C@@H](NC(=O)CC[C@@H](C)[C@H]1CCC2C3C(CC[C@@]21C)[C@@]1(C)CC[C@H](NCCNCCNCCNCCN)C[C@H]1C[C@H]3O)C(C)C]

Methyl-L-valinate 3β-(tetraethylenetetramine)-chenodeoxycholate 049 (C₃₈H₇₂N₆O₄). NMR ¹H (400 MHz, CD₃OD): δ (ppm)=6.93 (m, 1H), 4.47-4.45 (m, 1H), 3.93-3.82 (m, 2H), 3.51-3.47 (m, 3H), 3.05-2.99 (m, 2H), 2.97-2.86 (m, 4H), 2.87-2.63 (m, 6H), 2.63-2.61 (m, 10H), 2.19-1.94 (m, 3H), 1.91-1.09 (m, 23H), 1.02-0.82 (m, 14H), 0.68 (s, 3H). NMR ¹³C (101 MHz, CD₃OD): δ (ppm)=174.40, 173.62, 68.85, 56.12, 55.75, 54.74, 54.51, 51.81, 50.50, 48.39, 46.94, 46.83, 46.73, 45.47, 45.37, 42.62, 41.96, 40.96, 39.74, 39.50, 37.80, 36.36, 36.25, 36.15, 36.06, 35.39, 33.56, 33.10, 31.28, 28.50, 28.22, 23.58, 22.90, 18.90, 17.82, 12.15, 11.69. MS (ESI⁺): m/z 678.89 ([M+H]⁺). The product was obtained after purification by chromatography on silica gel (MeOH then MeOH/NH₄·OH (10/3)). Yield: 19%. Mixture of two diastereomers (β/α) in a ratio (95/05).

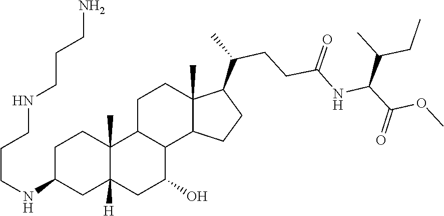 [SMILES: CCC(C)[C@H](NC(=O)CC[C@@H](C)[C@H]1CCC2C3C(CC[C@@]21C)[C@@]1(C)CC[C@H](NCCCNCCCN)C[C@H]1C[C@H]3O)C(=O)OC]

Methyl-L-isoleucinate 3β-norspermidino-chenodeoxycholate 050 (C₃₇H₆₈N₄O₄). NMR ¹H (400 MHz, CD₃OD): δ (ppm)=6.94 (m, 1H), 3.93-3.83 (m, 2H), 3.76-3.70 (m, 1H), 3.51-3.47 (m, 3H), 2.99-2.87 (m, 2H), 2.81-2.65 (m, 2H), 2.64-2.56 (m, 2H), 2.55-2.49 (m, 4H), 2.29-2.17 (m, 2H), 2.13-1.93 (m, 2H), 1.91-1.08 (m, 30H), 1.02-0.82 (m, 14H), 0.70-0.68 (m, 3H). NMR ¹³C (101 MHz, CD₃OD): δ (ppm)=174.41, 172.73, 68.85, 57.24, 56.12, 55.75, 52.00, 50.50, 46.94, 46.00, 45.97, 42.62, 40.96, 39.74, 39.50, 39.21, 37.80, 36.54, 36.36, 36.25, 36.15, 36.06, 35.39, 33.56, 33.10, 28.50, 28.21, 27.14, 25.00, 24.25, 23.58, 22.90, 18.90, 15.72, 12.15, 11.99, 11.68. MS (ESI⁺): m/z 634.05 ([M+H]⁺). The product was obtained after purification by chromatography on silica gel (MeOH then MeOH/NH₄·OH (10/3)). Yield: 31%. Mixture of two diastereomers (β/α) in a ratio (95/05).

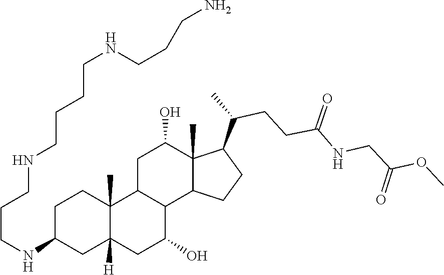 [SMILES: COC(=O)CNC(=O)CC[C@@H](C)[C@H]1CCC2C3C(C[C@H](O)[C@@]21C)[C@@]1(C)CC[C@H](NCCCNCCCCNCCCN)C[C@H]1C[C@H]3O]

Methyl-glycinate 3β-spermino cholate 051 (C₃₇H₆₉N₅O₅). NMR ¹H (400 MHz, CD₃OD): δ (ppm)=7.06-7.03 (m, 1H), 4.12-3.71 (m, 8H), 2.99-2.91 (m, 7H), 2.81-2.49 (m, 8H), 2.29-1.20 (m, 31H), 1.17-0.81 (m, 10H), 0.73-0.69 (m, 3H). NMR ¹³C (101 MHz, CD₃OD): δ (ppm)=173.69, 173.36, 73.55, 69.98, 55.75, 52.58, 47.98, 47.60, 47.49, 46.94, 46.42, 45.97, 44.61, 43.56, 43.34, 40.96, 39.21, 39.03, 37.80, 36.36, 36.06, 35.34, 35.12, 34.33, 33.50, 31.32, 29.34, 28.50, 27.38, 27.14, 25.38, 25.28, 24.25, 24.02, 21.43, 18.20, 12.71. MS (ESI⁺): m/z 664.59 ([M+H]⁺). The product was obtained after purification by chromatography on silica gel (MeOH then MeOH/NH₄·OH (10/3)).

Yield: 27%. Mixture of two diastereomers (β/α) in a ratio (95/05).

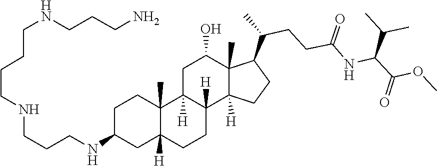

Methyl-L-valinate 3β-spermino deoxycholate 052 (C₄₀H₇₅N₅O₄). NMR ¹H (250 MHz, CD₃OD): δ (ppm)=5.11-4.63 (m, 2H), 4.28 (m, 1H), 3.92 (s, 1H), 3.69 (m, 3H), 3.39-3.31 (m, 1H), 2.88-2.46 (m, 15H), 2.34-1.23 (m, 34H), 1.14-0.82 (m, 15H), 0.67 (s, 3H). NMR ¹³C (63 MHz, CD₃OD): δ (ppm)=177.02, 173.74, 74.02, 59.22, 58.90, 57.17, 52.41, 50.71, 50.42, 49.16, 48.18, 47.61, 45.62, 43.89, 40.54, 40.15, 38.21, 37.42, 36.87, 35,82, 35,73, 34,79, 34,19, 33,66, 33,29, 32,84, 31,65, 29,82, 28,70, 28,46, 28,10, 27,49, 27,24, 24,90, 24,06, 23,92, 19,55, 18,66, 17,70, 13,24. MS (ESI⁺): m/z 690.5892 ([M+H]⁺). Yield: 71%. Mixture of two diastereomers (β/α) in a ratio (95/05).

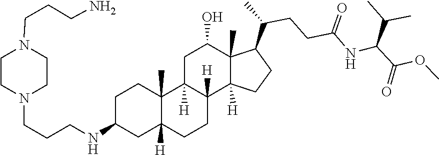

Methyl-L-valinate 3β-(1,4-bis(3-aminopropyl)piperazine)-deoxycholate 053 (C₄₀H₇₃N₅O₄). NMR ¹H (400 MHz, CD₃OD): δ (ppm)=4.35 (d, J=3.34 Hz, 1H), 4.02 (s, 1H), 3.76 (s, 2H), 3.40 (s, 1H), 3.29 (s, 2H), 3.12 (s, 1H), 3.00-2.87 (m, 5H), 2.70-2.13 (m, 16H), 2.00-1.33 (m, 26H), 1.23-0.93 (m, 15H), 0.76 (s, 3H). NMR ¹³C (101 MHz, CD₃OD): δ (ppm)=177.31, 174.04, 74.17, 59.52, 59.09, 57.73, 54.29, 54.04, 52.60, 50.06, 49.93, 49.71, 49.50, 49.28, 49.07, 48.88, 47.87, 46.07, 43.80, 41,52, 40,85, 37,61, 37,14, 36,59, 36,39, 35,75, 35,12, 34,04, 33,51, 31,88, 28,92, 28,40, 26,88, 25,03, 19,69, 18,85, 17,90, 17,90, 17,89, 13,38. MS (ESI⁺): m/z 688.6 ([M+H]⁺). Yield: 8%. Mixture of two diastereomers (β/α) in a ratio (95/05).

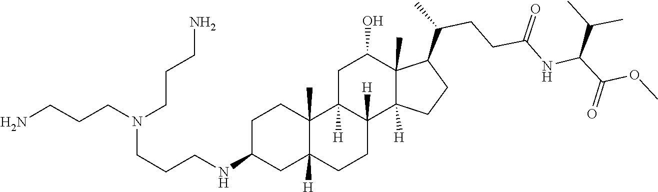

Methyl-L-valinate 3β-(Tris(3-aminopropyl)amine)-deoxycholate 054 (C₃₉H₇₃N₅O₄). NMR ¹H (400 MHz, CD₃OD): δ (ppm)=4.35 (d, J=4.35 Hz, 1H), 4.02 (s, 1H), 3.75 (s, 2H), 3.39 (s, 3H), 3.11-2.12 (m, 19H), 1.94-1.29 (m, 29H), 1.20-0.90 (m, 15H), 0.76 (s, 3H). NMR ¹³C (101 MHz, CD₃OD): δ (ppm)=177.09, 173.82, 73.98, 59.30, 54.88, 52.80, 52.38, 49.85, 49.71, 49.50, 49.28, 49.07, 48.85, 48.69, 48.18, 47.65, 43.71, 42.78, 40,51, 37,40, 36,91, 35,61, 34,90, 33,80, 33,29, 31,67, 29,85, 28,69, 27,44, 27,24, 25,71, 24,84, 23,70, 19,48, 18,64, 17,69, 17,27, 17,08, 13,19. MS (ESI⁺): m/z 676.6 ([M+H]⁺). Yield: 7%. Mixture of two diastereomers (β/α) in a ratio (95/05).

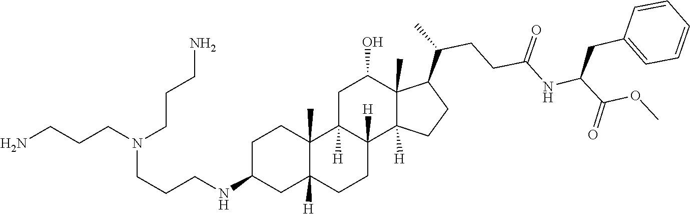

Methyl-L-phenylalaninate 3β-(Tris(3-aminopropyl)amine)-deoxycholate 055 (C₄₃H₇₃N₅O₄). NMR ¹H (400 MHz, CD₃OD): δ (ppm)=7.45-7.19 (m, 5H), 5.25 (s, 1H), 3.96 (s, 1H), 3.35 (s, 6H), 3.08-3.01 (m, 6H), 2.83-0.89 (m, 49H), 0.69-0.66 (m, 3H). NMR ¹³C (101 MHz, CD₃OD): δ (ppm)=176.84, 175.34, 141.83, 129.23, 129.20, 128.43, 128.40, 128.12, 74.06, 60.63, 60.56, 58.97, 52.73, 52.37, 49.85, 49.71, 49.50, 49.28, 47.66, 44.11, 43,46, 39,64, 37,32, 36,87, 36,13, 35,43, 34,71, 34,32, 34,26, 33,24, 30,85, 30,68, 29,73, 28,72, 28,68, 28,02, 27,33, 25,45, 25,36, 23,47, 17,78, 17,73, 13,16. MS (ESI⁺): m/z 724.6 ([M+H]⁺). The product was obtained after purification by chromatography on silica gel (MeOH then MeOH/NH₄·OH (10/2)). Yield: 12%. Mixture of two diastereomers (β/α) in a ratio (95/05).

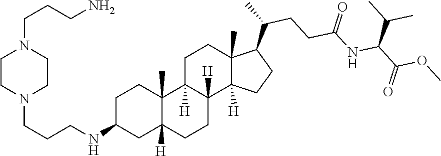

Methyl-L-valinate 3β-(1,4-bis(3-aminopropyl)piperazine)-lithocholate 056 (C₄₀H₇₅N₅O₃). NMR ¹H (400 MHz, CD₃OD): δ (ppm)=4.30 (d, J=4.31 Hz, 1H), 4.23 (t, J=4.23 Hz, 1H), 3.71 (s, 2H), 3.47-2.71 (m, 17H), 2.47-1.80 (m, 16H), 1.62-0.86 (m, 33H), 0.71 (s, 3H). NMR ¹³C (101 MHz, CD₃OD): δ (ppm)=177.02, 173.79, 59.29, 57.92, 57.71, 57.56, 52.40, 49.83, 49.71, 49.49, 49.28, 49.07, 43.95, 43.31, 41.81, 41.52, 41.32, 38.14, 37.13, 36,91, 36,84, 36,08, 35,78, 33,69, 33,28, 32,52, 31,66, 29,27, 27,93, 27,49, 25,23, 23,73, 21,93, 20,15, 19,51, 18,93, 18,88, 18,64, 18,44, 12,50. MS (ESI⁺): m/z 672.6 ([M+H]⁺). The product was obtained after purification by chromatography on silica gel (MeOH then MeOH/NH₄·OH (10/0.75)). Yield: 6%. Mixture of two diastereomers (β/α) in a ratio (95/05).

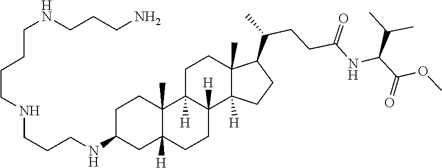

Methyl-L-valinate 3β-spermino-lithocholate 057 (C₄₀H₇₅N₅O₃). NMR ¹H (400 MHz, CD₃OD): δ (ppm)=4.23-4.21 (m, 1H), 2.94-2.68 (m, 15H), 2.37-1.11 (m, 43H), 1.04-0.89 (m, 13H), 0.71 (s, 3H). NMR ¹³C (101 MHz, CD₃OD): δ (ppm)=178.68, 175.99, 61.34, 58.86, 58.05, 57.53, 50.04, 49.89, 49.50, 49.28, 49.07, 48.49, 47.98, 45.47, 43.94, 43.61, 41.92, 41.57, 40.18, 37,17, 36,92, 36,61, 35,98, 34,40, 33,46, 33,15, 32,70, 30,62, 29,30, 28,22, 28,12, 27,75, 27,61, 25,25, 23,96, 21,95, 20,24, 18,92, 18,43, 12,50. MS (ESI⁺): m/z 674.6 ([M+H]⁺). Yield: 4%. Mixture of two diastereomers (β/α) in a ratio (95/05).

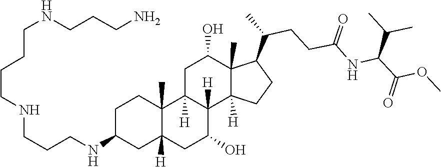

Methyl-L-valinate 3β-spermino-cholate 058 (C₄₀H₇₅N₅O₅). NMR ¹H (250 MHz, CD₃OD): δ (ppm)=4.79 (m, 11H), 4.35-3.26 (m, 6H), 2.98-2.66 (m, 13H), 2.40-1.14 (m, 33H), 1.09-1.05 (m, 3H), 1.01-0.94 (m, 6H), 0.75 (s, 3H). NMR ¹³C (63 MHz, CD₃OD): δ (ppm)=177.01, 173.76, 73.87, 68.84, 59.27, 59.21, 52.39, 50.13, 48.17, 48.07, 47.54, 45.46, 43.43, 43.01, 41.06, 40.44, 36.93, 36.72, 36,57, 36,21, 33,77, 33,30, 31,95, 31,85, 31,65, 29,59, 28,73, 27,93, 27,89, 27,26, 24,20, 23,26, 20,26, 19,51, 18,67, 18,45, 17,76, 13,04. MS (ESI⁺): m/z 706.6 ([M+H]⁺). The product was obtained after purification by chromatography on silica gel (MeOH then CH₂C₂/MeOH/NH₄·OH (7/3/2)). Yield: 45%. Mixture of two diastereomers (β/α) in a ratio (95/05).

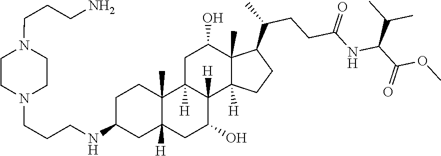

Methyl-L-valinate 3β-(1,4-bis(3-aminopropyl)piperazine)-cholate 059 (C₄₀H₇₃N₅O₅). NMR ¹H (250 MHz, CD₃OD): δ (ppm)=4.71 (m, 2H), 4.10-3.31 (m, 6H), 2.94-2.07 (m, 30H), 2.02-1.14 (m, 29H), 1.11-1.06 (m, 3H), 0.73 (s, 3H). NMR ¹³C (63 MHz, CD₃OD): δ (ppm)=177.07, 173.80, 73.89, 68.86, 59.27, 57.28, 53.93, 53.90, 52.39, 49.78, 49.23, 48.18, 47.55, 45.91, 43.46, 43.03, 40.93, 37.65, 36.94, 36,74, 36,22, 34,23, 33,74, 33,30, 32,78, 31,67, 29,78, 29,61, 28,71, 27,99, 27,99, 27,29, 26,09, 24,21, 23,27, 19,50, 18,65, 17,74, 16,39, 13,04. MS (ESI⁺): m/z 704.6 ([M+H]⁺). Yield: 28%. Mixture of two diastereomers (β/α) in a ratio (95/05).

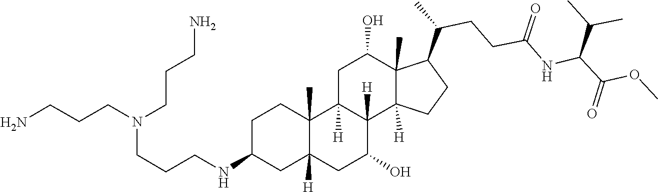

Methyl-L-valinate 3β-(Tris(3-aminopropyl)amine)-cholate 060 (C₃₉H₇₃N₅O₅). NMR ¹H (250 MHz, CD₃OD): δ (ppm)=4.95 (s, 1H), 4.88 (s, 11H), 4.79 (s, 1H), 4.27 (d, J=4.27 Hz, 1H), 3.96-3.51 (m, 5H), 3.43-3.23 (m, 3H), 3.05-2.47 (m, 13H), 2.32-1.07 (m, 35H), 0.67 (s, 3H). NMR ¹³C (63 MHz, CD₃OD): δ (ppm)=177.11, 173.79, 73.85, 68.81, 59.28, 59.18, 52.62, 52.41, 49.95, 49.85, 48.79, 48.47, 47.50, 47.46, 45.48, 43.19, 42.99, 41.05, 40,65, 40,48, 37,00, 36,11, 35,89, 33,78, 33,69, 33,31, 31,65, 31,14, 29,58, 28,91, 27,98, 25,68, 25,24, 24,19, 23,16, 19,51, 18,64, 17,71, 13,02. MS (ESI⁺): m/z 692.6 ([M+H]⁺). Yield: 12%. Mixture of two diastereomers (β/α) in a ratio (95/05).

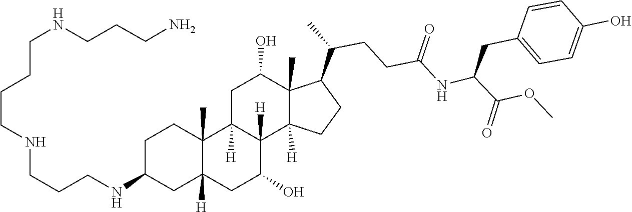

Methyl-L-tyrosinate 3β-spermino-cholate 061 (C₄₄H₇₅N₅O₆). NMR ¹H (250 MHz, CD₃OD): δ (ppm)=6.93 (m, 2H), 6.61 (m, 2H), 4.71 (s, 1H), 4.47 (q, J₁=4.51 Hz, J₂=4.48 Hz, 1H), 3.87-3.43 (m, 6H), 3.31-3.14 (m, 3H), 2.88-2.70 (m, 11H), 2.39-1.19 (m, 46H), 0.62 (s, 3H). NMR ¹³C (63 MHz, CD₃OD): δ (ppm)=176.72, 173.83, 157.42, 131.18, 128.81, 116.26, 73.83, 68.84, 59.31, 55.45, 52.65, 51.66, 50.60, 50.12, 49.84, 49.50, 48.09, 47.49, 45.86, 43.03, 40.95, 39,00, 37,63, 36,24, 36,02, 36,02, 34,41, 33,18, 29,54, 29,47, 28,80, 27,87, 27,10, 25,86, 25,39, 24,19, 23,00, 22,29, 21,83, 19,80, 17,77, 17,72, 15,88, 12,98. MS (ESI⁺): m/z 770.6 ([M+H]⁺). Yield: 25%. Mixture of two diastereomers (β/α) in a ratio (95/05).

### D. Preparation of the Different Salts

### D.1 from Hydrochloric Acid

The compounds 001-061 obtained as described above were all prepared as hydrochloride salts for biological testing, according to the following general procedure described below for the compound 007.

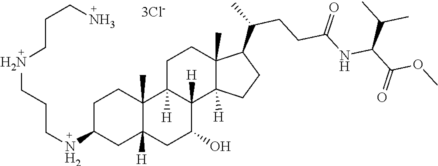

Methyl-L-valinate 3β-norspermidino-chenodeoxycholate 007 as hydrochloride salt. In a flask, 50 mg (0.0808 mmol) of 007 (007-a1, I/α: 96/04) is dissolved in a minimal amount of methanol. Under vigorous stirring, 0.24 mL (0.242 mmol) of a 2 N hydrochloric acid solution is added. Stirring is continued for a few minutes and the solution is evaporated. The solid obtained is taken up in diethyl ether and filtered. The derivative S007 (007.3 HCl) (β/α, 96/04) is obtained as a yellow solid in quantitative yield.

Others hydrochloric salts S019 (β/α: 94/06) and S021 (β/α: 90/10) were prepared from the compounds 007-a2 (β/α: 94/06) and 007-a3 (β/α: 90/10) respectively, following the above-described method.

A similar procedure may be applied to other inorganic acids or organic acids such as lactic acid, citric acid, malic acid, tartaric acids, etc.

D.2 from Lactic Acid

The compounds obtained by the reductive amination reaction described above may be prepared as lactic acid salts for biological testing, according to the following procedure described above for the compound 007 with hydrochloric acid, by replacing hydrochloric acid by lactic acid.

Methyl-L-valinate 30-norspermidino-chenodeoxycholate 007 as lactic acid salt (007.3 lactic acid), namely compound S047, was prepared from compound 007 (007-a1, β/α: 96/4) according to this method and obtained as a pale yellow solid in quantitative yield.

D.3 from Citric Acid

The compounds obtained by the reductive amination reaction described above may be prepared as lactic acid salts for biological testing, according to the following procedure described above for the compound 007 with hydrochloric acid, by replacing hydrochloric acid by citric acid.

Methyl-L-valinate 30-norspermidino-chenodeoxycholate 007 as citric acid salt (007.3 citric acid), namely compound S048, was prepared from compound 007 (007-a1, β/α: 96/4) according to this method and obtained as a pale yellow solid in quantitative yield.

### E. Synthesis of a Diastereomeric Mixture

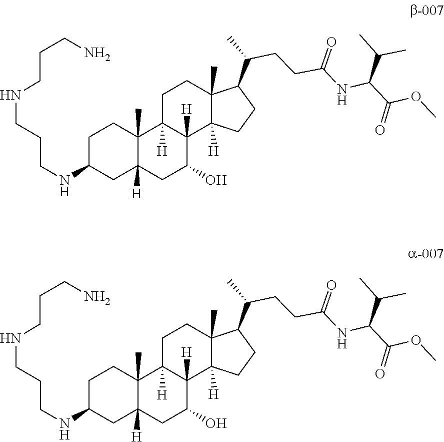

Diastereomeric mixture of methyl-L-valinate norspermidino-chenodeoxycholate (007-b1). In a 50 mL flask fitted with a magnetic stirring bar, 870 mg of methyl-L-valinate 3-oxo-chenodeoxycholate 15 (1.74 mmol) is dissolved in 30 mL of MeOH. Subsequently, 3 equivalents of norspermidine (0.73 mL, 5.21 mmol) and 4 equivalents of titanium tetra-isopropylate (2.06 mL, 6.96 mmol) are added. After 12 h stirring at 20° C., the flask is placed at 0° C. and 4 equivalents of sodium borohydride (50 mg, 1.36 mmol) are added while stirring. The reaction medium is left to stir at this temperature for 2 h. Then, 8.7 mL of water are added to neutralize the reaction. After 30 min of further stirring, the mixture was filtered over Celite and rinsed with MeOH, CH₂Cl₂ and EtOAc before being concentrated in vacuo. The crude product thus obtained is purified by chromatography on silica gel (eluent: MeOH then CH₂C₂/MeOH/NH₄·OH (7/3/1)). Methyl-L-valinate 30-norspermidino-chenodeoxycholate 007 is obtained, in the form of a yellow oil with a yield of 32% (mixture of two diastereomers (β/α) in a ratio (80/20) herein “007-b1” as a mixture). The corresponding hydrochloric salt S017 (β/α: 80/20) was prepared following the above-described method.

Following a similar method, Methyl-L-glycinate 30-spermino-chenodeoxycholate 004 was obtained, in the form of a yellow oil with a yield of 53% (mixture of two diastereomers (β/α) in a ratio (80/20) herein “004-b1” as a mixture). The corresponding hydrochloric salt S027 (β/α: 80/20) was prepared following the above-described method.

### Example 2: Intrinsic Anti-Bacterial Activities of the Compound

The purpose of this experiment was to test the anti-bacterial activity of the 61 compounds according to the invention (S001-S061).

### Materials and Methods

The antibacterial activity of compounds was measured using a standard microdilution assay based on the Clinical and Laboratory Standards Institute (CLSI) guidelines. This method was slightly modified. Indeed, assay volumes were increased to 200 μL to improve reproducibility. The chemical compounds to be tested were in the form of salts for biological testing.

Bacteria tested: Antibacterial activities of the compounds were tested on Staphylococcus aureus (ATCC25923), Enterococcus faecalis (ATCC29212), Escherichia coli (ATCC28922) and Pseudomonas aeruginosa (ATCC27853).

Preparation of the Preculture: Mueller Hinton Agar plates were inoculated with frozen biological strain in order to obtain separate colonies and the plates were incubated during 24 h at 35-37° C. 3 colonies of similar aspect were picked out, resuspended in 5 mL of fresh Mueller Hinton broth 1× (MHB) and incubated with shaking (160 rpm) at 35-37° C. overnight.

Preparation of the microplate for the Determination of the Minimum Inhibitory Concentration (MIC): 0.1 mL of overnight suspension were diluted in 10 ml of pre-warmed Mueller Hinton Broth 1× with shaking (160 rpm) at 35-37° C. until the culture reached an absorbance of 0.08-0.13 at 625 nm, corresponding to 10⁸ CFU/mL. 0.1 mL of bacteria in exponential phase were suspended in 9.9 mL of MHB (1:100 dilution). 0.1 mL of the 1:100 dilution were dispensed into the wells of microplates containing 0.1 ml of serial compounds dilutions to yield ca. 5×10⁵ CFU/mL. In parallel, the stocks solutions of chemical compounds were first diluted in appropriate solvent to a final concentration of 5 mg/mL. 8 μL were dispended into the first wells of the microplates containing 0.1 ml of sterile broth and a 0.5 cascade dilution was performed in MHB from 0.2 mg/mL. One control was made for each strain tested. MIC defined as the lowest concentration of compounds that inhibited growth, was determined after 20 hours incubation at 35-37° C. Tests were performed in triplicate.

Cytotoxicity: The test WST1 was used to measure the cytotoxic activity of the compounds of the invention. The test WST1 is a colorimetric test allowing to measure the viability and the degree of cell proliferation. It is based on cleavage of the colorless tetrazolium salts WST-1 (4—[3-(4-iodophenyl)-2-(4-nitrophenyl)-2H-5-tetrazolio]-1,3-benzene disulfonate) with mitochondrial dehydrogenases to the yellow-colored formazan derivative, which may be quantified by spectrophotometry at 420-480 nm. The WST1 test was performed on Chinese hamster ovary (CHO) cells. The CHO-K1 cells (ATCC, USA) were kept in culture in McCoy's 5A medium supplemented with 10% fetal calf serum, 2 mM of L-glutamine and a mixture of penicillin-streptomycin (100 U/ml: 10 μg/mL). The culture was incubated at 37° C. under an atmosphere enriched in CO₂ (5%), and subcultured every two days. The cells were transferred into 96-well plates (25,000 cells/mL) in whole McCoy's 5A medium, and maintained for 24 hours at 37° C. under a humid atmosphere enriched in CO₂ (5%). Increasing concentrations of tested compounds were added to the wells, each compound was tested in replicates and 8 growth controls containing the cells in the medium alone were included in each series of tests. After 24 hours at 37° C. (5% CO₂), the culture medium was removed, the cells were rinsed in phosphate buffer (PBS) and 50 μL of PBS containing 10% of reagent WST1 were added to each well. After 20 minutes of incubation at 37° C., the results were read by spectrophotometry at 450 nm. The results are expressed in dose-response relationships, modeled by nonlinear regression analysis using the TableCurve software. The 50% inhibitory concentration (ICso) represents the concentration of compound that is capable of reducing the cell viability by 50%.

Reading of the Results: After incubation, the filter was replaced with a transparent film, and an optical density reading was then taken in an iEMS plate spectrophotometer at 620 nm. Calculation of the minimum inhibitory concentration (MIC) was performed.

### Results

Results of the antibacterial activities of each compound are presented in Table 7 below.

As shown in Table 7, all tested compounds exhibit antibacterial activities against all tested bacteria. All compounds show the strongest antibacterial activity against Staphylococcus aureus.

TABLE 7
Anti-bacterial activity and cytotoxicity of the compounds.

| | Minimum inhibitory concentration | | | | |
|---|---|---|---|---|---|
| | (μM \| μg/mL) | | | | |
| | Gram-positive bacteria | | Gram-negative bacteria | | IC₅₀ |
| | S. | E. | E. | P. | (μM \| |
| Compound and | aureus | faecalis | coli | aeruginosa | μg/mL) |
| molar mass | ATCC25923 | ATCC29212 | ATCC28922 | ATCC27853 | CHO |
| S001 | 3.12* | 25 | 12.5 | 12.5* | 4.41 |
| 690.07 g/mol | 2* | 16 | 8 | 8* | 3.04 |
| S002 | 25 | 50 | 100 | 100 | 93.28 |
| 688.06 g/mol | 16 | 32 | 64 | 64 | 64.18 |
| S003 | 6.25* | 50 | 100 | 100 | 164.60 |
| 676.50 g/mol | 4* | 32 | 64 | 64 | 111.35 |
| S004 | 6.25* | 50* | 100 | >200* | 51.66 |
| 647.99 g/mol | 4* | 32* | 64 | >128* | 33.48 |
| S005 | 12.5 | 200 | >200 | >200* | >100 |
| 633.96 g/mol | 8 | 128 | >126 | >126* | >64 |
| S006 | 25 | >200 | >200 | >200 | 73.61 |
| 645.97 g/mol | 16 | >128 | >128 | >128 | 47.70 |
| S007 | 12.5 | 12.5 | 6.25 | 25 | 113.38 |
| 618.95 g/mol | 8 | 8 | 4 | 16 | 70.20 |
| S008 | 25* | 25 | 25 | 200 | 85.95 |
| 547.83 g/mol | 16 | 16 | 16 | 128 | 47.09 |
| S009 | 25 | 25 | 200 | >200 | 19.99 |
| 660.04 g/mol | 16 | 16 | 128 | >128 | 13.19 |
| S010 | 12.5 | 12.5 | 50 | 50 | 113.55 |
| 682.01 g/mol | 8 | 8 | 32 | 32 | 77.44 |
| S011 | 12.5* | 25 | 12.5* | 25* | 1.17 |
| 676.04 g/mol | 8* | 16 | 8 | 16* | 0.79 |
| S012 | 50 | 25 | 100 | 100 | 101.33 |
| 633.96 g/mol | 32 | 16 | 64 | 64 | 64.24 |
| S013 | 6.25* | 12.5* | 6.25* | 25* | 96.27* |
| 662.02 g/mol | 4* | 8* | 4* | 16* | 63.73 |
| S014 | 3.125* | 12.5* | 6.25* | 25* | 77.63 |
| 738.12 g/mol | 2* | 8* | 4* | 16* | 57.30 |
| S015 | 12.5* | 12.5* | 12.5* | 50* | >250 |
| 595.87 g/mol | 8* | 8* | 8* | 32* | >149 |
| S016 | 12.5* | 25 | 25* | 50 | 65.95 |
| 609.90 g/mol | 8* | 16 | 16* | 32 | 40.22 |
| S017 | 12.5* | 25* | 12.5* | 50* | 53.69 |
| 618.95 g/mol | | | | | |
| Dia: 80:20 | 8* | 16* | 8* | 32* | 33.23 |
| S018 | 50* | 100* | 200 | 200* | 10.86 |
| 561.85 g/mol | 32* | 64 | 128 | 128 | 6.10 |
| S019 | 6.25* | 25* | 12.5* | 12.5* | 97.09 |
| 618.95 g/mol | | | | | |
| Dia: 94:6 | 4* | 16* | 8* | 8* | 60.09 |
| S020 | 100 | >200 | 100* | >200* | 17.25 |
| 590.89 g/mol | 64 | >128 | 64 | >128 | 10.19 |
| S021 | 12.5 | 50 | 50 | 50 | — |
| 618.95 g/mol | 8 | 32 | 32 | 32 | — |
| Dia: 90:10 | | | | | |
| S022 | 12.5 | 25 | 12.5* | 12.5* | 17.07 |
| 724.09 g/mol | 8 | 16 | 8* | 8 | 12.36 |
| S023 | 50 | 100 | 100 | >200 | 48.50 |
| 603.93 g/mol | 32 | 64 | 64 | >120 | 29.29 |
| S024 | 25 | 25 | 25 | 100 | 83.71 |
| 638.94 g/mol | 16 | 16 | 16 | 64 | 53.49 |
| S025 | 25 | 25 | 25 | 100 | 3.70 |
| 736.10 g/mol | 16 | 16 | 16 | 64 | 2.72 |
| S026 | 25 | 50 | 25 | 200 | 11.38 |
| 632.98 g/mol | 16 | 32 | 16 | 126 | 7.20 |
| S027 | 6.25 | 25 | 12.5 | 50 | — |
| 647.99 g/mol | 4 | 16 | 8 | 32 | — |
| S028 | 12.5 | 12.5 | 6.25 | 50 | 54.60 |
| 666.99 g/mol | 8 | 8 | 4 | 32 | 36.42 |
| S029 | 12.5* | 12.5* | 50* | 50* | 47.77 |
| 710.06 g/mol | 8* | 8* | 32* | 32* | 33.92 |
| S030 | 100 | 200 | 50 | >200 | >500 |
| 623.92 g/mol | 64 | 124 | 32 | >124 | >311 |
| S031 | 6.25* | 25 | 12.5* | 25* | 84.55 |
| 681.02 g/mol | 4* | 16 | 8* | 16* | 57.58 |
| S032 | 50 | 12.5 | 50 | 200 | — |
| 708.09 g/mol | 32 | 8 | 32 | 128 | — |
| S034 | 50 | 6.25 | 50 | 100 | 265.63 |
| 633.96 g/mol | 32 | 4 | 32 | 64 | 168.40 |
| S035 | 12.5 | 12.5* | 12.5 | 12.5 | 45.23 |
| 676.04 g/mol | 8 | 8* | 8 | 8 | 30.58 |
| S036 | 50 | 6.25 | 6.25 | 12.5 | 50.51 |
| 704.10 g/mol | 32 | 4 | 4 | 8 | 35.56 |
| S037 | 6.25* | 6.25* | 6.25* | 25* | 126.63 |
| 724.09 g/mol | 4* | 4* | 4* | 16* | 91.69 |
| S038 | 3.13* | 6.25* | 6.25* | 12.5* | 44.96 |
| 662.02 g/mol | 2* | 4* | 4* | 8* | 29.76 |
| S039 | 6.25* | 12.5 | 6.25* | 6.25 | 60.02 |
| 754.11 g/mol | 4* | 8 | 4* | 4 | 45.26 |
| S040 | 25 | 25* | 50 | 50* | 155.16 |
| 682.99 g/mol | 16 | 16* | 32 | 32* | 105.97 |
| S041 | 12.5 | 12.5 | 12.5 | 12.5 | 9.80 |
| 676.04 g/mol | 8 | 8 | 8 | 8 | 6.63 |
| S042 | 6.25 | 25 | 12.5 | 25 | 51.37 |
| 726.06 g/mol | 4 | 16 | 8 | 16 | 37.30 |
| S043 | 25 | 25 | 50 | 200 | 107.46 |
| 575.77 g/mol | 16 | 16 | 32 | 128 | 61.88 |
| S044 | 6.25 | 6.25 | 6.25 | 12.5 | 52.39 |
| 688.10 g/mol | 4 | 4 | 4 | 8 | 36.89 |
| S045 | 3.13 | 6.25 | 6.25 | 25 | 12.60 |
| 616.93 g/mol | 2 | 4 | 4 | 16 | 7.77 |
| S046 | 12.5 | 6.25 | 12.5 | 12.5 | 9.53 |
| 660.00 g/mol | 8 | 4 | 8 | 8 | 6.29 |
| S047 | 6.25* | 12.5* | 12.5* | 12.5* | 93.09 |
| lactic acid salt | | | | | |
| 618.95 g/mol | 4* | 8* | 8* | 8* | 57.61 |
| S048 | 6.25* | 25* | 6.25* | 12.5* | 97.09 |
| citric acid salt | | | | | |
| 618.95 g/mol | 4* | 16* | 4* | 8* | 60.09 |
| S049 | 50 | 100 | 100 | 100 | 94.90 |
| 677.03 g/mol | 32 | 64 | 64 | 64 | 64.25 |
| S050 | 3.125 | 25 | 12.5 | 50 | 10.47 |
| 632.97 g/mol | 2 | 16 | 8 | 32 | 6.63 |
| S051 | 6.25 | 50 | 25 | 25 | 33.33 |
| 663.52 g/mol | 4 | 32 | 16 | 16 | 22.12 |
| S052 | 3.12* | 25 | 12.5* | 12.5* | 50 |
| 690.07 g/mol | 2* | 16 | 8 | 8 | 34 |
| S053 | 12.5 | 200 | 100 | >200 | >100 |
| 688.06 g/mol | 8 | 128 | 64 | >128 | >68 |
| S054 | 6.25 | 100 | 50 | 100 | >100 |
| 676.04 g/mol | 4 | 64 | 32 | 64 | >67 |
| S055 | 50 | 50 | 25 | 100 | — |
| 724.09 g/mol | 32 | 32 | 16 | 64 | — |
| S056 | 200 | 100 | 200 | >200 | — |
| 672.06 g/mol | 128 | 64 | 128 | >128 | — |
| S057 | 3.125 | 3.125 | 3.125 | 6.25 | 30.2 |
| 674.07 g/mol | 2 | 2 | 2 | 4 | 20.4 |
| S058 | 12.5* | 100 | 100 | 50* | 6.55 |
| 705.07 g/mol | 8* | 64 | 64 | 32* | 4.63 |
| S059 | 50* | >200 | 200 | >200* | 56.07 |
| M 704.6 g/mol | 32* | >128 | 128 | >128* | 39.48 |
| S060 | 40* | 160 | 160 | 160 | 186.16 |
| 692.04 g/mol | 32* | 128 | 128 | 128 | 128.83 |
| S061 | 6.25* | 100 | 100 | 100 | 6.85 |
| 770.60 g/mol | 4* | 64 | 64 | 64 | 5.17 |

*indicates experiments performed as triplicates.

### Example 3: Anti-Persister Activities of the Compounds

The purpose of this experiment was to test the anti-persister activity of 18 compounds according to the invention (S010, S013, S014, S017, S019, S039, S041, S042, S044, S046, S047, S048, S052, S060), as compared to the negative control ciprofloxacin.

### Materials and Methods

Anti-persister activity test: Escherichia coli (E. coli) persister cells were generated and isolated as described by MARQUES, C. N. H. et al. (Applied and Environmental Microbiology 2014, Vol. 80, No. 22, pp. 6976-6991). E. coli persister cells were compared to E. coli non-persister cells. 1 mL persister or non-persister bacteria culture was added in a 1.5 mL microtube with each of the compounds to be tested or ciprofloxacin as a negative control, and incubated at 37° C. with 160 rpm shaking for 4 hours. After 4 hours treatment with the compounds, the microtubes were centrifuged at 3,500g for 5 minutes and the pellets were resuspended in 1 mL of fresh drug free RM broth (10 g/L M9 salt, 2% casamino acids, 1 mM MgCl₂, 1% glycerol). Resuspended bacteria were then plated over LB agar plates and incubated at 37° C. Bacterial colonies were enumerated after 24, 48 and 96 hours to estimate the survival rate.

Cytotoxicity: The same WST1 test as described in Example 2 above was used.

### Results

Results of the survival rate obtained with each tested compound are presented in Table 8 below.

Compounds S046, S019, S039, S010, and S060 show low persister cell survival rates associated with higher non-persister cell survival rates. These results suggest that compounds S046, S019, S039, S010, and S060 have a very potent anti-persister activity. Additionally, S019, S039, S010, and S060 show good toxicity results.

Compounds S044, S052, S013, S047, S048 and S014 exhibit low persister and non-persister cell survival rates. This suggests that they have both anti-persister and anti-bacterial activities. In addition, they all show good toxicity results.

Compounds S042, S041 and S017 demonstrate a good activity on non-persister cells, with low non-persister cell survival rates, but their activity is not as good on persister cells, as shown by the higher persister cell survival rates. S042, and S017 have good toxicity results.

TABLE 8
Anti-persister activity and cytoxicity of the compounds

| | | Persister | Non- | | |
|---|---|---|---|---|---|
| | Concen- | cell | persister | Ratio | Toxicity |
| | tration | survival | cell survival | (Persisters/ | IC₅₀ |
| | used | rate 96 h | rate 96 h | Non- | CHO |
| Compound | (μM) | (%) | (%) | persisters) | (uM) |
| S046 | 125 | 0.0 | 0.3 | 0.0 | 9.53 |
| S019 | 250 | 0.0 | 4.0 | 0.0 | 97.09 |
| S039 | 62.5 | 0.0 | 2.7 | 0.0 | 60.02 |
| S010 | 250 | 0.8 | 4.2 | 0.2 | 113.55 |
| S060 | 250 | 2.0 | 3.8 | 0.5 | 186.16 |
| S044 | 62.5 | 0.0 | 0.0 | 0.0 | 52.35 |
| S052 | 62.5 | 0.0 | 0.0 | 0.0 | 50 |
| S013 | 250 | 0.0 | 0.0 | 0.0 | 96.27 |
| S047 | 125 | 0.0 | 0.0 | 0.0 | 93.09 |
| S048 | 125 | 0.0 | 0.0 | 0.0 | 97.09 |
| S014 | 125 | 0.0 | 0.0 | 0.0 | 77.63 |
| S042 | 125 | 0.1 | 0.0 | 312.2 | 51.37 |
| S041 | 125 | 0.1 | 0.0 | 4.7 | 9.8 |
| S017 | 250 | 0.6 | 0.0 | 39.8 | 53.69 |
| Cipro- | 200 | 5.6 | 0.0 | 1617.7 | — |
| floxacin | | | | | |

1-15. (canceled)

16. A compound of formula (I) 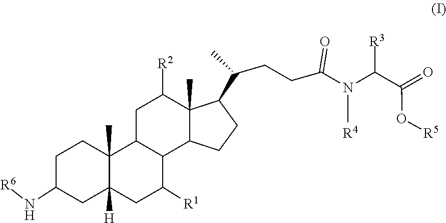
or a pharmaceutically acceptable salt and/or solvate thereof;
wherein:
R¹ represents H, OH, or SO₃H;
R² represents H, OH, or SO₃H;
R³ represents H, C₁-C₈alkyl, C₆-C₁₀ aryl, or C₆-C₁₀ aryl-C₁-C₈ alkyl;
wherein the alkyl group is optionally substituted by at least one OH, COOH, —C(O)NH₂, NH₂, —NH—C(═NH)—NH₂, imidazolyl, indolyl, SH, S—CH₃, or SeH;
wherein in the aryl or arylalkyl group, the aryl is optionally substituted by at least one OH;
R⁴ represents H, C₁-C₈alkyl or C₆-C₁₀ aryl;
or R³ and R⁴ form together with the nitrogen and carbon atoms to which they are attached
a 5-membered heterocycloalkyl;
R⁵ represents H, C₁-C₈alkyl, or C₆-C₁₀ aryl;
R⁶ represents —(CR⁷RB)ₘ—[X—(CR⁹R¹⁰)ₙ]ₚ—NR¹¹R¹²,
wherein
R⁷, R⁸, R⁹, and R¹⁰ represent, independently at each occurrence, and each independently, H, or C₁-C₈alkyl;
R¹¹ and R¹² represent, each independently, H or C₁-C₈ alkyl, or R¹¹ and R¹² form together with the nitrogen atom to which they are attached a 5- to 7-membered heterocyclyl optionally substituted by one to three R¹³;
wherein R¹³ represents ═O or ═S;
X represent, independently at each occurrence, —NR¹⁴— or a divalent 5- to
7-membered heterocycloalkyl comprising at least one nitrogen atom;
wherein R¹⁴ represents H, C₁-C₆ alkyl, or —(CH₂)_{q}—NH₂; wherein q represents an integer ranging from 1 to 5;
m is an integer ranging from 2 to 10;
n is an integer ranging from 1 to 5; and
p is an integer ranging from 0 to 4.

17. The compound according to claim 16, wherein R¹ and R² represent each independently H or OH.

18. The compound according to claim 16, wherein R³ represents H, C₁-C₈ alkyl, or phenyl-(CH₂)₂—, wherein the phenyl is optionally substituted by at least one OH; or wherein R³ and R⁴ form together with the nitrogen and carbon atoms to which they are attached a divalent pyrrolidine.

19. The compound according to claim 18, wherein R³ represents C₁-C₆ alkyl.

20. The compound according to claim 19, wherein R³ represents C₁-C₄ alkyl.

21. The compound according to claim 16, wherein R⁷ and R⁸ represent both H and/or R⁹ and R¹⁰ represent both H.

22. The compound according to claim 21, wherein R⁷, R⁸, R⁹, and R¹⁰ represent H.

23. The compound according to claim 16, wherein R¹¹ and R¹² represent both H.

24. The compound according to claim 16, wherein X represents —NR¹⁴— or a divalent piperazine, wherein R¹⁴ is as defined above.

25. The compound according to claim 24, wherein X represents —NR¹⁴—, wherein R¹⁴ is as defined above.

26. The compound according to claim 25, wherein X represents —NH-.

27. The compound according to claim 16, wherein m is 2 or 3; wherein n is 2, 3, or 4; and/or wherein p is 1 or 2.

28. The compound according to claim 16, wherein said compound is selected from the group consisting of:
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)valinate;
methyl((4R)-4-((3S,5S,7R,10S,13R,17R)-3-((3-(4-(3-aminopropyl)piperazin-1-yl)propyl)amino)-7-hydroxy-10,13-dimethylhexadecahydro-1H-cyclopenta[a]phenanthren-17-yl)pentanoyl)valinate;
methyl((4R)-4-((3S,5S,7R,10S,13R,17R)-3-((3-(bis(3-aminopropyl)amino)propyl)amino)-7-hydroxy-10,13-dimethylhexadecahydro-1H-cyclopenta[a]phenanthren-17-yl)pentanoyl)valinate;
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)glycinate;
methyl((4R)-4-((3S,5S,7R,10S,13R,17R)-3-((3-(bis(3-aminopropyl)amino)propyl)amino)-7-hydroxy-10,13-dimethylhexadecahydro-1H-cyclopenta[a]phenanthren-17-yl)pentanoyl)glycinate;
methyl((4R)-4-((3S,5S,7R,10S,13R,17R)-3-((3-(4-(3-aminopropyl)piperazin-1-yl)propyl)amino)-7-hydroxy-10,13-dimethylhexadecahydro-1H-cyclopenta[a]phenanthren-17-yl)pentanoyl)glycinate;
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((2-aminoethyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((10-aminodecyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((2-((2-((2-aminoethyl)amino)ethyl)amino)ethyl)amino)-7-hydroxy-10,13-dimethylhexadecahydro-1H-cyclopenta[a]phenanthren-17-yl)pentanoyl)-D-phenylalaninate;
methyl((4R)-4-((3S,5S,7R,10S,13R,17R)-3-((3-((3-((3-aminopropyl)amino)propyl)amino)propyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((2-((2-((2-aminoethyl)amino)ethyl)amino)ethyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-valinate;
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-phenylalaninate;
methyl((4R)-4-((3S,5S,7R,10S,13R,17R)-3-((2-aminoethyl)amino)-7-hydroxy-10,13-dimethylhexadecahydro-1H-cyclopenta[a]phenanthren-17-yl)pentanoyl)-L-phenylalaninate;
methyl((4R)-4-((3S,5S,7R,10S,13R,17R)-3-((3-aminopropyl)amino)-7-hydroxy-10,13-dimethylhexadecahydro-1H-cyclopenta[a]phenanthren-17-yl)pentanoyl)-L-phenylalaninate;
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((3-aminopropyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((2-((2-aminoethyl)amino)ethyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((3-((3-((3-aminopropyl)amino)propyl)amino)propyl)amino)-7-hydroxy-10,13-dimethylhexadecahydro-1H-cyclopenta[a]phenanthren-17-yl)pentanoyl)-D-phenylalaninate;
methyl((4R)-4-((3S,5S,7R,10S,13R,17R)-3-((6-aminohexyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((2-((2-aminoethyl)amino)ethyl)amino)-7-hydroxy-10,13-dimethylhexadecahydro-1H-cyclopenta[a]phenanthren-17-yl)pentanoyl)-L-phenylalaninate;
methyl((4R)-4-((3S,5S,7R,10S,13R,17R)-3-((3-(4-(3-aminopropyl)piperazin-1-yl)propyl)amino)-7-hydroxy-10,13-dimethylhexadecahydro-1H-cyclopenta[a]phenanthren-17-yl)pentanoyl)-L-phenylalaninate;
methyl((4R)-4-((3S,5S,7R,10S,13R,17R)-3-((3-((3-aminopropyl)(methyl)amino)propyl)amino)-7-hydroxy-10,13-dimethylhexadecahydro-1H-cyclopenta[a]phenanthren-17-yl)pentanoyl)-L-valinate;
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-phenylalaninate;
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-phenylalaninate;
methyl((4R)-4-((3S,5S,7R,10S,13R,17R)-3-((4-aminobutyl)amino)-7-hydroxy-10,13-dimethylhexadecahydro-1H-cyclopenta[a]phenanthren-17-yl)pentanoyl)-L-phenylalaninate;
methyl((4R)-4-((3S,5S,7R,10S,13R,17R)-3-((3-((3-aminopropyl)(methyl)amino)propyl)amino)-7-hydroxy-10,13-dimethylhexadecahydro-1H-cyclopenta[a]phenanthren-17-yl)pentanoyl)-L-phenylalaninate;
methyl((4R)-4-((3S,5S,7R,10S,13R,17R)-3-((10-aminodecyl)amino)-7-hydroxy-10,13-dimethylhexadecahydro-1H-cyclopenta[a]phenanthren-17-yl)pentanoyl)-L-phenylalaninate;
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-alaninate;
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-leucinate;
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-leucinate;
methyl((4R)-4-((3S,5S,7R,10S,13R,17R)-3-((3-(bis(3-aminopropyl)amino)propyl)amino)-7-hydroxy-10,13-dimethylhexadecahydro-1H-cyclopenta[a]phenanthren-17-yl)pentanoyl)-L-phenylalaninate;
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-alaninate;
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-tyrosinate;
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-L-tyrosinate;
methyl (2S,3S)-2-((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanamido)-3-methylpentanoate;
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-tyrosinate;
methyl((4R)-4-((3S,5S,7R,10S,13R,17R)-3-((4-aminobutyl)amino)-7-hydroxy-10,13-dimethylhexadecahydro-1H-cyclopenta[a]phenanthren-17-yl)pentanoyl)-L-valinate;
methyl (2S,3R)-2-((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanamido)-3-methylpentanoate;
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-prolinate;
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-D-prolinate;
methyl((4R)-4-((3S,5S,7R,10S,13R,17R)-3-((2-((2-((2-((2-aminoethyl)amino)ethyl)amino)ethyl)amino)ethyl)amino)-7-hydroxy-10,13-dimethylhexadecahydro-1H-cyclopenta[a]phenanthren-17-yl)pentanoyl)-L-valinate;
methyl (2S,3R)-2-((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanamido)-3-methylpentanoate;
methyl((4R)-4-((3S,5S,7R,10S,12S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)pentanoyl)glycinate;
methyl((4R)-4-((3S,5R,10S,12S,13R,17R)-3-((3-((4-((3-aminopropyl)amino)butyl)amino)propyl)amino)-12-hydroxy-10,13-dimethylhexadecahydro-1H-cyclopenta[a]phenanthren-17-yl)pentanoyl)-D-valinate;
methyl((4R)-4-((3S,5R,10S,12S,13R,17R)-3-((3-(4-(3-aminopropyl)piperazin-1-yl)propyl)amino)-12-hydroxy-10,13-dimethylhexadecahydro-1H-cyclopenta[a]phenanthren-17-yl)pentanoyl)-D-valinate;
methyl((4R)-4-((3S,5R,10S,12S,13R,17R)-3-((3-(bis(3-aminopropyl)amino)propyl)amino)-12-hydroxy-10,13-dimethylhexadecahydro-1H-cyclopenta[a]phenanthren-17-yl)pentanoyl)-D-valinate;
methyl((4R)-4-((3S,5R,10S,12S,13R,17R)-3-((3-(bis(3-aminopropyl)amino)propyl)amino)-12-hydroxy-10,13-dimethylhexadecahydro-1H-cyclopenta[a]phenanthren-17-yl)pentanoyl)-L-phenylalaninate;
methyl((4R)-4-((3S,5R,10S,13R,17R)-3-((3-(4-(3-aminopropyl)piperazin-1-yl)propyl)amino)-10,13-dimethylhexadecahydro-1H-cyclopenta[a]phenanthren-17-yl)pentanoyl)-L-valinate;
methyl((4R)-4-((3S,5R,10S,13R,17R)-3-((3-((4-((3-aminopropyl)amino)butyl)amino)propyl)amino)-10,13-dimethylhexadecahydro-1H-cyclopenta[a]phenanthren-17-yl)pentanoyl)-L-valinate;
methyl((4R)-4-((3S,5S,7R,10S,12S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)pentanoyl)-D-valinate;
methyl((4R)-4-((3S,5S,7R,10S,12S,13R,17R)-3-((3-(4-(3-aminopropyl)piperazin-1-yl)propyl)amino)-7,12-dihydroxy-10,13-dimethylhexadecahydro-1H-cyclopenta[a]phenanthren-17-yl)pentanoyl)-D-valinate;
methyl((4R)-4-((3S,5S,7R,10S,12S,13R,17R)-3-((3-(bis(3-aminopropyl)amino)propyl)amino)-7,12-dihydroxy-10,13-dimethylhexadecahydro-1H-cyclopenta[a]phenanthren-17-yl)pentanoyl)-D-valinate; and
methyl((4R)-4-((3S,5S,7R,10S,12S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)pentanoyl)-D-tyrosinate;
and pharmaceutically acceptable salts and/or solvates thereof.

29. The compound according to claim 28, wherein said compound is selected from the group consisting of:
methyl((4R)-4-((3S,5S,7R,10S,13R,17R)-3-((2-((2-((2-aminoethyl)amino)ethyl)amino)ethyl)amino)-7-hydroxy-10,13-dimethylhexadecahydro-1H-cyclopenta[a]phenanthren-17-yl)pentanoyl)-D-phenylalaninate;
methyl((4R)-4-((3S,5S,7R,10S,13R,17R)-3-((3-((3-aminopropyl)amino)propyl)amino)-7-hydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-tyrosinate;
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-D-prolinate;
methyl((4R)-4-((3S,5S,7R,10S,12S,13R,17R)-3-((3-(bis(3-aminopropyl)amino)propyl)amino)-7,12-dihydroxy-10,13-dimethylhexadecahydro-1H-cyclopenta[a]phenanthren-17-yl)pentanoyl)-D-valinate;
methyl((4R)-4-((3S,5S,7R,10S,13R,17R)-3-((3-((2-((3-aminopropyl)amino)ethyl)amino)propyl)amino)-7-hydroxy-10,13-dimethylhexadecahydro-1H-cyclopenta[a]phenanthren-17-yl)pentanoyl)-L-valinate;
methyl((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanoyl)-L-phenylalaninate;
methyl (2S,3R)-2-((4R)-4-((3S,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)pentanamido)-3-methylpentanoate; and
methyl((4R)-4-((3S,5R,10S,12S,13R,17R)-3-((3-((4-((3-aminopropyl)amino)butyl)amino)propyl)amino)-12-hydroxy-10,13-dimethylhexadecahydro-1H-cyclopenta[a]phenanthren-17-yl)pentanoyl)-D-valinate;
and pharmaceutically acceptable salts and/or solvates thereof.

30. A process for manufacturing the compound as defined in claim 16, comprising:
(a) a step of reacting the carboxylic acid function in position 20 of a bile acid of formula (A) 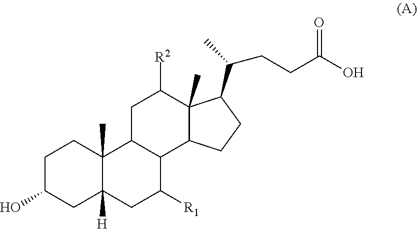
with the secondary amine function of an amino acid of formula 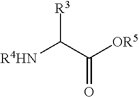
thereby obtaining an amide;
(b) a step of oxidizing the hydroxyl (OH) in position 3 of the bile acid intermediate obtained in step (a), thereby obtaining a ketone;
(c) a step of reacting the ketone of the bile acid intermediate obtained in step (b) with a primary amine of formula R⁶NH₂, thereby obtaining an imine; and
(d) a step of reduction of the imine obtained in step (c), thereby obtaining the compound of formula (I) or the pharmaceutically acceptable salt and/or solvate thereof.

31. A method for treating an infectious disease in a subject in need thereof, comprising administering to the subject the compound as defined in claim 16.

32. The method according to claim 31, wherein the infectious disease is a bacterial disease, a viral disease, a fungal disease, or a parasitic disease.

33. The method according to claim 31, wherein the infectious disease is:
a bacterial or fungal disease selected from cystic fibrosis, urinary tract infection, and chronic otitis; and/or
an infectious disease caused by Gram-positive bacteria selected from Staphylococcus bacteria, Enterococcus bacteria, and Mycobacterium bacteria.

34. The method according to claim 31, wherein the compound is used as an anti-persisters agent in the treatment of the infectious disease.

35. The method according to claim 34, wherein the infectious disease is a bacterial disease or a fungal disease.
